(19) United States
(12) Patent Application Publication
(10) Pub. No.: US 2026/0356963 A1
(43) Pub. Date: Nov. 12, 2026

(54) UL141 VARIANTS, COMPOSITIONS THEREOF AND METHODS OF USING THE SAME FOR CANCER IMMUNOTHERAPY

(71) Applicant: THE REGENTS OF THE UNIVERSITY OF CALIFORNIA, Oakland, CA (US)

(72) Inventors: Jogender TUSHIR-SINGH, Oakland, CA (US); Sanchita BHATNAGAR, Oakland, CA (US)

(21) Appl. No.: 19/108,003

(22) PCT Filed: Aug. 31, 2023
(86) PCT No.: PCT/US2023/031776

§ 371 Date: Feb. 28, 2025

Related U.S. Application Data
(60) Provisional application No. 63/374,210, filed on Aug. 31, 2022.
(60) Provisional application No. 63/383,769, filed on Nov. 15, 2022.

(51) Int. Cl. C07K 16/28; A61P 35/00; C07K 14/005; C07K 14/705

(52) U.S. Cl. CPC C07K 16/2875; A61P 35/00; C07K 14/005; C07K 14/70517; C07K 14/70521; C07K 14/70578; C07K 16/28; C07K 2317/31; C07K 2319/00

(57) ABSTRACT

The present disclosure provides UL141 variants with one or more mutations, pharmaceutical compositions, diagnostic compositions, and kits containing the variants, nucleic acids and expression vectors encoding the variants, cells comprising the same, and methods of using the variants, nucleic acids, expression vectors, and cells for therapeutic and diagnostic purposes.

### CROSS-REFERENCE TO RELATED APPLICATIONS

This application claims the benefit of U.S. Provisional Application No. 63/374,210, filed Aug. 31, 2022, and U.S. Provisional Application No. 63/383,769, filed Nov. 15, 2022, each of which is incorporated herein by reference in their entirety.

### STATEMENT OF GOVERNMENT INTEREST

This invention was made with government support under Grant No. R01CA233752 awarded by National Institutes of Health. The government has certain rights in the invention.

### REFERENCE TO ELECTRONIC SEQUENCE LISTING

This application contains a Sequence Listing which has been submitted electronically in .XML format and is hereby incorporated by reference in its entirety. Said .XML copy, created on Aug. 28, 2023, is named “043882.00071.xml” and is 105,845 bytes in size. The sequence listing contained in this .XML file is part of the specification and is hereby incorporated by reference herein in its entirety.

### FIELD OF THE INVENTION

The present disclosure generally relates to UL141 variant polypeptides with one or more mutations, which illustrate improved effects (e.g., increased death receptor 5 (DR5) activation and/or increased TIGIT signaling inhibition via saturating CD155) as potential cancer therapeutics capable of co-targeting cell death and immune checkpoint pathway by a single agent. Also provided are pharmaceutical compositions, diagnostic compositions and kits containing the variant polypeptides disclosed herein, nucleic acids and expression vectors encoding the variant polypeptides disclosed herein, cells comprising the same, and methods of using the variant polypeptides, nucleic acids, expression vectors, and cells for cancer therapeutic, and diagnostic purposes.

### BACKGROUND

The overall cancer death rates have been down ~20% in the past 25 years; however, mortality rates in ovarian, triple-negative breast, colon, and other solid cancers have remained relatively unchanged in the last two decades. For instance, in the case of ovarian cancer, the average clinical response of cancer immunotherapy and chimeric antigen receptor T-cells (CAR-T) strategies is only 0-10% compared to other tumors, especially the liquid tumors such as leukemia (Nixon et al., 2018, Curr Oncol. 25(5):e373-e84). Similar is the case of triple negative breast cancer (TNBC), where anti-PD-L1 therapies only improve survival by about 3 months. Similarly, other solid tumors such as glioblastoma (GBM) and gastric cancer have limited clinical success from immunotherapy. Importantly, most solid tumors represent an extreme immune desert microenvironment compared to liquid tumors. This limits the infiltration of immune effector cells in the hypoxic and anergic solid tumors, forming a bottleneck to the success of cancer immunotherapy in many solid tumors (Sasaki et al., 2015, Invest New Drugs. 33(2):332-40; Lin et al., 2013, Cancer Biol Ther. 14(11):1032-8; Gras Navarro et al., 2015, Front Immunol. 2015; 6:202; Leone et al., 2015, Cancer Cell. 27(4):435-6; Song et al., 2013, Cancer Lett. 339(1):70-81; Wek et al., 2010, EMBO J. 29(12):1946-7). As a result, immunotherapy and CAR-T therapy remains challenging for most solid tumors.

Cancer immunotherapy uses antibody-based approaches to activate immune cells against cancer cells and has proven effective in blood cancers and melanomas. However, most solid tumors tested for immunotherapy have been significantly discouraged compared to liquid tumors in clinical trials. This has been attributed to 1) the limited infiltration of activated immune effector cells into the solid tumor bed, and 2) most immunotherapy agents targeting one particular mechanism to eliminate cancer cells. The compositions and methods described herein focus on overcoming the latter by co-targeting the multiple cancer cell elimination mechanisms simultaneously to enhance the power of immunotherapy for solid tumor therapy.

As an alternative to immunotherapy, epithelial cell-enriched death receptor-5 (DR5/TRAIL-R2) agonist antibodies, which generate tumor debulking and shrinkage by activating extrinsic apoptosis independent of immune effector cells, provide a potential therapeutic strategy for solid tumors such as OvCa, TNBC, gastric, colon and GBM. DR5 is a key epithelial enriched TNF superfamily member. Due to its extrinsic tumor breaking cytotoxic properties, it offers a potential to overcome the immune infiltration bottleneck in solid tumors. However, clinically tested DR5 agonist antibodies have been largely disappointing in improving the survival beyond phase-II trials of various cancers (Wajant, 2019, Cancers (Basel) 11(7)). Recently, a patch of positively charged residues (residues 101-104 having the sequence RKCR) was identified to have a negative regulatory function in the CRD3 domain of DR5 (Shivange et al., 2021, Cell Rep. 37(5):109953).

New candidates for treating solid tumors are needed.

### SUMMARY

Provided herein are UL141 variants comprising one or more mutations described herein. In certain embodiments, the UL141 variants are capable of specifically engaging the patch of positively charged residues (PPCR) of the CRD3 domain of DR5 (i.e., residues 101-104 having the sequence RKCR) to activate apoptotic signaling. In certain embodiments, the variants specifically bind the PPCR of DR5. In certain embodiments, the variants specifically bind residues 101-104 of DR5. In certain embodiments, the variants specifically bind the sequence RKCR of DR5. In certain embodiments, provided herein are compounds or macromolecules that specifically bind the PPCR of DR5 and inhibit CD155-TIGIT.

In certain embodiments, the UL141 variants provide DR5 activation. In certain embodiments, the UL141 variants enhance DR5 clustering. In certain embodiments, the UL141 variants enhance DR5 signaling. In certain embodiments, the UL141 variants enhance apoptotic signaling. In certain embodiments, the UL141 variants provide increased CD155 saturating binding. In certain embodiments, the UL141 variants provide CD155 inhibition. In certain embodiments, the UL141 variants provide CD155-TIGIT inhibition. In certain embodiments, the UL141 variants provide TIGIT signaling inhibition, for instance, comparable to clinical anti-TIGIT antibodies such as Tiragolumab. In certain embodiments, the UL141 variants provide increased DR5 activation and TIGIT inhibition. In certain embodiments, the UL141 variants provide T-cell activation. In certain embodiments, the UL141 variants provide simultaneous tumor specific DR5 activation and T-cell activation. In certain embodiments, the UL141 variants inhibit CD155-TIGIT pairing and T-cell inhibitory signaling. In certain embodiments, the UL141 variants inhibit TIGIT clustering by CD155. In certain embodiments, the UL141 variants do not interfere with CD155-CD226 binding. In certain embodiments, the UL141 variants are in in a bispecific format, for instance, to target a tumor enriched antigen, for example FOLR1-DR5, useful for cancer, for example ovarian cancer. In certain embodiments, the UL141 variants are useful combination of immune checkpoint receptor inhibitors such as inhibitors of PD-L1, TIGIT, PD-1, CTLA, TIM3, LAG3, or the like.

In one aspect, provided herein are UL141 variants. In certain embodiments, the UL141 variants comprise at least one mutation selected from the group consisting of: L93A, D96N, V98D, K99L, Y113F, F127Y, R146H, N147T, S149P, H150D, R240E, Y241P, R242E, and combinations thereof. In certain embodiments, the mutations are relative to SEQ ID NO:1, provided herein. In certain embodiments, the UL141 variants have at least 70%, 80%, 85%, 90%, or 95% sequence identity to SEQ ID NO:1. In certain embodiments, the UL141 variants have increased activation of DR5. In certain embodiments, the UL141 variants have increased inhibition of CD155. In certain embodiments, the UL141 variants have increased activation of DR5 and increased inhibition of CD155.

In another aspect, provided herein are fusions comprising the UL141 variants described herein. In another aspect, provided herein are polynucleotides encoding the UL141 variants or fusions described herein. In a further aspect, provided herein are expression vectors comprising the polynucleotides. In a further aspect, provided herein are cells comprising the polynucleotides or expression vectors. In some embodiments, the cells are selected from a bacterial cells, fungal cell and mammalian cells. In some embodiments, the cells are selected from E. coli cells, Saccharomyces cerevisiae cells, and CHO cells.

In another aspect, provided herein are methods of treating, preventing or diagnosing a disease or condition in a subject in need thereof, wherein the method includes administering to the subject an effective amount of the UL141 variant of any of the foregoing embodiments, or a composition or a pharmaceutical composition containing the same. In some embodiments, the disease or condition is selected from a cancer, for instance a solid tumor. In some embodiments, the effective amount is a therapeutically effective amount.

Embodiments disclosed herein are also directed to the use of the UL141 variant of any of the foregoing embodiments for treating, preventing or diagnosing a disease or condition in a subject in need thereof. Embodiments disclosed herein are also directed the UL141 variant of any of the foregoing embodiments for use in treating, preventing or diagnosing a disease or condition in a subject in need thereof. Embodiments disclosed herein are also directed the UL141 variant of any of the foregoing embodiments for use the manufacture of a medicament for treating, preventing or diagnosing a disease or condition in a subject in need thereof. In some embodiments, the disease or condition is selected from a cancer, for instance a solid tumor.

These and other embodiments along with many of its features are described in more detail in conjunction with the text below and attached figures.

### BRIEF DESCRIPTION OF THE FIGURES

FIG. 1 (A,B) provides HCMV UL141 protein-mediated destabilization and blockade of DR5 oligomerization in infected OvCa cells.

FIG. 2 (left, right) provides a working model of hCMV bug derived drug: recombinant UL141 variant IgG1 Fc.

FIG. 3 (A-D) provides PPCR epitope remains unengaged by HCMV UL141 protein binding to DR5.

FIG. 4 (A-C) provides higher DR5 profile in OvCa and inverse relation of HCMV infection in OvCa.

FIG. 5 (A-C) provides engineered UL141 variant protein is a multi-prong effector molecule to target OvCa tumors.

FIG. 6 (A-C) provides HCMV UL141 (but not UL144) interferes with apoptotic activity of clinical DR5 antibodies.

FIG. 7 (A-C) provides unlike DR5 activating ligand Apo2L and antibody Apomab, DR5 destabilizing hCMV UL141 contain positive charge residues at DR5 PPCR interface

FIG. 8 (A-D) provides reverse engineering of hCMV derived UL141 for an extrinsic (outside-in) DR5 apoptotic activator.

FIG. 9 (A-F) provides lead UL141 variant (UL141^{D11}) is highly cytotoxic to Ovarian and other DR5⁺ tumors.

FIG. 10 (A-D) provides improving UL141^{D11} yield in CHO expression system.

FIG. 11 (A-D) provides UL141 mirrors TIGIT binding interface with CD155.

FIG. 12 (A-D) provides UL141 variant (UL141^{D11(2)}) binds CD155 with high affinity.

FIG. 13 (A-C) provides UL141^{D14(2)} inhibits CD155-TIGIT pairing and T-cell inhibitory signaling. (A)

FIG. 14 (A-D) provides UL141^{D14(2)} inhibits CD155-TIGIT clustering, pairing and T-cell inhibitory signaling.

FIG. 15 provides UL141^{D11} CAR-T or CAR-NK constructs.

FIG. 16 provides fluorescent micrographs to show DR5 and UL141 localization.

FIGS. 17 (A-B) provide a detailed schematic of mixing experiments (A) and results for the precipitates from the pull down and leftover supernatants (B).

FIGS. 18 (A-D) provide binding kinetics for immobilized CD155 against UL141 variants (A), immunoprecipitation results of CD155 (OVCAR-3 cells) using UL141^{D11(2)} IgG1 Fc (B), a schematic showing anti-FOLR1 and anti-DR5 lextumumab and UL141^{D11(2)}-Lexa bispecifics, and a cell killing assay of UL141^{D11(2)} alone, BaCa and UL141^{D11(2)}-Lexa in OVCAR-3 cells.

FIGS. 19 (A-D) provide a schematic for testing CD155-TIGIT pairing (A), immunoblots for the assays (B), a schematic for assaying activation markers (C) and the results of the activation marker assay (D).

### DETAILED DESCRIPTION OF THE EMBODIMENTS

Provided herein are UL141 variants and compositions comprising the same, wherein the UL141 variants comprise at least one amino acid substitution relative to a wild type UL141. As disclosed herein, the at least one amino acid substitution at a specific site can improve the characteristics of the UL141 variant relative to a wild type (i.e., parent) UL141. For example, amino acid substitutions as disclosed herein can lead to reduced receptor binding and/or reduced toxicity, relative to a wild type UL141. This can lead to advantages with respect to the use of UL141 variant in therapy or diagnosis.

### DEFINITIONS

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4^{th} ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value±10%, ±5%, or ±1%. In certain embodiments, the term “about” indicates the designated value±one standard deviation of that value.

The term “combinations thereof” includes every possible combination of elements to which the term refers to.

“UL141” refers to cytomegalovirus protein that blocks surface expression of DR5 in cells. Sequences include GenBank AY600468 (DNA) and AAT68307 (protein). A representative UL141 sequence is provided by SEQ ID NO:66:

```
(SEQ ID NO: 66)
    1 MCRRESLRTL PWLFWVLLSC PRLLEYSSSS
        FPFATADIAE KMWAENYETT SPAPVLVAEG
  61 EQVTIPCTVM THSWPMVSIR ARFCRSHDGS
        DELILDAVKG HRLMNGLQYR LPYATWNFSQ
121 LHLGQIFSLT FNVSTDTAGM YECVLRNYSH
        GLIMQRFVIL TQLETLSRPD EPCCTPALGR
181 YSLGDQIWSP TPWRLRNHDC GMYRGFORNY
        FYIGRADAED CWKPACPDEE PDRCWTVIQR
241 YRLPGDCYRS QPHPPKFLPV TPAPPADIDT
        GMSPWATRGI AAFLGFWSIF TVCFLCYLCY
301 LQCCGRWCPT PGRGRRGGEG YRCLPTYDSY
        PGVKKMKR.
```

A representative mature form UL141 sequence (residues 30-278) is provided by SEQ ID NO:1. For clarity, the first residue of SEQ ID NO:1 is numbered 30, and the residues of SEQ ID NO:1 are numbered through 278 herein.

(SEQ ID NO: 1)
SFPFATADIA EKMWAENYET TSPAPVLVAE
GEQVTIPCTV MTHSWPMVSI RARFCRSHDG
SDELILDAVK GHRLMNGLQY RLPYATWNFS
QLHLGQIFSL TENVSTDTAG MYECVLRNYS
HGLIMQRFVI LTQLETLSRP DEPCCTPALG
RYSLGDQIWS PTPWRLRNHD CGMYRGFORN
YFYIGRADAE DCWKPACPDE EPDRCWTVIQ
RYRLPGDCYR SQPHPPKFLP VTPAPPADID
TGMSPWATR.

“Death receptor 5” or “DR5” refers to a cell surface receptor of the TNF-receptor superfamily that binds TRAIL and mediates apoptosis. Sequences include NM_003842 and NM_147187 (mRNA) and NP_003833 and NP_671716 (protein).

“Cluster of differentiation 155” or “CD155” refers to a transmembrane protein in the immunoglobulin superfamily that functions in establishing adherens junctions between epithelial cells. It is also known as poliovirus receptor or PVR. Sequences include NM_001135768, NM_001135769, NM_001135770, and NM_006505 (mRNA) and NP_001129240, NP_001129241, NP_001129242, and NP_006496A (protein).

The term “wild-type” or “parent” refers to a naturally occurring gene or protein. These include a naturally occurring UL141 gene or protein.

The term “variant” refers to a gene or protein encoding or having one or more amino acid additions, deletions, or substitutions compared to a parent gene or protein.

An “isolated UL141 variant” is one that has been separated and/or recovered from a component of its natural environment. Components of the natural environment may include enzymes, hormones, and other proteinaceous or nonproteinaceous materials. In some embodiments, an isolated UL141 variant is purified to a degree sufficient to obtain at least 15 residues of N-terminal or internal amino acid sequence, for example by use of a spinning cup sequenator. In some embodiments, an isolated UL141 variant is purified to homogeneity by gel electrophoresis (e.g., SDS-PAGE) under reducing or nonreducing conditions, with detection by Coomassie blue or silver stain. In some aspects, an isolated UL141 variant is prepared by at least one purification step.

The term “substantially pure” with respect to a composition comprising a UL141 variant refers to a composition that includes at least 80%, 85%, 90% or 95% by weight or, in certain embodiments, 95%, 98%, 99% or 100% by weight, e.g., dry weight, of the UL141 variant relative to the remaining portion of the composition. The weight percentage can be relative to the total weight of protein in the composition or relative to the total weight of UL141 variant in the composition. Purity can be determined by techniques apparent to those of skill in the art, for instance SDS-PAGE.

In some embodiments, an isolated UL141 variant is purified to at least 80%, 85%, 90%, 95%, or 99% by weight. In some embodiments, an isolated UL141 variant is purified to at least 80%, 85%, 90%, 95%, or 99% by volume. In some embodiments, an isolated UL141 variant is provided as a solution comprising at least 85%, 90%, 95%, 98%, 99% to 100% by weight. In some embodiments, an isolated UL141 variant is provided as a solution comprising at least 85%, 90%, 95%, 98%, 99% to 100% by volume.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution an amino acid with a chemically or functionally similar amino acid. Conservative substitution tables providing similar amino acids are well known in the art. Polypeptide sequences having such substitutions are known as “conservatively modified variants.” By way of example, the groups of amino acids provided in Tables 1-3 are, in some embodiments, considered conservative substitutions for one another.

TABLE 1
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 2
Additional selected groups of amino acids that
are considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 3
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, NY. A UL141 variant generated by making one or more conservative substitutions of amino acid residues in a parent UL141 is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “therapeutically effective amount” or “effective amount” refers to an amount of a UL141 variant or composition that when administered to a subject is effective to treat a disease or disorder.

As used herein, the term “subject” means a mammalian subject. Exemplary subjects include, but are not limited to humans, monkeys, dogs, cats, mice, rats, cows, horses, camels, avians, goats, and sheep. In certain embodiments, the subject is a human. In some embodiments, the subject has a cancer, an inflammatory disease or condition, or an autoimmune disease or condition, that can be treated or diagnosed with a UL141 variant provided herein. In some embodiments, the subject is a human that has or is suspected to have cancer, an inflammatory disease or condition, or an autoimmune disease or condition.

### Introduction

While not intending to be bound by any particular theory of operation, the present disclosure is based in part on a DR5 epitope near its patch of positively charged residues in the third cysteine-rich domain (Shivange et al., 2021, Cell Rep. 2021; 37(5):109953). The DR5-binding negative regulatory interface of UL141 was modified by directed evolution and reverse-engineering to generate DR5 activating (rather than inhibitory) variants of UL141.

The lead variant named UL141^{D11} when tested against OvCa cells and tumors, had efficacy comparable to preclinical and clinical DR5 antibodies. However, unlike clinical DR5 antibodies, the affinity matured variant of UL141^{D11(2)} is also a high-affinity competitor of immune checkpoint ligand named poliovirus receptor (PVR or CD155) on tumor cells. Thus, in addition to activating cytotoxic DR5 signaling, UL141^{D11(2)} also interferes with T-cell inhibiting CD155-TIGIT (T-cell receptor with Ig and ITIM domain) interactions. Unlike very large-size limitedly tumor penetrable dual specificity antibodies, a small size dual functioning single-agent UL141^{D11(2)} with high capability to penetrate ovarian tumors offers a vast potential to be as effective solid tumor drugs like no other.

### UL141 Variants

Provided herein are UL141 variants that comprise at least one amino acid mutation compared to a wild type UL141 polypeptide. In some embodiments, the UL141 variants comprise at least two amino acid mutation. In some embodiments, the UL141 variants comprise at least three, four, five, six, or more amino acid mutations. In certain embodiments, the mutations are insertions, deletions, or substitutions. In certain embodiments, the mutations are substitutions. In certain embodiments, the UL141 variants are affinity matured.

The at least one amino acid mutation can be made by standard techniques. In certain embodiments, the mutation is made by one or more mutations in the genetic sequence encoding the UL141 variants.

In some embodiments, a UL141 variant comprises an amino acid substitution in at least one amino acid position selected from the group consisting of: L93A, D96N, V98D, K99L, Y113F, F127Y, R146H, N147T, S149P, H150D, R240E, Y241P, R242E, and combinations thereof. In some embodiments, a UL141 variant comprises two of the amino acid substitutions. In some embodiments, a UL141 variant comprises three of the amino acid substitutions. In some embodiments, a UL141 variant comprises four of the amino acid substitutions. In some embodiments, a UL141 variant comprises five of the amino acid substitutions. In some embodiments, a UL141 variant comprises six of the amino acid substitutions. In some embodiments, a UL141 variant comprises seven of the amino acid substitutions. In some embodiments, a UL141 variant comprises eight of the amino acid substitutions. In some embodiments, a UL141 variant comprises nine of the amino acid substitutions. In some embodiments, a UL141 variant comprises ten of the amino acid substitutions. In some embodiments, a UL141 variant comprises eleven of the amino acid substitutions. In some embodiments, a UL141 variant comprises more than eleven of the amino acid substitutions. In some embodiments, the amino acid substitution is with reference to SEQ ID NO:1, where the first residue of SEQ ID NO:1 is numbered 30, and the remaining residues are numbered through 278, corresponding to the sequence of wild-type UL141.

In some embodiments, a UL141 variant comprises an amino acid substitution in at least one amino acid position selected from the group consisting of: L93A, D96N, V98D, K99L, Y113F, R146H, N147T, S149P, H150D, R240E, R242E, and combinations thereof. In some embodiments, a UL141 variant comprises two of the amino acid substitutions. In some embodiments, a UL141 variant comprises three of the amino acid substitutions. In some embodiments, a UL141 variant comprises four of the amino acid substitutions. In some embodiments, a UL141 variant comprises five of the amino acid substitutions. In some embodiments, a UL141 variant comprises six of the amino acid substitutions. In some embodiments, a UL141 variant comprises seven of the amino acid substitutions. In some embodiments, a UL141 variant comprises eight of the amino acid substitutions. In some embodiments, a UL141 variant comprises nine of the amino acid substitutions. In some embodiments, a UL141 variant comprises ten of the amino acid substitutions. In some embodiments, a UL141 variant comprises eleven of the amino acid substitutions. In some embodiments, the amino acid substitution is with reference to SEQ ID NO: 1.

In some embodiments, a UL141 variant comprises an amino acid substitution in at least one amino acid position selected from the group consisting of: D96N, V98D, K99L, F127Y, S149P, R240E, Y241P, R242E, and combinations thereof. In some embodiments, a UL141 variant comprises two of the amino acid substitutions. In some embodiments, a UL141 variant comprises three of the amino acid substitutions. In some embodiments, a UL141 variant comprises four of the amino acid substitutions. In some embodiments, a UL141 variant comprises five of the amino acid substitutions. In some embodiments, a UL141 variant comprises six of the amino acid substitutions. In some embodiments, a UL141 variant comprises seven of the amino acid substitutions. In some embodiments, a UL141 variant comprises eight of the amino acid substitutions. In some embodiments, the amino acid substitution is with reference to SEQ ID NO:1.

In certain embodiments, the UL141 variant comprises the mutations R240E and R242E. In certain embodiments, the UL141 variant comprises the mutations R240E, Y241P, and R242E. In certain embodiments, the UL141 variant comprises at least one mutation selected from the group consisting of: L93A, D96N, V98D, K99L, Y113F, R146H, N147T, S149P, H150D, R240E, R242E, and combinations thereof. In certain embodiments, the UL141 variant comprises each of the following mutations: L93A, D96N, V98D, K99L, Y113F, R146H, N147T, S149P, H150D, R240E, and R242E. In certain embodiments, the UL141 variant comprises at least one mutation selected from the group consisting of: D96N, V98D, K99L, F127Y, S149P, R240E, Y241P, R242E, and combinations thereof. In certain embodiments, the UL141 variant comprises each of the following mutations: D96N, V98D, K99L, F127Y, S149P, R240E, Y241P, R242E.

In certain embodiments, the UL141 variant further comprises a mutation selected from the group consisting of Q162L, T165del, L166del, Q207S, I238D, Q239E, R240D, R240G, R240S, Y241D, Y241S, R242D, R242Y, R242P, 242-insD-243, 242-insE-243, R251E, and combinations thereof. In certain embodiments, the UL141 variant further comprises a mutation selected from the group consisting of 30-161del, 173-279del, 183-279del, 193-279del, 203-279del, 213-279del, 223-279del, 233-279del, 218-223del, 271-279del, 163-279del, 208-279del, 217-279del, and 228-279del.

In some embodiments, the UL141 variant has at least 70% sequence identity to SEQ ID NO:1. In some embodiments, the UL141 variant has at least 75% sequence identity to SEQ ID NO:1. In some embodiments, the UL141 variant has at least 80% sequence identity to SEQ ID NO:1. In some embodiments, the UL141 variant has at least 85% sequence identity to SEQ ID NO:1. In some embodiments, the UL141 variant has at least 90% sequence identity to SEQ ID NO:1. In some embodiments, the UL141 variant has at least 95% sequence identity to SEQ ID NO:1. In some embodiments, the UL141 variant has at least 96% sequence identity to SEQ ID NO:1. In some embodiments, the UL141 variant has at least 97% sequence identity to SEQ ID NO:1. In some embodiments, the UL141 variant has at least 98% sequence identity to SEQ ID NO: 1. In some embodiments, the UL141 variant has at least 99% sequence identity to SEQ ID NO: 1.

In particular embodiments, UL141 variants according to SEQ ID NOS:2-33 are provided.

Also within the scope are post-translationally modified variants of the UL141 variants disclosed herein. Any of the UL141 variants provided herein can be post-translationally modified in any manner recognized by those of skill in the art. Typical post-translational modifications for UL141 variants include interchain disulfide bonding and glycosylation. The post-translational modification can occur during production, in vivo, in vitro or otherwise. In some embodiments, the post-translational modification can be an intentional modification by a practitioner, for instance, using the methods provided herein.

Further included within the scope are UL141 variants fused to further peptides or polypeptides. Exemplary fusions include, but are not limited to, fusions to antibodies or to fragments thereof. In certain embodiments, the antibody is farletuzumab. In certain embodiments, the antibody is lexatumumab.

Additional fusions include UL141 variant fusions in which a methionine is linked to the N-terminus of the UL141 variant resulting from recombinant expression and fusions for the purpose of purification (including but not limited to, to poly-histidine or affinity epitopes). The UL141 variants may comprise protease cleavage sequences, polypeptide binding domains (including but not limited to, FLAG or poly-His) or other affinity based sequences (including but not limited to, FLAG, poly-His, GST, etc.).

In certain embodiments, the fusions support CAR-T or CAR-NK therapy. In certain embodiments, embodiments, the fusion comprises a UL-141 variant and one or more polypeptides useful for CAR-T therapy or CAR-NK therapy. In certain embodiments, the additional polypeptides are selected from any or all of CD28, IX40/4-1BB, and CD3-zeta, for instance as depicted in FIG. 15.

In particular embodiments, fusions according to SEQ ID NOS:34-65 are provided.

In certain embodiments, the one or more mutations increase activation of DR5 by the UL141 variant. In certain embodiments, one or more mutations increase activation of DR5 by the UL141 variant relative to wild-type UL141. In certain embodiments, one or more mutations increase activation of DR5 by the UL141 variant relative to a UL141 of the same sequence, other than the one or more mutations. In certain embodiments, one or more mutations increase activation of DR5 by the UL141 variant by 10%, 20%, 25%, 50%, 75%, 100%, 125%, 150%, 200%, 250%, 300%, 400%, 500%, 1000%, 2000%, 3000%, or more.

In certain embodiments, the one or more mutations increase binding of CD155 by the UL141 variant. In certain embodiments, one or more mutations increase binding of CD155 by the UL141 variant relative to wild-type UL141. In certain embodiments, one or more mutations increase binding of CD155 by the UL141 variant relative to a UL141 of the same sequence, other than the one or more mutations. In certain embodiments, one or more mutations increase binding of CD155 by the UL141 variant by 10%, 20%, 25%, 50%, 75%, 100%, 125%, 150%, 200%, 250%, 300%, 400%, 500%, 1000%, 2000%, 3000%, or more. In certain embodiments, the UL141 variant has increased activation of DR5 and increased binding of CD155.

In certain embodiments, the one or more mutations increase inhibition of CD155 by the UL141 variant. In certain embodiments, one or more mutations increase inhibition of CD155 by the UL141 variant relative to wild-type UL141. In certain embodiments, one or more mutations increase inhibition of CD155 by the UL141 variant relative to a UL141 of the same sequence, other than the one or more mutations. In certain embodiments, one or more mutations increase inhibition of CD155 by the UL141 variant by 10%, 20%, 25%, 50%, 75%, 100%, 125%, 150%, 200%, 250%, 300%, 400%, 500%, 1000%, 2000%, 3000%, or more. In certain embodiments, the UL141 variant has increased activation of DR5 and increased inhibition of CD155.

In certain embodiments, the one or more mutations increase inhibition of CD155-TIGIT activity by the UL141 variant. In certain embodiments, one or more mutations increase inhibition of CD155-TIGIT by the UL141 variant relative to wild-type UL141. In certain embodiments, one or more mutations increase inhibition of CD155-TIGIT by the UL141 variant relative to a UL141 of the same sequence, other than the one or more mutations. In certain embodiments, one or more mutations increase inhibition of CD155-TIGIT by the UL141 variant by 10%, 20%, 25%, 50%, 75%, 100%, 125%, 150%, 200%, 250%, 300%, 400%, 500%, 1000%, 2000%, 3000%, or more. In certain embodiments, the UL141 variant has increased activation of DR5 and increased inhibition of CD155-TIGIT activity.

In certain embodiments, the one or more mutations increase activation of T-cells by the UL141 variant. In certain embodiments, one or more mutations increase activation of T-cells by the UL141 variant relative to wild-type UL141. In certain embodiments, one or more mutations increase activation of T-cells by the UL141 variant relative to a UL141 of the same sequence, other than the one or more mutations. In certain embodiments, one or more mutations increase activation of T-cells by the UL141 variant by 10%, 20%, 25%, 50%, 75%, 100%, 125%, 150%, 200%, 250%, 300%, 400%, 500%, 1000%, 2000%, 3000%, or more. In certain embodiments, the UL141 variant has increased activation of DR5 and increased activation of T-cells.

### Bispecific and Multispecific Formats

In certain embodiments provided herein are bispecific or multispecific molecules or constructs comprising one or more UL141 variants described herein. The compounds comprise a first binding moiety according to a UL141 variant described herein. The compounds further comprise a second or further binding moiety specifically binding one or more further targets deemed useful to the person of skill. In certain embodiments, the further targets are selected from cancer antigens.

In certain embodiments, the further targets are selected from 4-1BB (CD137), 5′-nucleotidase, 5T4, activin receptor-like kinase 1, adenocarcinoma antigen, alpha-fetoprotein, angiopoietin 2, AXL, BAFF, BCMA, C242 antigen, CA-125, CA-IX, CEA, CCR4, CCR5, CD123, CD134, CD134, CD152, CD19, CD20, CD200, CD22, CD22, CD22, CD23, CD25, CD27, CD276, CD276, CD28, CD30, CD319, CD33, CD37, CD38, CD3E, CD40, CD44, CD51, CD51, CD56, CD70, CD70, CD74, CD79B, CD79B, CD80, CEACAMS, Claudin 18 Isoform 2, coagulation factor III, MCSF, CSF1R, CSF2, CTLA-4, CXCR4 (CD184), dendritic cell-associated lectin 2, DLL3, DLL4, DR5, EGFL7, EGFR, EGFR extracellular domain III, endoglin, EpCAM, EPHA3, EGFR, ERBB3 (HER3), FAP, FGFR2, fibronectin extra domain-B, folate hydrolase, folate receptor 1, Frizzled receptor, GD2 ganglioside, GD3 ganglioside, gelatinase B, glypican 3, GPNMB, GPRC5D, GUCY2C, HGF, HER1, HER2, HER2/neu, HGFR, histone complex, HLA-DR, human scatter factor receptor kinase, IGF-1 receptor (CD221), IGF-2, IL-13, IL-1α, IL-2, IL-3 receptor, IL-6, integrin α5β1, integrin αvβ, KIR2D, LAG3, Lewis-Y antigen, LIV-1, LRRC15, MIF, MCP-1, mesothelin, MST1R, MUC1, MUC5AC, nectin-4, NGNA ganglioside, Notch 1, Notch receptor, NRP1, PCDC1, PCDP1, PD-1, PDGFRA, PD-L1, phosphate-sodium co-transporter, phosphatidylserine, PTK7, root plate-specific spondin 3, ROR1, SDC1, SLAMF7, SLITRK6, STEAPI, TAG-72, TEM1, tenascin C, TIGIT, TRAIL-R1, TRAIL-R2, TROP-2, CTAA16.88, TAG-72, TWEAK receptor, TYRP1, VEGF-A, VEGFR-1, VEGFR2, and combinations thereof.

In certain embodiments, the second or further binding moiety targets tumor enriched receptors (such as FOLR1, MUC16, or EGFR). In certain embodiments, the second or further binding moiety targets tumor enriched immune check point receptors (such as PD-L1 or CD155). In certain embodiments, the second or further binding moiety targets T-cell (NK cell) enriched receptors such as PD-1, CTLA4, TIGIT, or LAG3.

The bispecific or multispecific molecules or constructs can be of any format apparent to the person of skill. The bispecific or multispecific molecules or constructs can be prepared according to standard techniques. The UL141 variant can be in any format deemed suitable. In certain embodiments, the UL141 variant is in the scFv format. The second or further binding moiety can be in any format deemed suitable. In certain embodiments, the second or further binding moiety is in the IgG1-Fc format.

### Vectors, Host Cells, and Recombinant Methods

Also provided are isolated nucleic acids encoding UL141 variants, vectors and host cells comprising the nucleic acids, and recombinant techniques for the production of the UL141 variants.

For recombinant production of the UL141 variants, the nucleic acid encoding it may be isolated and inserted into a replicable vector for further cloning (i.e., amplification of the DNA) or expression. In some aspects, the nucleic acid may be produced by homologous recombination, for example as described in U.S. Pat. No. 5,204,244.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615.

Illustrative examples of suitable host cells are provided below. these host cells are not meant to be limiting.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. lichenformis), Pseudomonas (P. aeruginosa), and Streptomyces. One useful E. coli cloning host is E. coli 294, although other strains such as E. coli B, E. coli X1776, and E. coli W3110 are suitable.

In addition to prokaryotes, eukaryotic microbes such as filamentous fungi or yeast are also suitable cloning or expression hosts for UL141 variant-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the UL141 variants may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 may be used.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the UL141 variants can be produced intracellularly, in the periplasmic space, or directly secreted into the medium. If the UL141 variant is produced intracellularly, as a first step, the particulate debris, either host cells or lysed fragments, is removed, for example, by centrifugation or ultrafiltration. For example, Carter et al. (Bio/Technology, 1992, 10:163-167) describes a procedure for isolating polypeptides which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

The UL141 variants can be purified according to standard techniques known to those of skill in the art.

### Pharmaceutical Compositions and Methods of Administration

The UL141 variants provided herein can be formulated into pharmaceutical compositions using methods available in the art and those disclosed herein. Any of the UL141 variants provided herein can be provided in the appropriate pharmaceutical composition and be administered by a suitable route of administration.

The methods provided herein encompass administering pharmaceutical compositions comprising at least one UL141 variant provided herein and one or more compatible and pharmaceutically acceptable carriers. In this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in Martin, E. W., Remington's Pharmaceutical Sciences.

In clinical practice the pharmaceutical compositions or UL141 variants provided herein may be administered by any route known in the art. In certain embodiments, a pharmaceutical composition or UL141 variant provided herein is administered parenterally.

The compositions for parenteral administration can be emulsions or sterile solutions. Parenteral compositions may include, for example, propylene glycol, polyethylene glycol, vegetable oils, and injectable organic esters (e.g., ethyl oleate). These compositions can also contain wetting, isotonizing, emulsifying, dispersing and stabilizing agents. Sterilization can be carried out in several ways, for example using a bacteriological filter, by radiation or by heating. Parenteral compositions can also be prepared in the form of sterile solid compositions which can be dissolved at the time of use in sterile water or any other injectable sterile medium.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic UL141 variants.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific UL141 variant in the dosage form. The composition or single unit dosage form, if desired, can also contain minor amounts of wetting or emulsifying agents, or pH buffering agents.

Lactose free compositions provided herein can comprise excipients that are well known in the art and are listed, for example, in the U.S. Pharmacopeia (USP) SP (XXI)/NF (XVI). In general, lactose free compositions comprise an active ingredient, a binder/filler, and a lubricant in pharmaceutically compatible and pharmaceutically acceptable amounts. Exemplary lactose free dosage forms comprise an active ingredient, microcrystalline cellulose, pre gelatinized starch, and magnesium stearate.

Components of the pharmaceutical composition can be supplied either separately or mixed together in unit dosage form, for example, as a dry lyophilized powder or water free concentrate. Where the composition is to be administered by infusion, it can be dispensed with an infusion bottle containing sterile pharmaceutical grade water or saline. Where the composition is administered by injection, an ample of sterile water for injection or saline can be provided so that the ingredients may be mixed prior to administration.

In some embodiments, the pharmaceutical composition is supplied as a dry sterilized lyophilized powder that is capable of being reconstituted to the appropriate concentration for administration to a subject. In some embodiments, UL141 variants are supplied as a water free concentrate.

In another embodiment, the pharmaceutical composition is supplied in liquid form. In some embodiments, the pharmaceutical composition is provided in liquid form and is substantially free of surfactants and/or inorganic salts.

In some embodiments, the pharmaceutical composition is formulated as a salt form. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Further encompassed herein are anhydrous pharmaceutical compositions and dosage forms comprising a UL141 variant, since water can facilitate the degradation of some UL141 variants.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Further provided are pharmaceutical compositions and dosage forms that comprise one or more excipients that reduce the rate by which a UL141 variant will decompose. Such excipients, which are referred to herein as “stabilizers,” include, but are not limited to, antioxidants such as ascorbic acid, pH buffers, or salt buffers.

### Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the UL141 variants disclosed herein can also be incorporated into the parenteral dosage forms.

### Dosage and Unit Dosage Forms

In human therapeutics, the doctor will determine the posology which he considers most appropriate according to a preventive or curative treatment and according to the age, weight, stage of the infection and other factors specific to the subject to be treated.

The amount of the UL141 variant or composition which will be effective in the prevention or treatment of a disorder or one or more symptoms thereof will vary with the nature and severity of the disease or condition, and the route by which the UL141 variant is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

The dose can be administered according to a suitable schedule, for example, once, two times, three times, or for times weekly. It may be necessary to use dosages of the UL141 variant outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the UL141 variant provided herein are also encompassed by the herein described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of a UL141 variant or composition provided herein followed by one or more maintenance doses.

In certain embodiments, a dose of a UL141 variant or composition provided herein can be administered to achieve a steady-state concentration of the UL141 variant in blood or serum of the subject. The steady-state concentration can be determined by measurement according to techniques available to those of skill or can be based on the physical characteristics of the subject such as height, weight and age.

### Therapeutic Applications

For therapeutic applications, UL141 variants disclosed herein are administered to a mammal, generally a human, in a pharmaceutically acceptable dosage form such as those known in the art and those discussed above. For example, the UL141 variants disclosed herein may be administered to a human intravenously as a bolus or by continuous infusion over a period of time, by intravenous, intramuscular, intraperitoneal, intra-cerebrospinal, subcutaneous, intra-articular, intrasynovial, intrathecal, or intratumoral routes. The UL141 variants also are suitably administered by peritumoral, intralesional, or perilesional routes, to exert local as well as systemic therapeutic effects.

A therapeutically effective amount of the UL141 variant or composition is an amount that is effective to reduce the severity, the duration and/or the symptoms of a particular disease or condition. The amount of the UL141 variant or composition that will be therapeutically effective in the prevention, management, treatment and/or amelioration of a particular disease can be determined by standard clinical techniques. The precise amount of the UL141 variant or composition to be administered with depend, in part, on the route of administration, the seriousness of the particular disease or condition, and should be decided according to the judgment of the practitioner and each subject's circumstances.

The UL141 variants and compositions can be used to treat or prevent any disease or condition deemed suitable by the practitioner of skill. In certain embodiments, the UL141 variants and compositions can be used to treat cancer. In certain embodiments, the cancer is a solid tumor. In certain embodiments, the cancer is selected from the group consisting of bone and muscle sarcomas, brain and nervous system cancer, breast cancer, endocrine cancer, eye cancer, gastrointestinal cancer, genitourinary and gynecologic cancer, head and neck cancer, skin cancer, thoracic and respiratory cancer, and HIV/AIDS related cancer. In certain embodiments, the cancer is selected from chondrosarcoma; Ewing's sarcoma; malignant fibrous histiocytoma of bone/osteosarcoma; osteosarcoma; rhabdomyosarcoma; leiomyosarcoma; myxosarcoma; fibrocartilaginous mesenchymoma of bone; astrocytoma; brainstem glioma; pilocytic astrocytoma; ependymoma; primitive neuroectodermal tumor; cerebellar astrocytoma; cerebral astrocytoma; glioblastoma; glioma; medulloblastoma; neuroblastoma; oligodendroglioma; pineal astrocytoma; pituitary adenoma; visual pathway and hypothalamic glioma; breast cancer; inflammatory breast cancer; invasive lobular carcinoma; tubular carcinoma; invasive cribriform carcinoma of the breast (also termed invasive cribriform carcinoma); medullary carcinoma; male breast cancer; phyllodes tumor; mammary secretory carcinoma; papillary carcinomas of the breast; adrenocortical carcinoma; islet cell carcinoma (endocrine pancreas); multiple endocrine neoplasia syndrome; parathyroid cancer; pheochromocytoma; thyroid cancer; merkel cell carcinoma; uveal melanoma; retinoblastoma; optic nerve glioma; anal cancer; appendix cancer; cholangiocarcinoma; carcinoid tumor, gastrointestinal; colon cancer; extrahepatic bile duct cancer; gallbladder cancer; gastric (stomach) cancer; gastrointestinal carcinoid tumor; gastrointestinal stromal tumor (GIST); hepatocellular cancer; pancreatic cancer, islet cell; rectal cancer; small intestine cancer; bladder cancer; cervical cancer; endometrial cancer; extragonadal germ cell tumor; ovarian cancer; ovarian epithelial cancer (surface epithelial-stromal tumor); ovarian germ cell tumor; penile cancer; kidney cancer; renal cell carcinoma; renal pelvis and ureter, transitional cell cancer; prostate cancer; testicular cancer; gestational trophoblastic tumor; ureter and renal pelvis, transitional cell cancer; urethral cancer; uterine sarcoma; vaginal cancer; vulvar cancer; Wilms tumor (nephroblastoma); esophageal cancer; head and neck cancer; nasopharyngeal carcinoma; oral cancer; oropharyngeal cancer; paranasal sinus and nasal cavity cancer; pharyngeal cancer; salivary gland cancer; hypopharyngeal cancer; basal cell carcinoma; squamous cell carcinoma; squamous cell skin cancer; skin adnexal tumors (e.g. sebaceous carcinoma); melanoma; merkel cell carcinoma; keratoacanthoma; sarcomas of primary cutaneous origin (e.g. dermatofibrosarcoma protuberans); lymphomas of primary cutaneous origin (e.g. mycosis fungoides); adenocarcinoma of the lung; bronchial adenomas/carcinoids; small cell lung cancer; mesothelioma; non-small cell lung cancer; non-small cell lung carcinoma; pleuropulmonary blastoma; laryngeal cancer; thymoma and thymic carcinoma; squamous-cell carcinoma of the lung; AIDS-related cancers; Kaposi sarcoma; epithelioid hemangioendothelioma (EHE); desmoplastic small round cell tumor; liposarcoma; and combinations thereof.

### Combination Therapy

In certain embodiments, provided herein are methods of treatment that comprise administration of a UL141 variant or fusion or bispecific or multispecific compound or molecule disclose herein in combination with one or more further agents useful for treating a condition or disorder deemed useful to the person of skill. In certain embodiments, the further agent is selected from cancer therapeutics deemed useful by the person of skill. In certain embodiments, the further agent targets PD-1.

In certain embodiments, the further agent is a cancer therapeutic. In certain embodiments, the further agents are selected from ABVD; AC; ATO; ATRA; Abemaciclib (Verzenois); Abiraterone (Zytiga); Abraxane; Abstral; Acalabrutinib; Actimorph; Actinomycin D; Actiq; Adriamycin; Afatinib (Giotrif); Afinitor; Aldara; Aldesleukin (IL-2, Proleukin or interleukin 2); Alectinib; Alectinib (Alecensa); Alemtuzumab (Campath, MabCampath); Alkeran; Amsacrine (Amsidine, m-AMSA); Amsidine; Anastrazole (Arimidex); Apalutamide; Ara C; Arimidex; Aromasin; Arsenic trioxide (Trisenox, ATO); Asparaginase (Spectrila, Erwinase, Oncaspar); Atezolizumab; Avelumab; Axitinib (Inlyta); Azacitidine (Vidaza, Onureg); BEACOPP; BEAM; Bendamustine (Levact); Besponsa; Bevacizumab (Avastin); Bexarotene (Targretin); Bicalutamide (Casodex); Bleomycin; Bleomycin, etoposide and platinum (BEP); Blinatumomab (Blincyto); Bortezomib (Velcade); Bortezomib thalidomide and dexamethasone (VTD); Bortezomib, cyclophosphamide and dexamethasone (VCD); Bortezomib, melphalan and prednisalone (VMP); Bosulif; Bosutinib (Bosulif); Brentuximab; Brigatinib (Alunbrig); Buserelin (Suprefact); Busulfan; CAPE-OX; CAPOX; CAV; CCNU; CHOP; Cabazitaxel (Jevtana); Cabometyx; Cabozantinib (Cometriq, Cabometyx); Caelyx; Calpol; Campto; Capecitabine (Xeloda); Caprelsa; CarboTaxol; Carboplatin; Carboplatin and etoposide; Carboplatin and paclitaxel; Carfilzomib and dexamethasone; Carmustine (BCNU); Casodex; Cemiplimab; Ceritinib (Zykadia); Cetuximab (Erbitux); Chlorambucil (Leukeran); Cisplatin; Cisplatin and capecitabine (CX); Cisplatin and fluorouracil (5FU); Cisplatin, etoposide and ifosfamide (VIP); Cisplatin, fluorouracil (5FU) and trastuzumab; Cladribine; Clasteon; Co-codamol (Kapake, Solpadol, Tylex); Cometriq; Cosmegen; Crisantaspase; Crizotinib (Xalkori); Cyclophosphamide; Cyclophosphamide, thalidomide and dexamethasone (CTD); Cyprostat; Cyproterone acetate; Cytarabine (Ara C, cytosine arabinoside); Cytarabine into spinal fluid (intrathecal cytarabine); Cytosine arabinoside; DHAP; DTIC; Dabrafenib (Tafinlar); Dabrafenib and trametinib; Dacarbazine (DTIC); Dacomitinib; Dactinomycin (actinomycin D); Daratumumab; Darolutamide (Nubega); Darzalex; Dasatinib (Sprycel); Daunorubicin; Daunorubicin, cytarabine and midostaurin; Decapeptyl SR; Degarelix (Firmagon); Denosumab (Prolia, Xgeva); Dexamethasone; Diamorphine; Disodium pamidronate; Disprol; Docetaxel (Taxotere); Docetaxel, cisplatin and fluorouracil (TPF); Doxifos; Doxorubicin (Adriamycin); Doxorubicin and ifosfamide; Durogesic; Durvalumab (Imfinzi); EC; ECF; EOF; EOX; EP (Etoposide and cisplatin); ESHAP; Effentora; Encorafenib and binimetinib; Encorafenib and cetuximab; Entrectinib (Rozlytrek); Enzalutamide; Epirubicin; Epirubicin cisplatin and capecitabine (ECX); Epirubicin, carboplatin and capecitabine (ECarboX); Erbitux; Eribulin (Halaven); Erlotinib (Tarceva); Erwinase; Etopophos; Etoposide (Etopophos); Everolimus (Afinitor); Evoltra; Exemestane (Aromasin); FOLFIRINOX; FOLFOX; FOLFOXIRI; Faslodex; Femara; Fentanyl; Firmagon; Fludara; Fludarabine (Fludara); Fludarabine, cyclophosphamide and rituximab (FCR); Fluorouracil (5FU); Fluorouracil (5FU) and mitomycin C; Fluorouracil, Leucovorin, Oxaliplatin and Docetaxel (FLOT); Flutamide; Folinic acid, fluorouracil and irinotecan (FOLFIRI); Fotivda; Fulvestrant (faslodex); G-CSF; Gefitinib (Iressa); GemCarbo (gemcitabine and carboplatin); GemTaxol; Gemcitabine (Gemzar); Gemcitabine and capecitabine (GemCap); Gemcitabine and cisplatin (GC); Gemcitabine and nab-paclitaxel; Gemcitabine and paclitaxel (GemTaxol); Gemtuzumab ozogamicin, daunorubicin and cytarabine; Gemzar; Giotrif, Gliadel (carmustine wafers); Glivec; Gonapeptyl Depot; Goserelin (Zoladex) for breast cancer; Goserelin for prostate cancer; Granulocyte colony stimulating factor (G-CSF); Halaven; Herceptin; Herzuma; Hycamtin; Hydrea; Hydrocortisone; Hydroxycarbamide (Hydrea); Hydroxyurea; ICE; IL-2; IPE; Ibandronic acid (Bondronat); Ibrance; Ibrutinib (Imbruvica); Ibuprofen; Iclusig; Idarubicin; Ifosfamide (Mitoxana); Imatinib (Glivec); Imiquimod cream (Aldara); Inotuzumab ozogamicin; Interleukin; Ipilimumab (Yervoy); Ipilimumab and nivolumab; IrCap; Iressa; Irinotecan (Campto); Irinotecan and capecitabine (Xeliri); Irinotecan de Gramont; Irinotecan modified de Gramont; Ixazomib lenalidomide and dexamethasone; Jevtana; Kadcyla; Kapake; Keytruda; Kisqali; Lanreotide (Somatuline); Larotrectinib (Vitrakvi); Lenalidomide (Revlimid); Lenvatinib; Letrozole (Femara); Leukeran; Leuprorelin; Leustat; Levact; Liposomal doxorubicin; Litak; Lomustine; Lonsurf: Lorlatinib (Lorviqua); Lutrate; Lynparza; Lysodren; MAP; MMM; MPT; MST Continus; MVAC; MXL; MabCampath; Mabthera; Medroxyprogesterone acetate (Provera); Megace; Megestrol acetate (Megace); Melphalan (Alkeran); Mepact; Mercaptopurine (Xaluprine); Methotrexate; Methylprednisolone; Mifamurtide; Mitomycin C; Mitotane (Lysodren); Mitoxana; Mitoxantrone (Mitozantrone); Mobocertinib; Mobocertinib (Exkivity); Modified de Gramont; Morphgesic SR; Morphine; m-AMSA; Nab paclitaxel (Abraxane); Navelbine; Nelarabine (Atriance); Neratinib (Nerlynx); Nerlynx; Nexavar; Nilotinib (Tasigna); Nintedanib; Nipent; Niraparib (Zejula); Nivolumab (Opdivo); Obinutuzumab (Gazyvaro); Octreotide; Olaparib (Lynparza); Ontruzant; Onureg; Opdivo; Oramorph; Osimertinib (Tagrisso); OxCap; Oxaliplatin; Oxaliplatin and capecitabine (Xelox); PC; PE; PMitCEBO; POMB/ACE; Paclitaxel (Taxol); Paclitaxel and carboplatin (PC, CarboTaxol); Palbociclib (Ibrance); Pamidronate; Panadol; Panitumumab (Vectibix); Panobinostat, bortezomib and dexamethasone; Paracetamol; Pazopanib (Votrient); Peginterferon alfa 2a; Pembrolizumab (Keytruda); Pemetrexed (Alimta); Pemetrexed and carboplatin; Pemetrexed and cisplatin; Pemigatinib; Pentostatin (Nipent); Perjeta; Pertuzumab (Perjeta); Polatuzumab vedotin, bendamustine and rituximab (Pola-BR); Pomalidomide and dexamethasone; Ponatinib; Prednisolone; Procarbazine; Procarbazine, lomustine and vincristine (PCV); Proleukin; Prolia; Prostap; Provera; R-CHOP; R-CVP; R-DHAP; R-ESHAP; R-GCVP; R-Idelalisib (Zydelig); RICE; Raloxifene; Raltitrexed (Tomudex); Regorafenib (Stivarga); Revlimid; Ribociclib (Kisqali); Rituximab; Rixathon; Rubraca; Rucaparib (Rubraca); Ruxience; Ruxolitinib; Selpercatinib; Sevredol; Sodium clodronate (Clasteon, Loron); Solpadol; Sorafenib; Steroids (dexamethasone, prednisolone, methylprednisolone and hydrocortisone); Stivarga; Streptozocin (Zanosar); Sunitinib (Sutent); Sutent; TIP; Tafinlar; Tagrisso; Talimogene laherparepvec (T-VEC); Tamoxifen; Tarceva; Targretin; Tasigna; Taxol; Taxotere; Taxotere and cyclophosphamide (TC); Tecentriq; Temodal; Temozolomide (Temodal); Tepadina; Tepotinib; Thalidomide; Thiotepa (Tepadina); Tivozanib (Fotivda); Tomudex; Topotecan (Hycamtin); Trabectedin (Yondelis); Trastuzumab (Herceptin); Trastuzumab and pertuzumab; Trastuzumab emtansine (Kadcyla); Treosulfan; Tretinoin (Vesanoid, ATRA); Trifluridine and tipiracil (Lonsurf); Triptorelin; Trisenox; Truxima; Tucatinib, trastuzumab and capecitabine; Tylex; VDC/IE; VIDE; Vargatef; VeIP; Vectibix; Velcade; Vemurafenib (Zelboraf); Venetoclax (Venclyxto); Vesanoid; Vidaza; Vinblastine; Vincristine; Vincristine, actinomycin D and cyclophosphamide (VAC); Vincristine, actinomycin D and ifosfamide (VAI); Vinorelbine (Navelbine); Votrient; XELOX; Xalkori; Xeloda; Xgeva; Xtandi; Yervoy; Yondelis; Zanosar; Zelboraf; Zoladex (breast cancer); Zoladex (prostate cancer); Zoledronic acid (Zometa); Zometa; Zomorph; Zydelig; Zytiga; and combinations thereof.

In certain embodiments, the further agents are selected from pembrolizumab, nivolumab, cemiplimab, dostalimab, retifanlimab, vopratelimab, spatalizumab, camrelizumab, sintilimab, tislelizumab, toripalimab, MGA012, AMP-224, AMP-514, acrixolimab, and combinations thereof. In certain embodiments, the further agents are selected from atezolizumab, avelumab, durvalumab, KN035, cosibelimab, AUNP12, CA-170, BMS-986189, and combinations thereof.

### Diagnostic Applications

In some embodiments, the UL141 variants provided herein are used in diagnostic applications. For example, a UL141 variant disclosed herein that is specific for a given receptor may be useful in assays for the given receptor. In some aspects the UL141 variant can be used to detect the expression of DR5 in various cells and tissues. In some aspects the UL141 variant can be used to detect the expression of CD155 in various cells and tissues. These assays may be useful, for example, diagnosing cancer.

In the methods, the formation of a complex between the UL141 variant and receptor can be detected by any method known to those of skill in the art. Examples include assays that use secondary reagents for detection, ELISA's and immunoprecipitation and agglutination assays. A detailed description of these assays is, for example, given in Harlow and Lane, Antibodies: A Laboratory Manual (Cold Spring Harbor Laboratory, New York 1988 555-612, WO 96/13590 to Maertens and Stuyver, Zrein et al. (1998) and WO 96/29605.

For in situ diagnosis, the UL141 variant may be administered to a subject by methods known in the art such as, for example, intravenous, intranasal, intraperitoneal, intracerebral, intraarterial injection such that a specific binding between the UL141 variant and receptor may occur. The UL141 variant/receptor complex may conveniently be detected through a label attached to the UL141 variant or any other art-known method of detection.

In some diagnostic applications, the UL141 variant may be labeled with a detectable moiety. Suitable detectable moieties include, but are not limited to radioisotopes, fluorescent labels, and enzyme-substrate labels.

### Kits

In some embodiments, a UL141 variant as described herein can be provided in a kit, i.e., a packaged combination of reagents in predetermined amounts with instructions for performing a procedure. In some embodiments, the procedure is a diagnostic assay. In other embodiments, the procedure is a therapeutic procedure.

### EXAMPLES

### Example 1

### Design and Expression of UL141 Variants

For this disclosure, ovarian cancer was selected as a model tumor. Ovarian tumors are known to be most immune desert compared to other solid tumors. Hence, when a DR5 targeting therapeutic works in ovarian tumors, this supports efficacy in other solid tumors as long as those tumors express DR5.

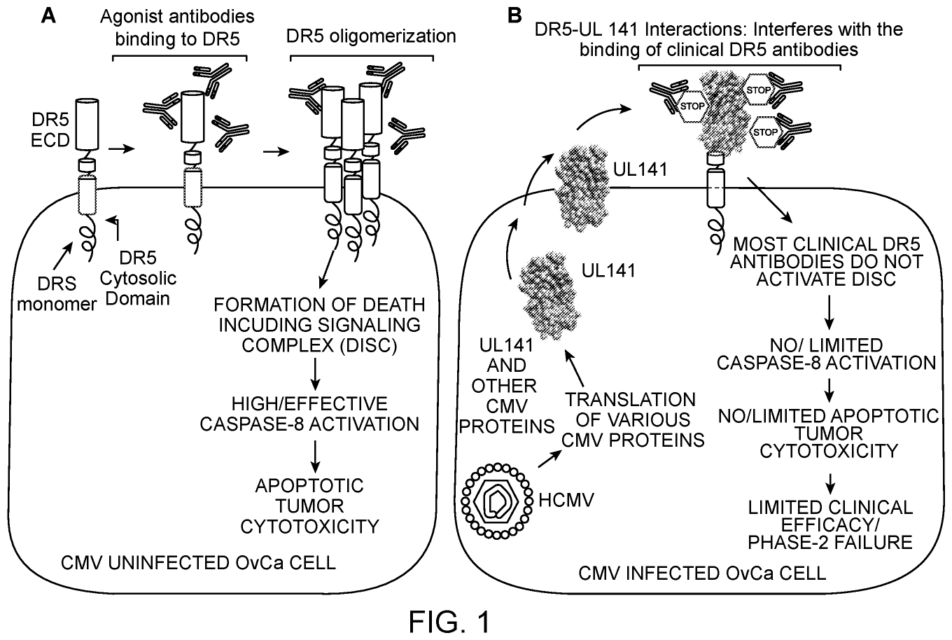

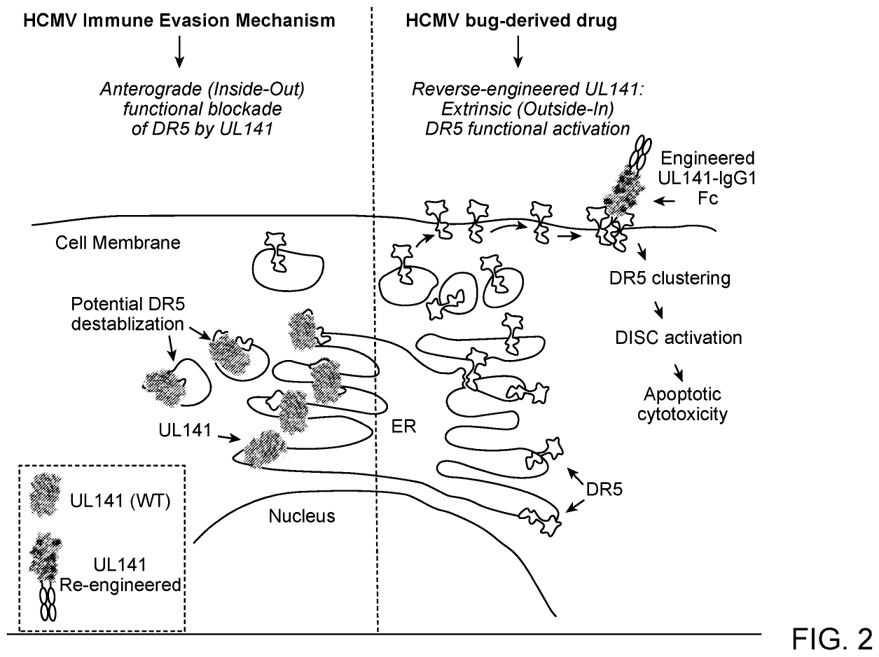

The wild-type human cytomegalovirus UL141 protein destabilizes and blocks DR5 oligomerization in infected cells. FIG. 1 provides HCMV UL141 protein-mediated destabilization and blockade of DR5 oligomerization in infected OvCa cells. (A) Healthy OvCa and stroma cells (uninfected or hCMV negative) contain DR5 monomer not bound to UL141. Hence, DR5 is oligomerized by various clinical agonist antibodies, which results in the activation of cytotoxic apoptotic signaling selectively in OvCa cells, a working mechanism for the clinical function of DR5 agonist antibodies. (B) Upon hCMV infection, viral UL141 protein binds to DR5 to destabilize protein levels in the ER-Golgi network and the cell surface. Altogether, the latter significantly limits the surface DR5 accessibility to be targeted by clinical DR5 agonist antibodies. The UL141 mediated DR5 epitope burial could potentially inhibit or limited DR5 oligomerization by clinical antibodies. Collectively hCMV infection and UL141 protein could interfere with clinical efficacy in OvCa.

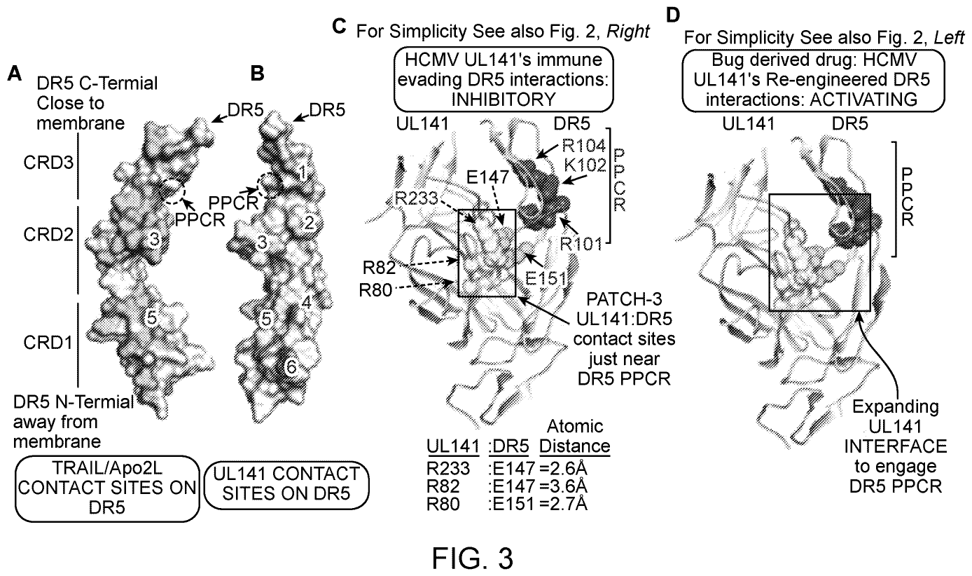

In the present examples, UL141 is evolved and designed to provide a therapeutic that activates DR5. FIG. 2 provides a working model of hCMV bug derived drug: recombinant UL141 variant IgG1 Fc. (Left) In hCMV infected OvCa cells, UL141 saturates DR5 during anterograde (inside-out) transport in ER, destabilizing overall surface levels of DR5. The latter is hCMV immune evasion mechanism to avoid elimination by innate NK cells expressing DR5 ligand, Apo2L. (Right) The UL141 variant IgG1-Fc if reverse engineered with high affinity interactions of engaging and extrinsically (outside-in) activating DR5 would be an effective hCMV bug derived drug for OvCa. In addition, recombinant UL141 variant have been engineered with added capacity to block CD155-TIGIT negative immune checkpoint interactions to maintain T-cell activation in ovarian tumors.

The present examples modulate high-affinity DR5-UL141 interactions via the reverse engineering of UL141 to activate extrinsic (outside-in) DR5 clustering, signaling, and apoptotic signaling (FIG. 2, right). The proposed DR5 activating hypothesis of reverse engineering of UL141 is based on engagement of a crucial patch of positively charged (PPCR) DR5 residues being the most effective DR5 activating epitope. UL141-DR5 interactions suggest no meeting of PPCR by UL141, despite engaging residues just above PPCR (FIG. 3C, red PPCR put side the blue square).

FIG. 3 provides PPCR epitope remains unengaged by HCMV UL141 protein binding to DR5 (A-B) Comparison of Apo2L (DR5 ligand) and UL141 binding footprints based on DR5 based on crystal structure study, PDB:419X. The cyan color in (A) depicts Apo2L and yellow color in (B) depicts UL141 binding DR5 epitopes. (C) The inhibitory and destabilizing UL141 interaction against DR5 patch-3 is mediated by UL141's positively charged residues (arginine: R80, R82, R233, shown in yellow). The three arginine residues of UL141 form multiple salt-bridge formations with negatively charged glutamates (E147 and E151, shown in cyan color) on DR5. Importantly, despite being so close, the DR5 patch of positively charged residues named PPCR (R101, K102, R104, shown in red is not engaged by UL141. (D) We have reverse engineered and affinity matured UL141 IgG1-Fc (Lead named UL141^{D11}) which engages PPCR to activate DR5 and extrinsic apoptosis of ovarian cancer cells. On top of that UL141 is further engineered to inhibit CD155-TIGIT immune inhibitory interactions.

Thus, reverse engineering and affinity mature mutagenesis capable of engaging the UL141 interface with DR5 PPCR would generate an effective DR5 activating protein (FIG. 3D, blue square extended to engage red PPCR).

Mutants were created by comprehensive site directed mutation first in region from 215-245 amino acid of UL141 alongside of lock and key regions. Various UL141 mutation were ordered and synthesized as Genestring (life technologies) with flanking primers to our antibody cloning vectors to combine with IgG1 and IgG4 Fc. The received DNA sequences were PCR amplified followed. DNA was gel purified and inserted into pcDNA 3.1⁺ vector (CMV promoter) by making use of In-Fusion HD Cloning Kits (Takara Bio). EcoRI and HindIII digested vector was incubated with overlapping PCR fragments of various recombinant mutant DNAs, with infusion enzyme (1:2, vector: insert ratio) at 55 °C for 30 min, followed by additional 30 min incubation on ice after adding E. coli Stellar™ cells (Clontech). Transformation and bacterial screening were carried out using standard cloning methods. Positive clones were sequence confirmed in a 3-tier method. Confirmed bacterial colonies were Sanger sequenced upon PCR followed by re-sequencing of mini-prep DNA extracted from the positive colonies. Finally, maxiprep was re-sequenced prior to each transfection. All sequences of confirmed UL141 clones were expressed using CHO cells.

Free style CHO-S cells (Invitrogen) were cultured and maintained according to supplier's recommendations (Life technologies) biologics using free style CHO expression media (life technologies) and as previously published by us. A ratio of 1:2 (light chain, VL: heavy chain, VH) DNA was transfected using 1 g/ml polyethylenimine (PEI). After transfection, cells were kept at 37 °C for 24 hr. After 24 hr, transfected cells were shifted to 32 °C to slow down the growth for 9 additional days. Cells were routinely fed (every 2^{nd} day) with 1:1 ratio of tryptone feed and CHO Feed B. After 10 days, supernatant from cultures was harvested and antibodies were purified using protein-A affinity columns. The detailed amino acid of all recombinant molecules is provided. All molecules were tested for cell killing activity side by side. The molecule showing the highest tumor cell cytotoxicity against DR5+ cells were further comprehensive site directed mutated against the Lock and Key region followed by cloning and expression as described earlier.

Various transfected UL141 mutants' constructs were affinity purified using HiTrap MabSelect SuRe (GE, 11003493) protein-A columns. Transfected cultures were harvested after 10 days and filtered through 0.2-micron PES membrane filters (Milipore Express Plus). Cleaning-in-place (CIP) was performed for each column using 0.2 M NaOH wash (20 min). Following cleaning, columns were washed 3 times with Binding buffer (20 mM sodium phosphate, 0.15 M NaCl, pH 7.2). Filtered supernatant containing recombinant antibodies or antigens were passed through the columns at 4 °C. Prior to elution in 0.1 M sodium citrate, pH 3.0-3.6, the columns were washed 3 times with binding buffer (pH 7.0). The pH of eluted antibodies/UL141 mutants was immediately neutralized using sodium acetate (3 M, pH 9.0). After protein measurements at 280 nm, antibodies were dialyzed in PBS using Slide-A-Lyzer 3.5K (Thermo Scientific, 66330). Antibodies/UL141 mutants were run on the gel to confirm the size followed by testing in cytotoxicity and other assays.

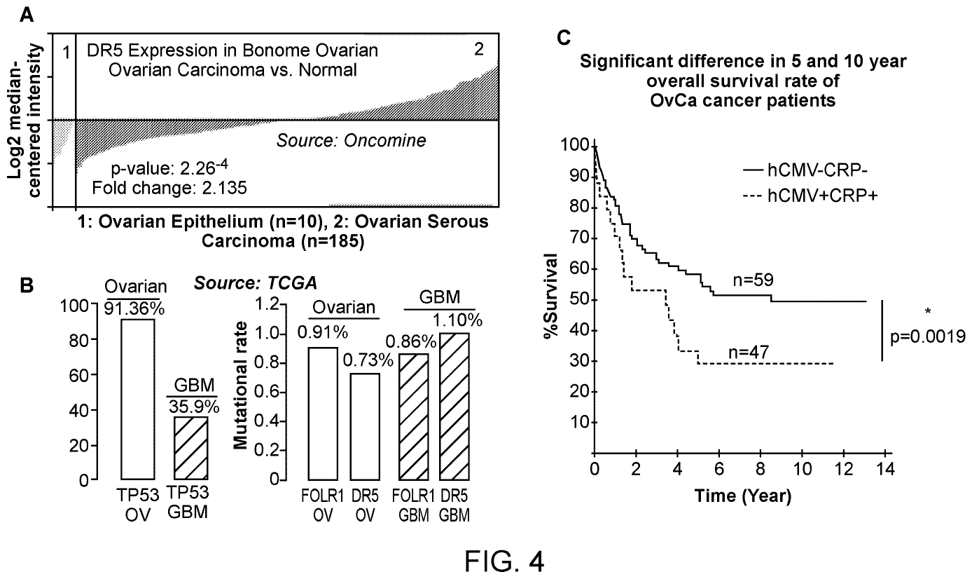

FIG. 4 provides higher DR5 profile in OvCa and inverse relation of HCMV infection in OvCa. A) More than 50% of 185 human primary & recurrent ovarian tumors overexpress elevated levels of DR5 (TNFRSF10B, Affymetrix U133A array). In remaining ~50% of tumor samples, DR5 levels were not down regulated beyond the normal ovarian epithelium (1 vs 2). B-left) More than 91% of OvCa (Red bars) carry p53 mutations compared to 36% of GBM (blue bars). B-right) DR5 have mutational rate of <0.73%, which is lower compared to therapeutically tested FOLR1<91% in OvCa. Data reflects increased applicability of DR5 therapeutics toward a large patient population. (C) Kaplan-Meier curve showing relapse-free survival in hCMV CRP protein-positive human ovarian tumors (n=59, 47). P-values calculated by Chi-square analysis.

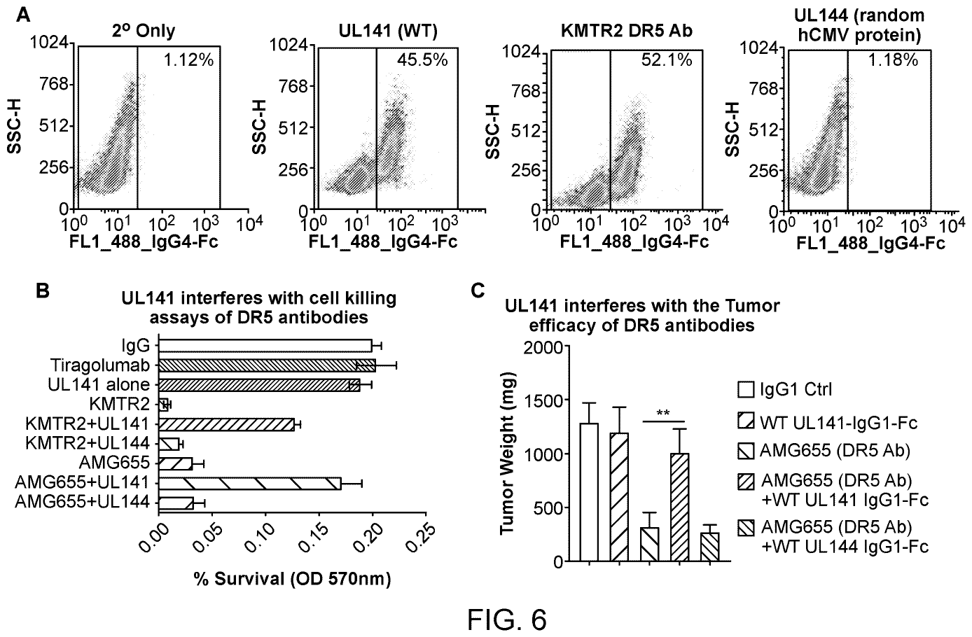

To test if the original viral UL141 (named UL141 WT hereafter) saturates DR5 extracellularly, recombinant UL141 was expressed as a His-tag protein using described bacterial expression (Shivange et al., 2018, Cancer Cell. 2018; 34(2):331-45). Side by side, UL144 was expressed as a control protein. Next, binding to DR5 was confirmed by flow cytometry, which showed high DR5 saturation similar to DR5 antibody KMTR2 (FIG. 6a). Next, we tested if WT UL141 will inhibit the cell-killing function of clinical DR5 antibodies. Preliminary, we observed high inhibition of ovarian tumor cell killing by UL141 WT, supporting UL141 binding to DR5 being directly responsible for the binding and tumor-killing activity of various clinical DR5 against (FIG. 6b). We confirmed these results using ovarian tumor grafts (OVCAR-3 grafts). Almost complete reversal of tumor efficacy was observed after the addition of UL141 WT (FIG. 6c). These results collectively point toward one additional possibility of clinically tested DR5 agonist failure in hCMV infected patients.

FIG. 6 provides HCMV UL141 (but not UL144) interferes with apoptotic activity of clinical DR5 antibodies. (A) Flow cytometry confirming binding of WT UL141 to DR5 on surface of cancer cells. (B) OVCAR-3 cells were treated with indicated antibodies either alone or after 2 hours prior treatment of UL141 or UL144. Only UL141 but (UL144) preincubation inferred with the cell survival. (C) OVCAR-3 grafted tumors were treated with DR5 agonist AMG655 alone or with AMG655 along with competing UL141 (and control UL144) as indicated. Harvested tumor weights were measured after 4 weeks.

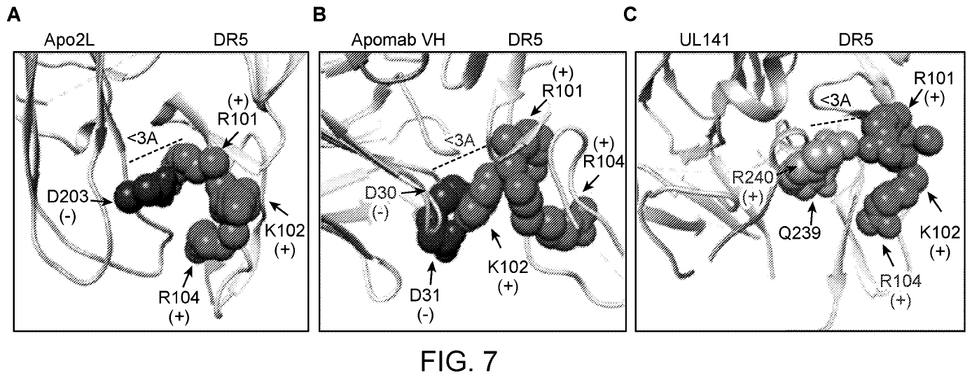

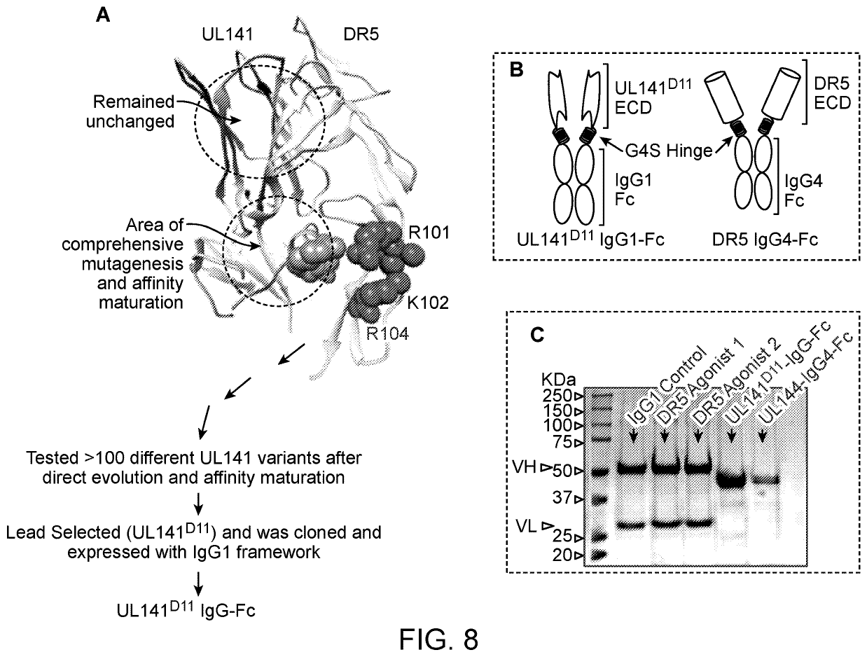

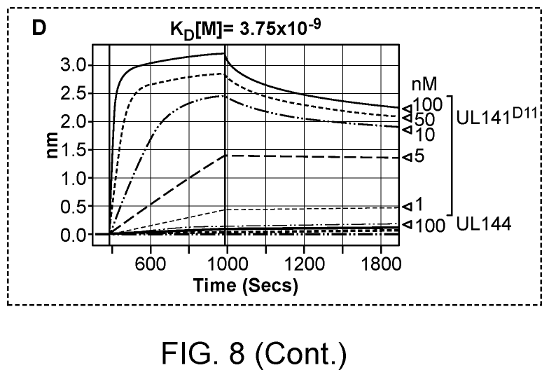

A critical negative apoptotic regulatory patch of positive charge residue (PPCR) epitope is in the CRD3 domain of DR5, which is vital for efficient DR5 activation and tumor cytotoxicity by agonist antibodies. UL141-DR5 interactions were analyzed at the crystal structure level. Unlike DR5 activating Apo2L ligand or agonist antibody apomab, both of which form salt bridges via their negatively charged aspartate (D) or glutamate (E) residues with DR5 arginine (R) or lysine (K) of PPCR motif, UL141 interface (near DR5 PPCR motif) contains sterically repelling positively charged arginine motif (less than 3 Å away from PPCR) (FIG. 7). By comprehensive mutagenesis and direct evolution (supported with affinity maturation) in this region of UL141 (FIG. 8a), there were reverse re-engineered negatively charged residues and additional stabilizing framework mutations in this steric DR5 PPCR repelling part of UL141 to form salt bridges with PPCR. A series of (>100) variants have been tested in binding and killing assays. Among these UL141 variants, the selected lead was cloned into the IgG1 framework named UL141 IgG1-Fc variant: UL141^{D11} (FIG. 8b).

FIG. 7 provides unlike DR5 activating ligand Apo2L and antibody Apomab, DR5 destabilizing hCMV UL141 contain positive charge residues at DR5 PPCR interface. (A) PDB:ID0G, (B) PDB:40D2, (C) PDB:419X shows space-filling diagrams of Apo2L:DR5, Apomab:DR5 and UL141:DR5 interface near PPCR region of DR5. For these images, a sphere is drawn around each atom, showing the relative size of the atom. In case of Apo2L:DR5 (A) and Apomab:DR5 (B) negatively charged residues (blue) forms highly significant (<3 Å apart) salt bridges with positively charged PPCR residues (Red) to activate DR5 signaling (R). Only UL141 (C) contains repelling positive charged (gold) residues at DR5 PPCR interface (Red) and does not activate DR5.

FIG. 8 provides reverse engineering of hCMV derived UL141 for an extrinsic (outside-in) DR5 apoptotic activator. (A) Area of UL141 (blue circle) that underwent directed evolution and affinity maturation in a comprehensive screen to select the lead molecule named UL141^{D11} IgG1-Fc. (B) Schematic of IgG-Fc tag ectodomain of DR5 and UL141^{D11} (C) After expression in CHO cells, reducing gel confirming the size of UL141^{D11} and control molecules. (D) The binding kinetics of immobilized biotinylated rDR5 against UL141 and UL144 were measured using BLI. UL141 K_{D} (Binding affinity) against DR5 was 3.75 nM.

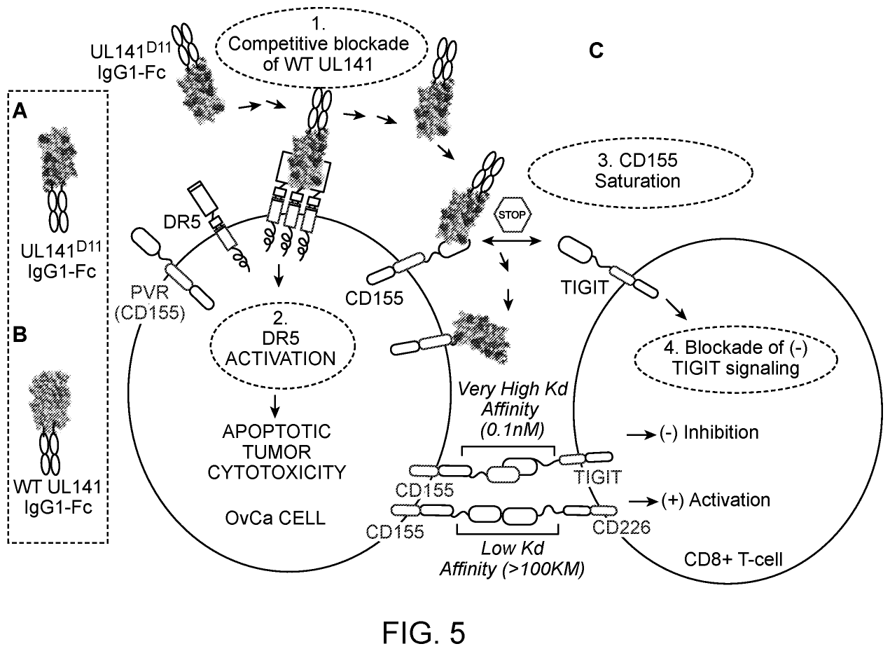

FIG. 5 provides engineered UL141 variant protein is a multi-prong effector molecule to target OvCa tumors. (A-B) Schematic of newly engineered UL141D11 IgG1-Fc and WT UL141 molecules. Blue sphere in (A) represents affinity matured DR5 activating mutations. (C). Externally added high affinity UL141D11 could not only (1) competitively interferes with viral hCMV UL141 (in infected patients) but also instigates (2) DR5 activation to activate tumor cytotoxicity in OvCa cells. Further, in tumor-immune interactions, tumor enriched CD155 could either activate T and NK cells via engaging CD226 (DNAM-1) or inhibit T and NK cell signaling via interacting with TIGIT. Since CD155-TIGIT interactions are super high affinity (<0.1 nM) compared to CD155-CD226 (~100 nM), immune inhibitory TIGIT activation by CD155+ tumor cells in preferred. The latter makes CD155-TIGIT a key immune-checkpoint molecule. Preliminary data supports the blockade of CD155-TIGIT interactions and signaling (3) by engineered UL141^{D11} variant.

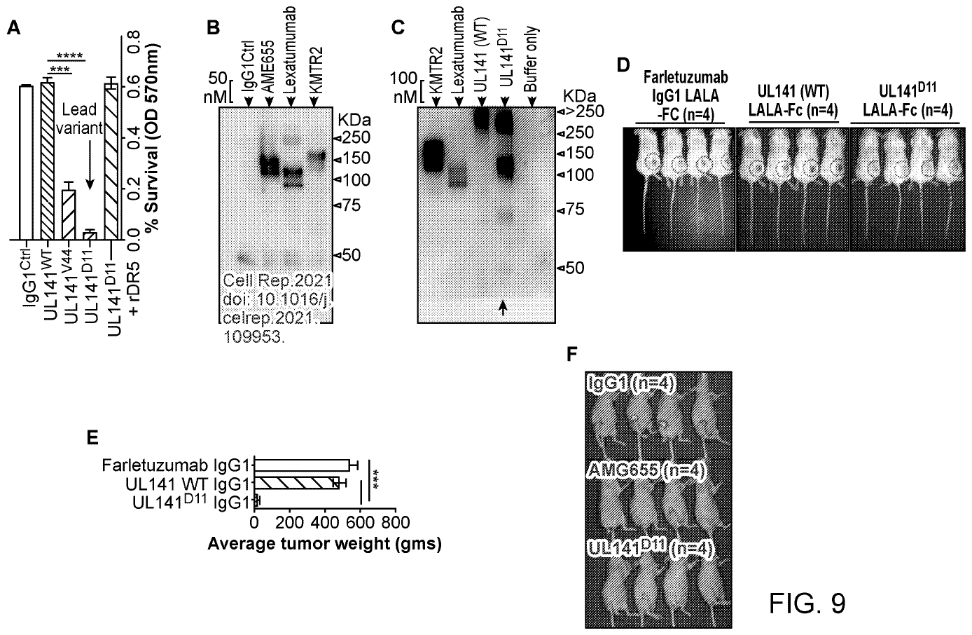

To test direct extracellular in vitro DR5 engagement by UL141^{D11}, expressed and purified were IgG4-Fc-conjugated extracellular fragment of recombinant DR5 (r-DR5) with IgG1-Fc variant: UL141^{D11} (FIG. 8c). Next, binding affinities were analyzed using ELISA (data not shown) and ForteBio Octet HTX (FIG. 8c). A very high affinity (~3.7 nM) interaction of UL141^{D11} with DR5 was observed. Preliminary UL141^{D11} showed the most increased cytotoxic activity against OvCa cells, comparable to clinical DR5 antibodies (FIG. 9a). Furthermore, in a cell clustering assay, unlike WT viral UL141, re-engineered UL141^{D11} showed DR5 clustering comparable to clinical DR5 antibodies (FIG. 9b-c). WT UL141 only generated high aggregates without intermediate ~150 KDa trimer (FIG. 9c). Since WT UL141 has no cytotoxic activity, the results support potential non-functional higher order aggregates by it. When tested in vivo, UL141^{D11} completely blocked the OVCAR-3 tumor growth (FIG. 9d-e). Importantly UL141^{D11} and clinical farletuzumab were engineered with ADCC inactivating LALA mutation in IgG1-Fc. Hence the inhibition of tumor growth by UL141^{D11} is due to DR5 mediated cytotoxicity (FIG. 9d-e). Similar efficacy results were seen with TNBC tumors (FIG. 9f).

FIG. 9 provides lead UL141 variant (UL141^{D11}) is highly cytotoxic to Ovarian and other DR5⁺ tumors. (A) The lead UL141^{D11} variant is an efficient cell-death activator while WT hCMV UL141 is totally ineffective. Importantly pre-neutralization of UL141^{D11} with recombinant-DR5 (rDR5) eliminates its activity completely. (B-C) The lead UL141^{D11} clusters DR5 similar or better than clinical antibodies (Lane 1, 2 vs. 4 in C) however WT UL141 only generated inactive higher aggregates (Lane 3 vs. 4 in C). (D-E) When tested against SQ grafted OVCAR-3 tumors on nude animals, UL141^{D11} was as an effective anti-tumor agent independent of ADCC function, while WT UL141 had no cytotoxic activity. (F) Similar in vivo efficacy results were seen with other DR5 positive tumors. UL141^{D11} was as effective as clinical DR5 antibody AMG655. It must be noted, unlike AMG655, UL141^{D11} also has an essential added T-cell activating function.

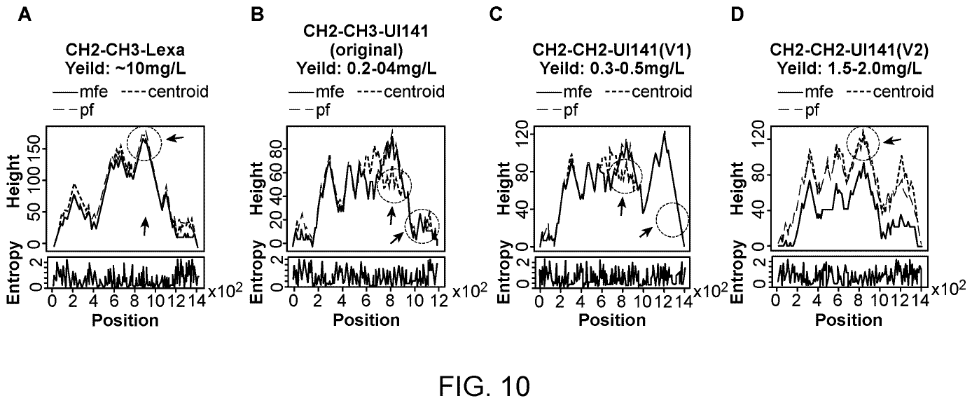

So far, the yield of UL141 variants has been considerably low compared to clinical DR5 agonist antibodies such as lexatumumab (FIG. 10a vs. b). Using codon optimization and the RNA fold program, over 20 different UL141 variants were tested, and their centroid prediction co-relation with expression in CHO cells. Strikingly, UL141 yield was improved using a CHO expression system up to 7-8 fold (FIG. 10d).

FIG. 10 provides improving UL141^{D11} yield in CHO expression system. Sequence of (A) Lexa, (B) UL141 original, (C) UL141^{D11} (V1) and (D) UL141^{D11} (V2) were analyzed using RNA fold webserver rna.tbi.univie.ac.at/cgi-bin/RNAWebSuite/RNAfold.cgi. The top part represents mountain plots, while the bottom shows free energy of respective RNA structures. In (C) UL141^{D11} (V1), is provided the codon optimized sequence around 0-20 height of mount plot (see bottom circle in B vs C) while in (D) UL141^{D11} (V2), is provided additionally optimized RNA sequence around 80-100 height (top circle in C vs D) of mountain plot to improve centroid plot similar to high yielding lexatumumab molecule (A). Significant improvement in yield was observed.

### Example 2

### CD155 Binding and Activation

In addition to DR5 targeting, one other study in 2005 described UL141's ability to sequester NK-cell and T-cell ligand CD155 (also known as Poliovirus Receptor, PVR) again in the endoplasmic reticulum (Smazynski et al., 2020, Gynecologic Oncology. 158(1):167-77). In the past decade, similar to DR5, CD155 has emerged as a key therapeutic target for OvCa and is commonly expressed on malignant epithelium in HGSOC, limiting T-cell infiltration in tumors (Smazynski et al.; Ozmadenci et al., 2022, P Natl Acad Sci USA. 119(17):1073). It was not until 2009 that NK and T-cell inhibiting and CD155 engaging receptor named TIGIT (T cell immunoreceptor with Ig and ITIM domains) was discovered (Yu et al., 2009, Nat Immunol. 10(1):48-57). The high-affinity CD155-TIGIT interactions in tumors inhibit tumor infiltered leukocytes (TILs) such as CD8+ T-cell activation in the microenvironment (Johnston et al., 2014, Cancer Cell. 26(6):923-37). The latter makes CD155-TIGIT a high-value immune checkpoint target similar to PD1-PD-L1. As a result, multiple anti-TIGIT antibodies are in clinical trials. It must be noted that all clinical anti-TIGIT antibodies are only designed to block CD155-TIGIT interactions. Hence if tested, they are expected to be limitedly effective against OvCa, similar to PD-L1 antibodies. Therefore, in the endless loop of cancer immunotherapy, automated focus on antibodies or strategies solely blocking checkpoint interactions limits the novel ideas of multifaceted alternate breakthrough targeting in ovarian and the solid cancer field. Importantly, due to its dual DR5 activating and CD155 sequestering mechanism, the engineered hCMV bug-derived UL141^{D11} has an inherent solution to overcome the clinical failures of DR5 and TIGIT antibodies despite over billion dollars spent on these clinical trials.

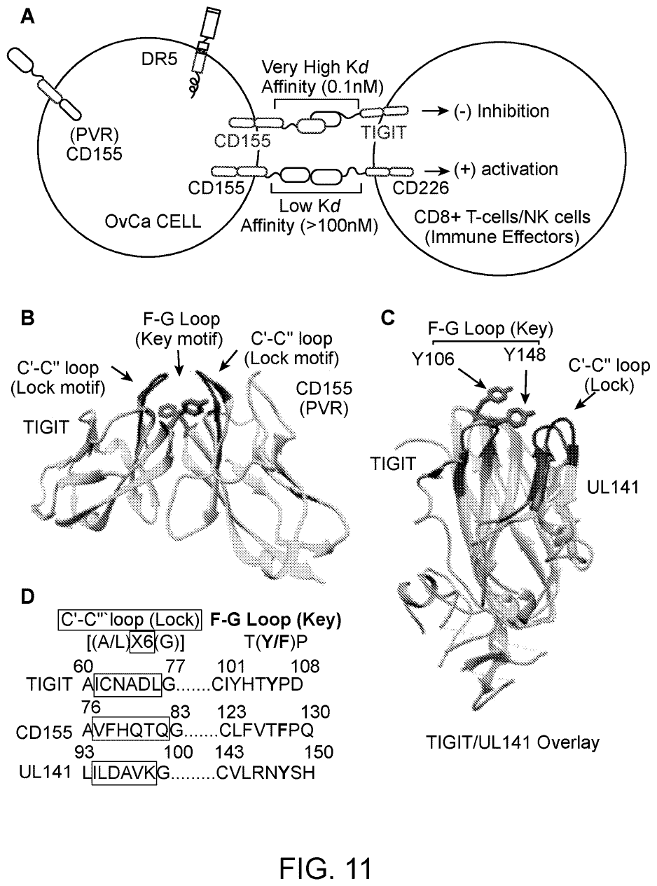

CD155, TIGIT, CD226, and UL141 all contain Ig-like domains with two unique and highly conserved sequences: “lock” A/L(X₆G) and “key” (T(F/Y)P) motifs (FIG. 11b-d) in the first immunoglobulin variable (IgV) domain (Zhang et al., 2004, Science 305(5681):251-4). When we screened for proteins with a domain structure similar to UL141 in the DALI server (Holm et al., 2010, Nucleic Acids Res. 2010; 38(Web Server issue):W545-9; Holm L., 2022, Nucleic Acids Res. Epub 2022/05/25). one of the top hits was CD155 binding partner, TIGIT (Z:7.4, rmsd:3.3).

78. Holm et al., 2010, Nucleic Acids Res. 2010; 38(Web Server issue):W545-9. Epub 2010/05/12. doi: 10.1093/nar/gkq366. PubMed PMID: 20457744; PMCID: PMC2896194.

79. Holm L. Dali server: structural unification of protein families. Nucleic Acids Res. 2022. Epub 2022/05/25. doi: 10.1093/nar/gkac387. PubMed PMID: 35610055.

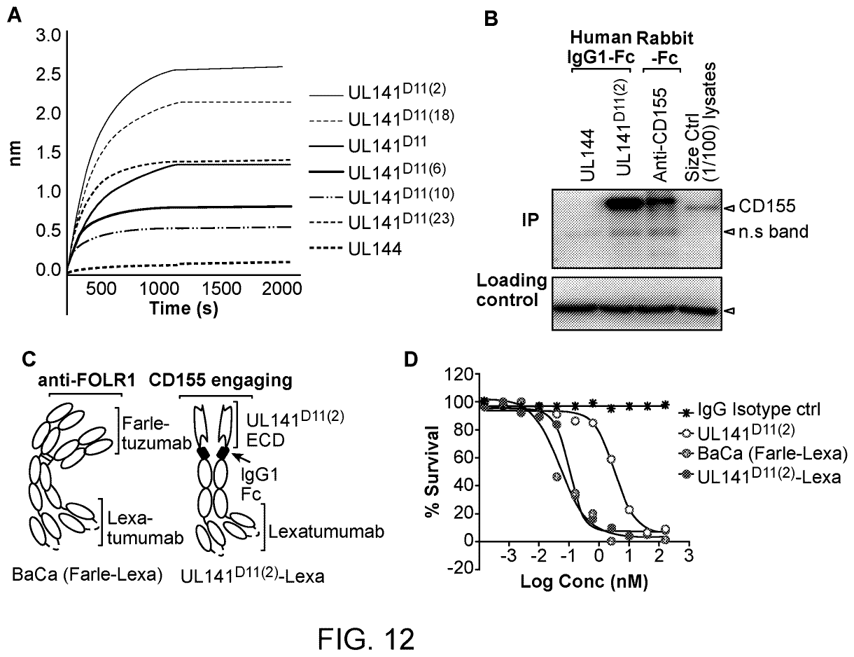

Next, TIGIT and UL141 were analyzed in structure comparison using the UCSF chimera overlay analysis function, which indicated a highly similar structural reorganization of TIGIT and UL141 “lock” and “key” motif with observed sequence conservation in them (FIG. 11c-d). Thus, to selectively enhance UL141^{D11} mediated CD155 saturation, UL141^{D11}'s lock motif region was further affinity matured for super high affinity (<0.001 nM) binding to CD155. The lead UL141^{D11(2)} showed high-affinity binding to CD155 in ForteBio Octet BLI assay (FIG. 12a) and immunoprecipitation assays using OVCAR-3 cells (FIG. 12b). Furthermore, when UL141^{D11(2)} was engineered with an anti-DR5 antibody lexatumumab into a bispecific molecule, it gained (IC₅₀>100-fold) enhanced cell killing activity (FIG. 12c-d). The latter further supports in vivo UL141^{D11(2)} binding against CD155.

FIG. 11 provides UL141 mirror TIGIT binding interface with CD155. (A) Tumor enriched CD155, a member of nectin family pairs with CD226 with low affinity to generate NK and T-cell activating immunological to promote cytokine secretion and anti-tumor response. On the contrary, the highly dominant and high affinity pairing of CD155 with TIGIT results in inhibition of NK/T-cell activation and proliferation, making CD155-TIGIT as a key immune checkpoint. (B) The immunoglobulin variable (IgV) domain of CD155 (cyan) and TIGIT (green) contains conserved lock and key motifs in C′-C″ and F-G loops of beta turns, to generate high affinity interactions. (C) UL141 (Gold) and TIGIT (green) share highly similar structures (including lock and key motifs) when overlaid together. The latter suggests similar binding pattern of TIGIT and UL141 against CD155 (D) The sequences of highly conserved double-lock-and-key motif [A/L(X6G)], and [T(F/Y)P] is shown for TIGIT, CD155 and UL141. Large hydrophobic residues either “Y” or “F” forms the core “key” motif to engage “lock” for high affinity binding. The latter supports high affinity TIGIT-CD155 interactions as compared to CD155-CD226.

FIG. 12 provides UL141 variant (UL141^{D11(2)}) binds CD155 with high affinity. (A) The binding kinetics of immobilized biotinylated CD155 against indicated UL141 variants were measured using BLL. (B) Immunoprecipitation of CD155 (from OVCAR-3 cells) using UL141^{D11(2)} IgG1 Fc. UL144 is negative control and IP with commercial CD155 antibody is positive control. (C) Schematic showing anti-FOLR1 and anti-DR5 lexatumumab as a bispecific molecule (BaCa, left) and UL141^{D11(2)} and lexatumumab containing UL141^{D11(2)}-Lexa bispecific molecule (right). (D) Cell killing assays of UL141^{D11(2)} alone, BaCa and UL141^{D11(2)}-Lexa molecule using OVCAR-3 cells.

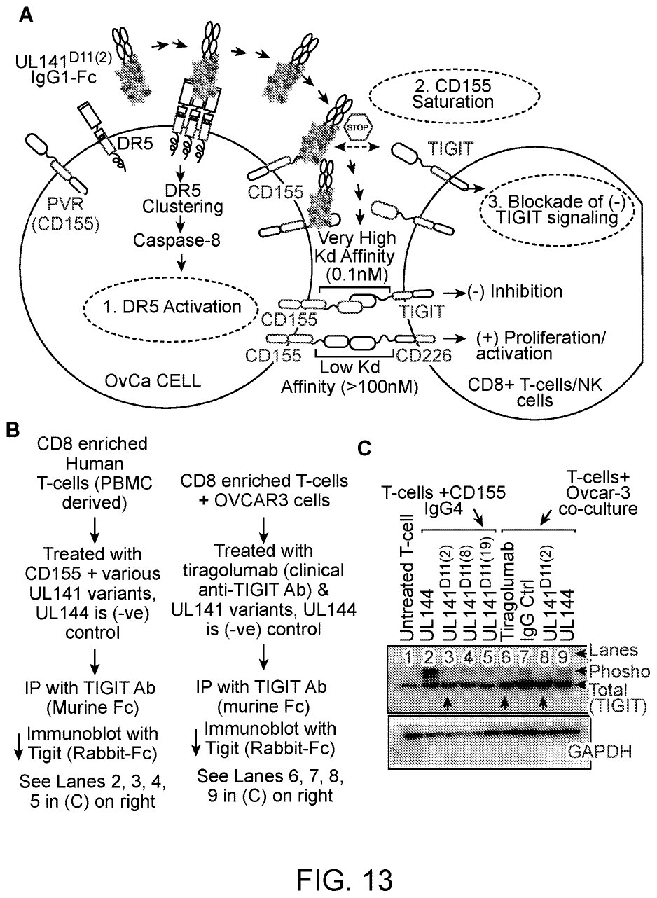

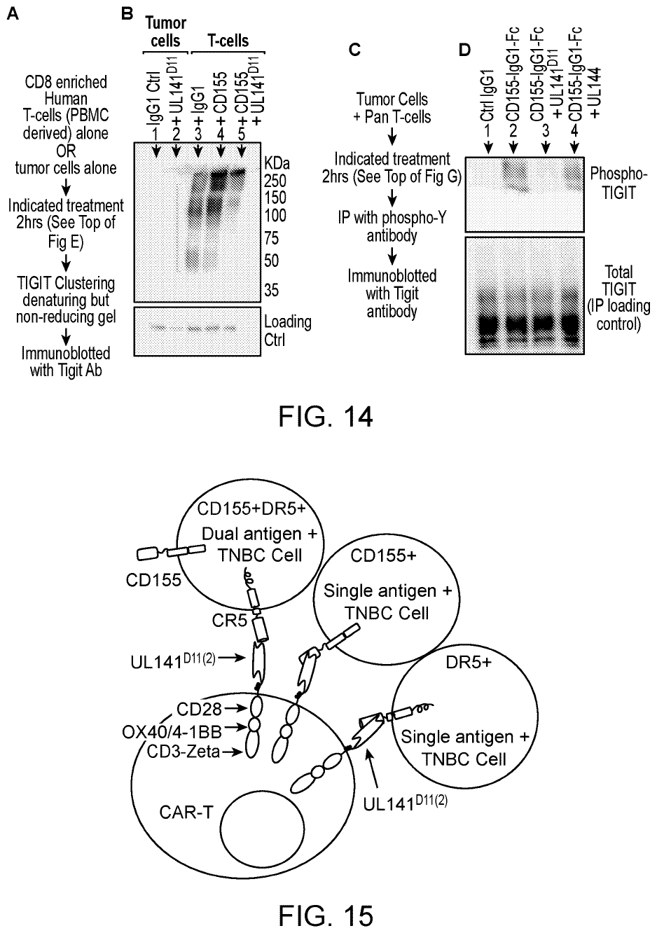

Considering the data described in Example 1 and the combination of data shown in FIGS. 11 and 12, it is evident that UL141^{D11(2)} can saturate CD155 to inhibit immune suppressive TIGIT signaling while maintaining its original DR5 activating function (FIG. 13a). In studies using UL141^{D11(2)} next tested was TIGIT phosphorylation of its ITIM domain at tyrosine Y225 or Y231 upon adding either CD155-His alone or in the presence of UL141^{D11(2)} LL141^{D11(2)} inhibited T-cell negative regulatory TIGIT phosphorylation (FIG. 13b-c). Next, tested was TIGIT clustering: an important biochemical event that precedes immune-inhibitory signaling upon pairing with CD155. For these studies, CD155+ve tumor cells were co-cultured with T-cells (isolated from huPBMC) in the presence of UL141^{D11(2)} and other control molecules. As expected, not only was observed blockade of TIGIT clustering by CD155 (FIG. 14a-b) but also the complete inhibition of its phosphorylation at Y225 and Y231 by UL141^{D11(2)} variant but not by control non-DR5 and non-CD155 engaging UL144 (FIG. 14c-d).

FIG. 13 provides UL141^{D14(2)} inhibits CD155-TIGIT pairing and T-cell inhibitory signaling. (A) The reverse engineered UL141 variant, UL141^{D14(2)} is a versatile multi-faceted molecule. (1) UL141^{D14(2)} directly activates DR5 to instigate apoptotic cell death. (2) UL141^{D14(2)} saturates CD155 at tumor-T-cell interface. (3) The latter inhibits the high affinity CD155-TIGIT pairing as well as T-cell immune suppressive signaling. (B) CD8+ T-cells were either treated with CD155 IgG1 or CD155+ OVCAR-3 cells in presence of UL141^{D14(2)} and other controls including +ve control anti-TIGIT antibody followed by immunoprecipitation with TIGIT antibody to detect total and phosphorylated TIGIT. (C). UL141^{D14(2)} selectively inhibited TIGIT phosphorylation similar to clinical TIGIT antibody, tiragolumab.

FIG. 14 provides UL141^{D14(2)} inhibits CD155-TIGIT clustering, pairing and T-cell inhibitory signaling. (A) TNBC tumor cells and T-cells were cocultured and treated as described in schematic. (B) Clustering profile of TIGIT in non-reducing but denaturing gel. Compare lane 3 and 4 with UL141^{D14(2)} treated lane 5. (C) Schematic showing experimental detail of tumor cells and T-cells coculture to analyze TIGIT tyrosine phosphorylation. (D) UL141^{D14(2)} inhibits TIGIT phosphorylation in the tumor cells and T-cells coculture experiment. Compare lane 2 vs 3. Bottom blot shows total TIGIT levels prior to phosphor tyrosine immunoprecipitation.

### Example 3

### CAR-T and CAR-NK Therapy

Many antibodies which bind, target and function in IgG1 format as Fab, also work as CAR-T and CAR-NK cells if infused with T-cell activating construct cytotoxic. Also provided UL141^{D11} and UL141^{D11(2)} and other variants expressing alternate CAR-NK and CAR-T cells (FIG. 15).

FIG. 15 provides UL141^{D11(2)} CAR-T (or CAR-NK) as an alternate strategy. In heterogenous OvCa tumors, cells expressing either single antigen (CD155 alone or DR5 alone) or dual antigen will be targeted by CAR-T cells stably expressing UL141^{D11(2} on the cell surface.

### Example 4

### Cellular Localization of UL141

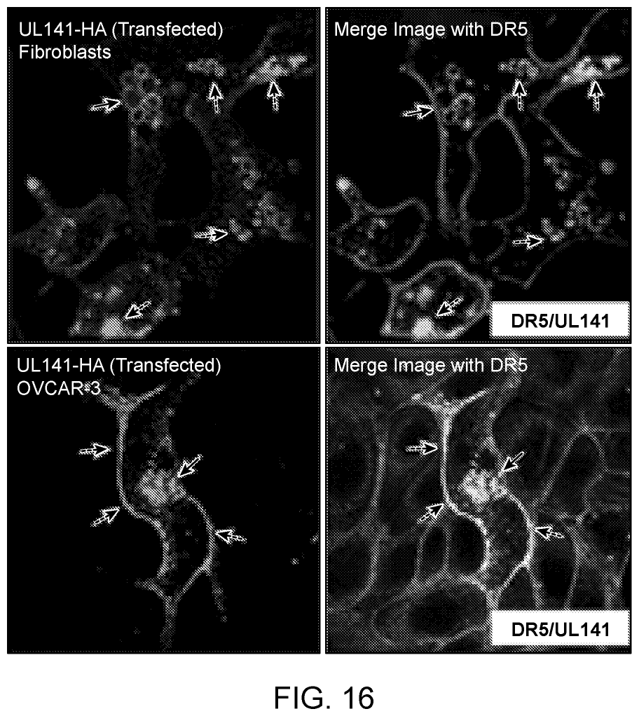

UL141-HA was transfected in human fibroblasts (FIG. 16, top) and OVCAR-3 cells (FIG. 16, bottom). Cells were stained for DR5 (red) and UL141-HA (green). UL141 showed a vesicular pattern and ER enriched pattern in fibroblasts, while it co-localized with DR5 on membrane (yellow co-localized signal, yellow arrows) in tumor cells (FIG. 16, right). These results show that transfected hCMV UL141 consistently localizes at the cell surface of tumor cells versus fibroblasts.

### Example 5

Binding Studies with CD155 and CD226

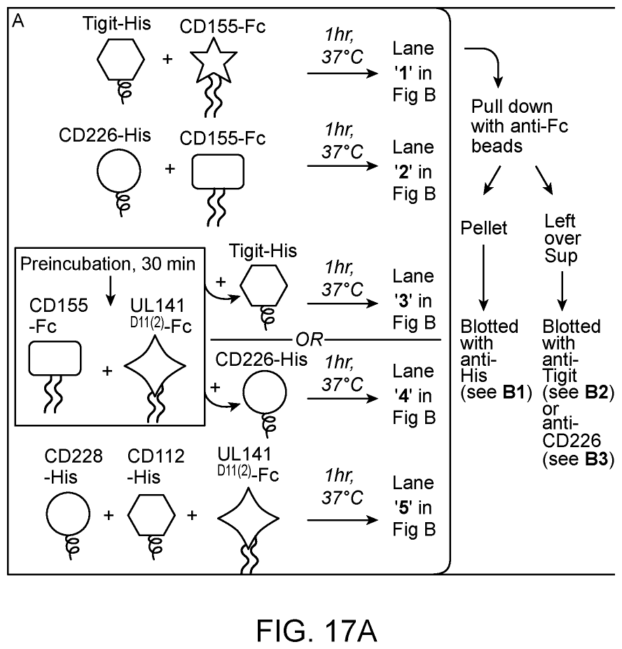

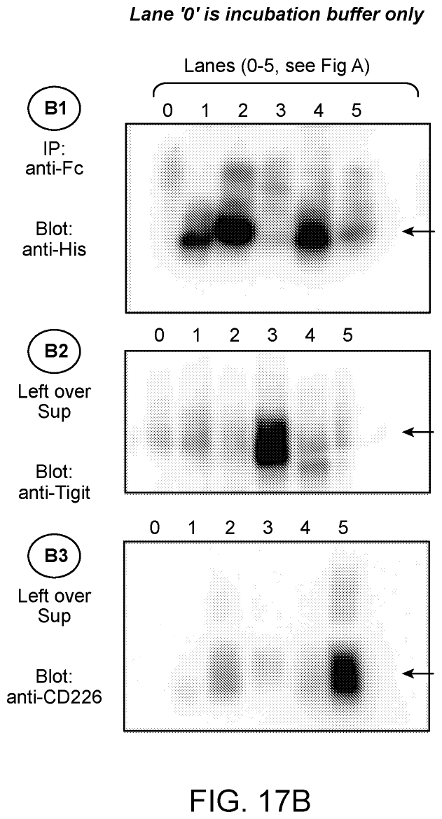

A mixing experiment was performed with various indicated His of Fc-tagged proteins followed by pulldown with anti-Fc magnetic beads as shown in FIG. 17A. The precipitates from the pull down (B1) and leftover supernatants (B2, B3) were run on SDS-PAGE and immunoblotted with anti-His (B1: IP precipitates), anti-TIGIT (B2 leftover supernatant) and anti-CD226 (B3 leftover supernatant) antibodies as shown in FIG. 17B. TIGIT was not pulled down by CD155-UL141^{D11(2)} complex (Compare Lane 3: B1 vs. B2), while CD226 was pulled down by the same (Compare Lane 4: B1 vs. B2). These results show that UL141^{D11(2)} binding to CD155 does not appear to interfere with the ability of CD155 to engage CD226.

### Example 6

### Binding Studies and Cell Killing Assay

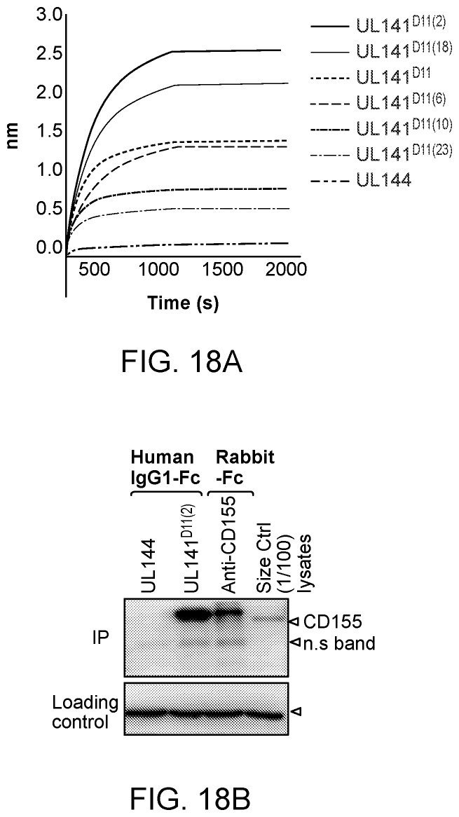

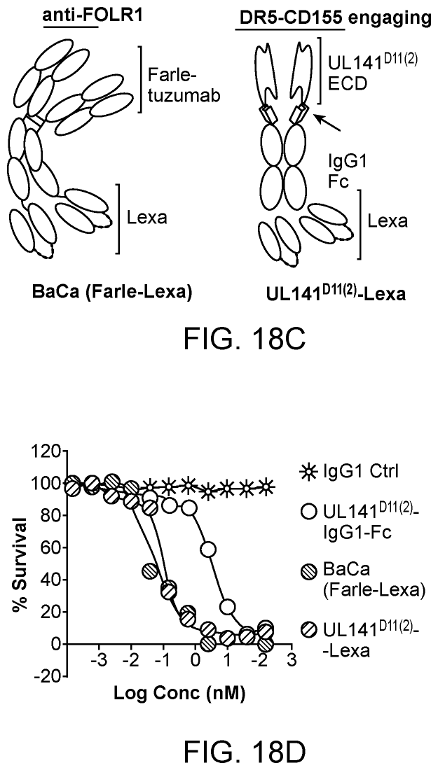

The binding kinetics of immobilized, biotinylated CD155 against indicated UL141 variants were measured using BLI as shown in FIG. 18A. Results for immunoprecipitation of CD155 (OVCAR-3 cells) using UL141^{D11(2)}IgG1 Fc UL144 is −ve control, IP with commercial antibody is +ve control as shown in FIG. 18B. FIG. 18C shows a schematic of anti-FOLR1 and anti-DR5 lexatumomab as a bispecific molecule (BaCa, left) and UL141^{D11(2)} and lexatumumab containing UL141^{D11(2)}-Lexa as a bispecific molecule (right). Results for cell killing assays of UL141^{D11(2)} alone, BaCa and UL141^{D11(2)}-Lexa using OVCAR-3 cells are shown in FIG. 18D. These results demonstrate that UL141^{D11(2)} binds CD155 with high affinity.

### Example 7

### CD155:TIGIT Pairing Inhibition and T-Cell Inhibitor Signaling

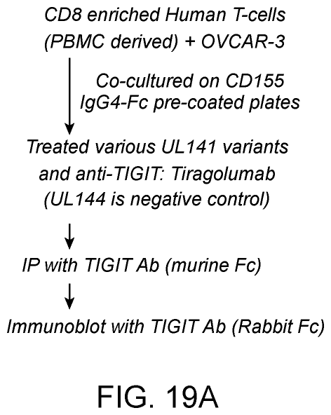

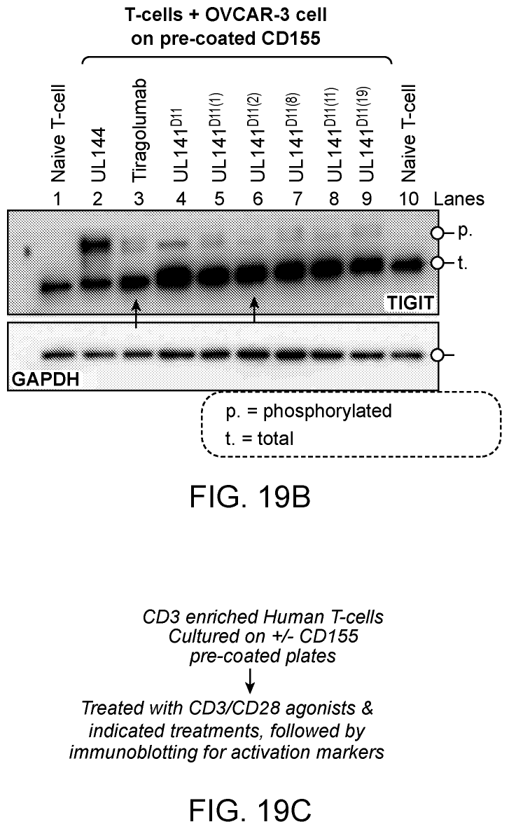

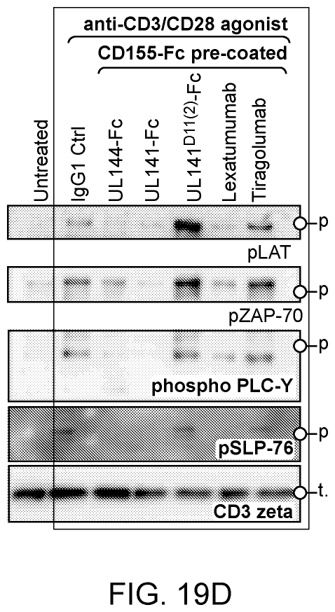

CD8⁺ T-cells and OCVAR-3 cells (2:1) were plated on CD155-IgG4Fc precoated plates followed by various indicated treatments after 12 hours as indicated in FIG. 19A. Immunoblotting data from this procedure is shown in FIG. 18B. CD3-enriched human PBMC were cultured on CD155-IgG4Fc-coated plates. The cells were treated with T-cell activation mix (CD3/CD28) in the presence of various indicated UL141 variants, various controls (UL144, FOLR1, DR5) and clinical anti-TIGIT antibody tiragolumab as shown in FIG. 19C. Results for immunoblotting of the lysates after 2 hours are shown for indicated T-cell activation markers (FIG. 19D). These results show that UL141^{D11(2)} inhibits CD155-TIGIT pairing and T-cell inhibitory signaling.

### Example 8

SEQUENCES

| Name | UL141 Sequence | Fusion Sequence | Name |
|---|---|---|---|
| UL141_1 | SFPFATADIAEKMWAENYETTS | SFPFATADIAEKMWAENYETTSP | Fc fusion |
| 30-162 | PAPVLVAEGEQVTIPCTVMTHS | APVLVAEGEQVTIPCTVMTHSWP | SEQ ID NO: 34 |
| SEQ ID | WPMVSIRARFCRSHDGSDELIL | MVSIRARFCRSHDGSDELILDAV | |
| NO: 2 | DAVKGHRLMNGLQYRLPYATWN | KGHRLMNGLQYRLPYATWNFSQL | |
| | FSQLHLGQIFSLTFNVSTDTAG | HLGQIFSLTFNVSTDTAGMYECV | |
| | MYECVLRNYSHGLIMQRFVILT | LRNYSHGLIMQRFVILTQLESGG | |
| | QLE | GSGGSESKYGPPCPPCPAPEFLG | |
| | | GPSVFLFPPKPKDTLMISRTPEV | |
| | | TCVVVDVSQEDPEVQFNWYVDGV | |
| | | EVHNAKTKPREEQFNSTYRVVSV | |
| | | LTVLHQDWLNGKEYKCKVSNKGL | |
| | | PSSIEKTISKAKGQPREPQVYTL | |
| | | PPSQEEMTKNQVSLTCLVKGFYP | |
| | | SDIAVEWESNGQPENNYKTTPPV | |
| | | LDSDGSFFLYSKLTVDKSRWQEG | |
| | | NVFSCSVMHEALHNHYTQKSLSL | |
| | | SLG | |
| UL141_2 | QLETLSRPDEPCCTPALGRYSL | QLETLSRPDEPCCTPALGRYSLG | Fc fusion |
| 162-279 | GDQIWSPTPWRLRNHDCGMYRG | DQIWSPTPWRLRNHDCGMYRGFQ | SEQ ID NO: 35 |
| SEQ ID | FQRNYFYIGRADAEDCWKPACP | RNYFYIGRADAEDCWKPACPDEE | |
| NO: 3 | DEEPDRCWTVIQRYRLPGDCYR | PDRCWTVIQRYRLPGDCYRSQPH | |
| | SQPHPPKFLPVTPAPPADIDTG | PPKFLPVTPAPPADIDTGMSPWA | |
| | MSPWATR | TRSGGGSGGSEPKSCDKTHTCPP | |
| | | CPAPELLGGPSVFLFPPKPKDTL | |
| | | MISRTPEVTCVVVDVEHEDPEVK | |
| | | FNWYVDGVEVHNAKTKPREEQYN | |
| | | STYRVVSVLTVLHQDWLNGKEYK | |
| | | CKVSNKALPAPIEKTISKAKGQP | |
| | | REPQVYTLPPSREEMTKNQVSLT | |
| | | CLVKGFYPSDIAVEWESNGQPEN | |
| | | NYKTTPPVLDSDGSFFLYSKLTV | |
| | | DKSRWQQGNVFSCSVMHEALHNH | |
| | | YTQKSLSLSLGK | |
| UL141_W | SFPFATADIAEKMWAENYETTS | SFPFATADIAEKMWAENYETTSP | Fc fusion |
| 30-172 | PAPVLVAEGEQVTIPCTVMTHS | APVLVAEGEQVTIPCTVMTHSWP | SEQ ID NO: 36 |
| SEQ ID | WPMVSIRARFCRSHDGSDELIL | MVSIRARFCRSHDGSDELILDAV | |
| NO : 4 | DAVKGHRLMNGLQYRLPYATWN | KGHRLMNGLQYRLPYATWNFSQL | |
| | FSQLHLGQIFSLTENVSTDTAG | HLGQIFSLTFNVSTDTAGMYECV | |
| | MYECVLRNYSHGLIMQRFVILT | LRNYSHGLIMQRFVILTQLESRP | |
| | QLESRPDEPCCTP | DEPCCTPSGGGSGGSESKYGPPC | |
| | | PPCPAPEFLGGPSVFLFPPKPKD | |
| | | TLMISRTPEVTCVVVDVSQEDPE | |
| | | VQFNWYVDGVEVHNAKTKPREEQ | |
| | | FNSTYRVVSVLTVLHQDWLNGKE | |
| | | YKCKVSNKGLPSSIEKTISKAKG | |
| | | QPREPQVYTLPPSQEEMTKNQVS | |
| | | LTCLVKGFYPSDIAVEWESNGQP | |
| | | ENNYKTTPPVLDSDGSFFLYSKL | |
| | | TVDKSRWQEGNVFSCSVMHEALH | |
| | | NHYTQKSLSLSLG | |
| UL141_4 | SFPFATADIAEKMWAENYETTS | SFPFATADIAEKMWAENYETTSP | Fc fusion |
| 30-182 | PAPVLVAEGEQVTIPCTVMTHS | APVLVAEGEQVTIPCTVMTHSWP | SEQ ID NO: 37 |
| SEQ ID | WPMVSIRARFCRSHDGSDELIL | MVSIRARFCRSHDGSDELILDAV | |
| NO : 5 | DAVKGHRLMNGLQYRLPYATWN | KGHRLMNGLQYRLPYATWNFSQL | |
| | FSQLHLGQIFSLTFNVSTDTAG | HLGQIFSLTFNVSTDTAGMYECV | |
| | MYECVLRNYSHGLIMQRFVILT | LRNYSHGLIMQRFVILTQLESRP | |
| | QLESRPDEPCCTPALGRYSLGD | DEPCCTPALGRYSLGDQSGGGSG | |
| | Q | GSESKYGPPCPPCPAPEFLGGPS | |
| | | VFLFPPKPKDTLMISRTPEVTCV | |
| | | VVDVSQEDPEVQFNWYVDGVEVH | |
| | | NAKTKPREEQFNSTYRVVSVLTV | |
| | | LHQDWLNGKEYKCKVSNKGLPSS | |
| | | IEKTISKAKGQPREPQVYTLPPS | |
| | | QEEMTKNQVSLTCLVKGFYPSDI | |
| | | AVEWESNGQPENNYKTTPPVLDS | |
| | | DGSFFLYSKLTVDKSRWQEGNVF | |
| | | SCSVMHEALHNHYTQKSLSLSLG | |
| UL141_5 | SFPFATADIAEKMWAENYETTS | SFPFATADIAEKMWAENYETTSP | Fc fusion |
| 30-192 | PAPVLVAEGEQVTIPCTVMTHS | APVLVAEGEQVTIPCTVMTHSWP | SEQ ID NO: 38 |
| SEQ ID | WPMVSIRARFCRSHDGSDELIL | MVSIRARFCRSHDGSDELILDAV | |
| NO : 6 | DAVKGHRLMNGLQYRLPYATWN | KGHRLMNGLQYRLPYATWNFSQL | |
| | FSQLHLGQIFSLTFNVSTDTAG | HLGQIFSLTFNVSTDTAGMYECV | |
| | MYECVLRNYSHGLIMQRFVILT | LRNYSHGLIMQRFVILTQLESRP | |
| | QLESRPDEPCCTPALGRYSLGD | DEPCCTPALGRYSLGDQSGGGSG | |
| | Q | GSESKYGPPCPPCPAPEFLGGPS | |
| | | VFLFPPKPKDTLMISRTPEVTCV | |
| | | VVDVSQEDPEVQFNWYVDGVEVH | |
| | | NAKTKPREEQFNSTYRVVSVLTV | |
| | | LHQDWLNGKEYKCKVSNKGLPSS | |
| | | IEKTISKAKGQPREPQVYTLPPS | |
| | | QEEMTKNQVSLTCLVKGFYPSDI | |
| | | AVEWESNGQPENNYKTTPPVLDS | |
| | | DGSFFLYSKLTVDKSRWQEGNVE | |
| | | SCSVMHEALHNHYTQKSLSLSLG | |
| UL141_6 | SFPFATADIAEKMWAENYETTS | SFPFATADIAEKMWAENYETTSP | Fc fusion |
| 30-202 | PAPVLVAEGEQVTIPCTVMTHS | APVLVAEGEQVTIPCTVMTHSWP | SEQ ID NO: 39 |
| SEQ ID | WPMVSIRARFCRSHDGSDELIL | MVSIRARFCRSHDGSDELILDAV | |
| NO: 7 | DAVKGHRLMNGLQYRLPYATWN | KGHRLMNGLQYRLPYATWNFSQL | |
| | FSQLHLGQIFSLTFNVSTDTAG | HLGQIFSLTFNVSTDTAGMYECV | |
| | MYECVLRNYSHGLIMQRFVILT | LRNYSHGLIMQRFVILTQLESRP | |
| | QLESRPDEPCCTPALGRYSLGD | DEPCCTPALGRYSLGDQIWSPTP | |
| | QIWSPTPWRLR | WRLRSGGGSGGSESKYGPPCPPC | |
| | | PAPEFLGGPSVFLFPPKPKDTLM | |
| | | ISRTPEVTCVVVDVSQEDPEVQF | |
| | | NWYVDGVEVHNAKTKPREEQENS | |
| | | TYRVVSVLTVLHQDWLNGKEYKC | |
| | | KVSNKGLPSSIEKTISKAKGQPR | |
| | | EPQVYTLPPSQEEMTKNQVSLTC | |
| | | LVKGFYPSDIAVEWESNGQPENN | |
| | | YKTTPPVLDSDGSFFLYSKLTVD | |
| | | KSRWQEGNVFSCSVMHEALHNHY | |
| | | TQKSLSLSLG | |
| UL141_7 | SFPFATADIAEKMWAENYETTS | SFPFATADIAEKMWAENYETTSP | Fc fusion |
| 30-212 | PAPVLVAEGEQVTIPCTVMTHS | APVLVAEGEQVTIPCTVMTHSWP | SEQ ID NO: 40 |
| SEQ ID | WPMVSIRARFCRSHDGSDELIL | MVSIRARFCRSHDGSDELILDAV | |
| NO : 8 | DAVKGHRLMNGLQYRLPYATWN | KGHRLMNGLQYRLPYATWNFSQL | |
| | FSQLHLGQIFSLTFNVSTDTAG | HLGQIFSLTFNVSTDTAGMYECV | |
| | MYECVLRNYSHGLIMQRFVILT | LRNYSHGLIMQRFVILTQLESRP | |
| | QLESRPDEPCCTPALGRYSLGD | DEPCCTPALGRYSLGDQIWSPTP | |
| | QIWSPTPWRLRNHDCGMYRGF | WRLRNHDCGMYRGFSGGGSGGSE | |
| | | SKYGPPCPPCPAPEFLGGPSVFL | |
| | | FPPKPKDTLMISRTPEVTCVVVD | |
| | | VSQEDPEVQFNWYVDGVEVHNAK | |
| | | TKPREEQFNSTYRVVSVLTVLHQ | |
| | | DWLNGKEYKCKVSNKGLPSSIEK | |
| | | TISKAKGQPREPQVYTLPPSQEE | |
| | | MTKNQVSLTCLVKGFYPSDIAVE | |
| | | WESNGQPENNYKTTPPVLDSDGS | |
| | | FFLYSKLTVDKSRWQEGNVESCS | |
| | | VMHEALHNHYTQKSLSLSLG | |
| UL141_8 | SFPFATADIAEKMWAENYETTS | SFPFATADIAEKMWAENYETTSP | Fc fusion |
| 30-222 | PAPVLVAEGEQVTIPCTVMTHS | APVLVAEGEQVTIPCTVMTHSWP | SEQ ID NO: 41 |
| SEQ ID | WPMVSIRARFCRSHDGSDELIL | MVSIRARFCRSHDGSDELILDAV | |
| NO : 9 | DAVKGHRLMNGLQYRLPYATWN | KGHRLMNGLQYRLPYATWNFSQL | |
| | FSQLHLGQIFSLTFNVSTDTAG | HLGQIFSLTFNVSTDTAGMYECV | |
| | MYECVLRNYSHGLIMQRFVILT | LRNYSHGLIMQRFVILTQLESRP | |
| | QLESRPDEPCCTPALGRYSLGD | DEPCCTPALGRYSLGDQIWSPTP | |
| | QIWSPTPWRLRNHDCGMYRGFQ | WRLRNHDCGMYRGFQRNYFYIGR | |
| | RNYFYIGRA | ASGGGSGGSESKYGPPCPPCPAP | |
| | | EFLGGPSVFLFPPKPKDTLMISR | |
| | | TPEVTCVVVDVSQEDPEVQFNWY | |
| | | VDGVEVHNAKTKPREEQFNSTYR | |
| | | VVSVLTVLHQDWLNGKEYKCKVS | |
| | | NKGLPSSIEKTISKAKGQPREPQ | |
| | | VYTLPPSQEEMTKNQVSLTCLVK | |
| | | GFYPSDIAVEWESNGQPENNYKT | |
| | | TPPVLDSDGSFFLYSKLTVDKSR | |
| | | WQEGNVFSCSVMHEALHNHYTQK | |
| | | SLSLSLG | |
| UL141_9 | SFPFATADIAEKMWAENYETTS | SFPFATADIAEKMWAENYETTSP | Fc fusion |
| 30-232 | PAPVLVAEGEQVTIPCTVMTHS | APVLVAEGEQVTIPCTVMTHSWP | SEQ ID NO: 42 |
| SEQ ID | WPMVSIRARFCRSHDGSDELIL | MVSIRARFCRSHDGSDELILDAV | |
| NO: 10 | DAVKGHRLMNGLQYRLPYATWN | KGHRLMNGLQYRLPYATWNFSQL | |
| | FSQLHLGQIFSLTFNVSTDTAG | HLGQIFSLTFNVSTDTAGMYECV | |
| | MYECVLRNYSHGLIMQRFVILT | LRNYSHGLIMQRFVILTQLETLS | |
| | QLETLSRPDEPCCTPALGRYSL | RPDEPCCTPALGRYSLGDQIWSP | |
| | GDQIWSPTPWRLRNHDCGMYRG | TPWRLRNHDCGMYRGFQRNYFYI | |
| | FQRNYFYIGRADAEDCWKPACP | GRADAEDCWKPACPSGGGSGGSE | |
| | | SKYGPPCPPCPAPEFLGGPSVFL | |
| | | FPPKPKDTLMISRTPEVTCVVVD | |
| | | VSQEDPEVQFNWYVDGVEVHNAK | |
| | | TKPREEQFNSTYRVVSVLTVLHQ | |
| | | DWLNGKEYKCKVSNKGLPSSIEK | |
| | | TISKAKGQPREPQVYTLPPSQEE | |
| | | MTKNQVSLTCLVKGFYPSDIAVE | |
| | | WESNGQPENNYKTTPPVLDSDGS | |
| | | FFLYSKLTVDKSRWQEGNVFSCS | |
| | | VMHEALHNHYTQKSLSLSLG | |
| UL141_10 | SFPFATADIAEKMWAENYETTS | SFPFATADIAEKMWAENYETTSP | Fc fusion |
| 30-217- | PAPVLVAEGEQVTIPCTVMTHS | APVLVAEGEQVTIPCTVMTHSWP | SEQ ID NO: 43 |
| 224-270 | WPMVSIRARFCRSHDGSDELIL | MVSIRARFCRSHDGSDELILDAV | |
| SEQ ID | DAVKGHRLMNGLQYRLPYATWN | KGHRLMNGLQYRLPYATWNFSQL | |
| NO: 11 | FSQLHLGQIFSLTFNVSTDTAG | HLGQIFSLTFNVSTDTAGMYECV | |
| | MYECVLRNYSHGLIMQRFVILT | LRNYSHGLIMQRFVILTLSSGGG | |
| | LSSGGGSPACPDEEPDRCWTVI | SPACPDEEPDRCWTVIQEYELPG | |
| | QEYELPGDCYRSQPHPPKFLPV | DCYRSQPHPPKFLPVTPAPPADI | |
| | TPAPPADIDTGMSPWATR | DTGMSPWATRSGGGSGGSESKYG | |
| | | PPCPPCPAPEFLGGPSVFLFPPK | |
| | | PKDTLMISRTPEVTCVVVDVSQE | |
| | | DPEVQFNWYVDGVEVHNAKTKPR | |
| | | EEQFNSTYRVVSVLTVLHQDWLN | |
| | | GKEYKCKVSNKGLPSSIEKTISK | |
| | | AKGQPREPQVYTLPPSQEEMTKN | |
| | | QVSLTCLVKGFYPSDIAVEWESN | |
| | | GQPENNYKTTPPVLDSDGSFFLY | |
| | | SKLTVDKSRWQEGNVFSCSVMHE | |
| | | ALHNHYTQKSLSLSLG | |
| UL141_11 | SFPFATADIAEKMWAENYETTS | SFPFATADIAEKMWAENYETTSP | Fc fusion |
| SEQ ID | PAPVLVAEGEQVTIPCTVMTHS | APVLVAEGEQVTIPCTVMTHSWP | SEQ ID NO: 44 |
| NO: 12 | WPMVSIRARFCRSHDGSDEAIL | MVSIRARFCRSHDGSDEAILNAD | |
| | NADLGHRLMNGLQYRLPYATWN | LGHRLMNGLQYRLPYATWNFSQL | |
| | FSQLHLGQIFSLTFNVSTDTAG | HLGQIFSLTFNVSTDTAGMYECV | |
| | MYECVLHTYPDGLIMQRFVILT | LHTYPDGLIMQRFVILTQLETLS | |
| | QLETLSRPDEPCCTPALGRYSL | RPDEPCCTPALGRYSLGDQIWSP | |
| | GDQIWSPTPWRLRNHDCGMYRG | TPWRLRNHDCGMYRGFQRNYFYI | |
| | FQRNYFYIGRADAEDCWKPACP | GRADAEDCWKPACPDEEPDRCWT | |
| | DEEPDRCWTVIQEYELPGDCYR | VIQEYELPGDCYRSQPHPPKFLP | |
| | SQPHPPKFLPVTPAPPADIDTG | VTPAPPADIDTGMSPWATRSGGG | |
| | MSPWATR | SGGSESKYGPPCPPCPAPEFLGG | |
| | | PSVFLFPPKPKDTLMISRTPEVT | |
| | | CVVVDVSQEDPEVQFNWYVDGVE | |
| | | VHNAKTKPREEQFNSTYRVVSVL | |
| | | TVLHQDWLNGKEYKCKVSNKGLP | |
| | | SSIEKTISKAKGQPREPQVYTLP | |
| | | PSQEEMTKNQVSLTCLVKGFYPS | |
| | | DIAVEWESNGQPENNYKTTPPVL | |
| | | DSDGSFFLYSKLTVDKSRWQEGN | |
| | | VFSCSVMHEALHNHYTQKSLSLS | |
| | | LG | |
| UL141_11 | SFPFATADIAEKMWAENYETTS | SFPFATADIAEKMWAENYETTSP | Fc fusion |
| (2) | WPMVSIRARFCRSHDGSDELIL | APVLVAEGEQVTIPCTVMTHSWP | SEQ ID NO: 45 |
| SEQ ID | NADLGHRLMNGLQYRLPFATWN | MVSIRARFCRSHDGSDELILNAD | |
| NO: 13 | FSQLHLGQIYSLTFNVSTDTAG | LGHRLMNGLQYRLPFATWNFSQL | |
| | MYECVLRNYPHGLIMQRFVILT | HLGQIYSLTFNVSTDTAGMYECV | |
| | QLETLSRPDEPCCTPALGRYSL | LRNYPHGLIMQRFVILTQLETLS | |
| | GDQIWSPTPWRLRNHDCGMYRG | RPDEPCCTPALGRYSLGDQIWSP | |
| | FQRNYFYIGRADAEDCWKPACP | TPWRLRNHDCGMYRGFQRNYFYI | |
| | DEEPDRCWTVIQEPELPGDCYR | GRADAEDCWKPACPDEEPDRCWT | |
| | SQPHPPKFLPVTPAPPADIDTG | VIQEPELPGDCYRSQPHPPKFLP | |
| | MSPWATR | VTPAPPADIDTGMSPWATRSGAA | |
| | | AGAAAESKYGPPCPPCPAPEFLG | |
| | | GPSVFLFPPKPKDTLMISRTPEV | |
| | | TCVVVDVSQEDPEVQFNWYVDGV | |
| | | EVHNAKTKPREEQFNSTYRVVSV | |
| | | LTVLHQDWLNGKEYKCKVSNKGL | |
| | | PSSIEKTISKAKGQPREPQVYTL | |
| | | PPSQEEMTKNQVSLTCLVKGFYP | |
| | | SDIAVEWESNGQPENNYKTTPPV | |
| | | LDSDGSFFLYSKLTVDKSRWQEG | |
| | | NVFSCSVMHEALHNHYTQKSLSL | |
| | | SLG | |
| UL141_11 | SFPFATADIAEKMWAENYETTS | SFPFATADIAEKMWAENYETTSP | Fc fusion |
| (2A) | PAPVLVAEGEQVTIPCTVMTHS | APVLVAEGEQVTIPCTVMTHSWP | SEQ ID NO: 46 |
| SEQ ID | WPMVSIRARFCRSHDGSDELIL | MVSIRARFCRSHDGSDELILNAD | |
| NO: 14 | NADLGHRLMNGLQYRLPFATWN | LGHRLMNGLQYRLPFATWNFSQL | |
| | FSQLHLGQIYSLTFNVSTDTAG | HLGQIYSLTFNVSTDTAGMYECV | |
| | MYECVLRNYPHGLIMQRFVILT | LRNYPHGLIMQRFVILTQLETLS | |
| | QLETLSRPDEPCCTPALGRYSL | RPDEPCCTPALGRYSLGDQIWSP | |
| | GDQIWSPTPWRLRNHDCGMYRG | TPWRLRNHDCGMYRGFQRNYFYI | |
| | FQRNYFYIGRADAEDCWKPACP | GRADAEDCWKPACPDEEPDRCWT | |
| | DEEPDRCWTVIQEPELPGDCYR | VIQEPELPGDCYRSQPHPPKFLP | |
| | SQPHPPKFLPVTPAPPADIDTG | VTPAPPADIDTGMSPWATRSGGG | |
| | MSPWATR | GSGGGGSESKYGPPCPPCPAPEF | |
| | | LGGPSVFLFPPKPKDTLMISRTP | |
| | | EVTCVVVDVSQEDPEVQFNWYVD | |
| | | GVEVHNAKTKPREEQFNSTYRVV | |
| | | SVLTVLHQDWLNGKEYKCKVSNK | |
| | | GLPSSIEKTISKAKGQPREPQVY | |
| | | TLPPSQEEMTKNQVSLTCLVKGF | |
| | | YPSDIAVEWESNGQPENNYKTTP | |
| | | PVLDSDGSFFLYSKLTVDKSRWQ | |
| | | EGNVFSCSVMHEALHNHYTQKSL | |
| | | SLSLG | |
| UL141_1 | SFPFATADIAEKMWAENYETTS | SFPFATADIAEKMWAENYETTSP | Fc fusion |
| SEQ ID | PAPVLVAEGEQVTIPCTVMTHS | APVLVAEGEQVTIPCTVMTHSWP | SEQ ID NO: 47 |
| NO: 15 | WPMVSIRARFCRSHDGSDEAIL | MVSIRARFCRSHDGSDEAILNAD | |
| | NADLGHRLMNGLQYRLPYATWN | LGHRLMNGLQYRLPYATWNFSQL | |
| | FSQLHLGQIFSLTFNVSTDTAG | HLGQIFSLTFNVSTDTAGMYECV | |
| | MYECVLRNFPHGLIMQRFVILT | LRNFPHGLIMQRFVILTQLETLS | |
| | QLETLSRPDEPCCTPALGRYSL | RPDEPCCTPALGRYSLGDQIWSP | |
| | GDQIWSPTPWRLRNHDCGMYRG | TPWRLRNHDCGMYRGFQRNYFYI | |
| | FQRNYFYIGRADAEDCWKPACP | GRADAEDCWKPACPDEEPDRCWT | |
| | DEEPDRCWTVIQEYELPGDCYR | VIQEYELPGDCYRSQPHPPKFLP | |
| | SQPHPPKFLPVTPAPPADIDTG | VTPAPPADIDTGMSPWATRSGGG | |
| | MSPWATR | SGGSESKYGPPCPPCPAPEFLGG | |
| | | PSVFLFPPKPKDTLMISRTPEVT | |
| | | CVVVDVSQEDPEVQFNWYVDGVE | |
| | | VHNAKTKPREEQFNSTYRVVSVL | |
| | | TVLHQDWLNGKEYKCKVSNKGLP | |
| | | SSIEKTISKAKGQPREPQVYTLP | |
| | | PSQEEMTKNQVSLTCLVKGFYPS | |
| | | DIAVEWESNGQPENNYKTTPPVL | |
| | | DSDGSFFLYSKLTVDKSRWQEGN | |
| | | VFSCSVMHEALHNHYTQKSLSLS | |
| | | LG | |
| UL141_13 | SFPFATADIAEKMWAENYETTS | SFPFATADIAEKMWAENYETTSP | Fc fusion |
| SEQ ID | PAPVLVAEGEQVTIPCTVMTHS | APVLVAEGEQVTIPCTVMTHSWP | SEQ ID NO: 48 |
| NO: 16 | WPMVSIRARFCRSHDGSDEAIL | MVSIRARFCRSHDGSDEAILNAD | |
| | NADLGHRLMNGLQYRLPYATWN | LGHRLMNGLQYRLPYATWNFSQL | |
| | FSQLHLGQIFSLTFNVSTDTAG | HLGQIFSLTFNVSTDTAGMYECV | |
| | MYECVLRNFPHGLIMQRFVILT | LRNFPHGLIMQRFVILTQLETLS | |
| | QLETLSRPDEPCCTPALGRYSL | RPDEPCCTPALGRYSLGDQIWSP | |
| | GDQIWSPTPWRLRNHDCGMYRG | TPWRLRNHDCGMYRGFQRNYFYI | |
| | FQRNYFYIGRADAEDCWKPACP | GRADAEDCWKPACPDEEPDRCWT | |
| | DEEPDRCWTVIQEPELPGDCYR | VIQEPELPGDCYRSQPHPPKFLP | |
| | SQPHPPKFLPVTPAPPADIDTG | VTPAPPADIDTGMSPWATRSGGG | |
| | MSPWATR | SGGSESKYGPPCPPCPAPEFLGG | |
| | | PSVFLFPPKPKDTLMISRTPEVT | |
| | | CVVVDVSQEDPEVQFNWYVDGVE | |
| | | VHNAKTKPREEQFNSTYRVVSVL | |
| | | TVLHQDWLNGKEYKCKVSNKGLP | |
| | | SSIEKTISKAKGQPREPQVYTLP | |
| | | PSQEEMTKNQVSLTCLVKGFYPS | |
| | | DIAVEWESNGQPENNYKTTPPVL | |
| | | DSDGSFFLYSKLTVDKSRWQEGN | |
| | | VFSCSVMHEALHNHYTQKSLSLS | |
| | | LG | |
| UL141_14 | SFPFATADIAEKMWAENYETTS | SFPFATADIAEKMWAENYETTSP | Fc fusion |
| SEQ ID | PAPVLVAEGEQVTIPCTVMTHS | APVLVAEGEQVTIPCTVMTHSWP | SEQ ID NO: 49 |
| NO: 17 | WPMVSIRARFCRSHDGSDEAIL | MVSIRARFCRSHDGSDEAILNAD | |
| | NADLGHRLMNGLQYRLPYATWN | LGHRLMNGLQYRLPYATWNFSQL | |
| | FSQLHLGQIFSLTFNVSTDTAG | HLGQIFSLTFNVSTDTAGMYECV | |
| | MYECVLHTYPDGLIMQRFVILT | LHTYPDGLIMQRFVILTQLETLS | |
| | QLETLSRPDEPCCTPALGRYSL | RPDEPCCTPALGRYSLGDQIWSP | |
| | GDQIWSPTPWRLRNHDCGMYRG | TPWRLRNHDCGMYRGFQRNYFYI | |
| | FQRNYFYIGRADAEDCWKPACP | GRADAEDCWKPACPDEEPDRCWT | |
| | DEEPDRCWTVIQDYDLPGDCYR | VIQDYDLPGDCYRSQPHPPKFLP | |
| | SQPHPPKFLPVTPAPPADIDTG | VTPAPPADIDTGMSPWATRSGGG | |
| | MSPWATR | SGGSESKYGPPCPPCPAPEFLGG | |
| | | PSVFLFPPKPKDTLMISRTPEVT | |
| | | CVVVDVSQEDPEVQFNWYVDGVE | |
| | | VHNAKTKPREEQFNSTYRVVSVL | |
| | | TVLHQDWLNGKEYKCKVSNKGLP | |
| | | SSIEKTISKAKGQPREPQVYTLP | |
| | | PSQEEMTKNQVSLTCLVKGFYPS | |
| | | DIAVEWESNGQPENNYKTTPPVL | |
| | | DSDGSFFLYSKLTVDKSRWQEGN | |
| | | VFSCSVMHEALHNHYTQKSLSLS | |
| | | LG | |
| UL141_15 | SFPFATADIAEKMWAENYETTS | SFPFATADIAEKMWAENYETTSP | Fc fusion |
| SEQ ID | PAPVLVAEGEQVTIPCTVMTHS | APVLVAEGEQVTIPCTVMTHSWP | SEQ ID NO: 50 |
| NO: 18 | WPMVSIRARFCRSHDGSDEAIL | MVSIRARFCRSHDGSDEAILNAD | |
| | NADLGHRLMNGLQYRLPYATWN | LGHRLMNGLQYRLPYATWNFSQL | |
| | FSQLHLGQIFSLTFNVSTDTAG | HLGQIFSLTFNVSTDTAGMYECV | |
| | MYECVLHTYPDGLIMQRFVILT | LHTYPDGLIMQRFVILTQLETLS | |
| | QLETLSRPDEPCCTPALGRYSL | RPDEPCCTPALGRYSLGDQIWSP | |
| | GDQIWSPTPWRLRNHDCGMYRG | TPWRLRNHDCGMYRGFQRNYFYI | |
| | FQRNYFYIGRADAEDCWKPACP | GRADAEDCWKPACPDEEPDRCWT | |
| | DEEPDRCWTVIQDDYLPGDCYR | VIQDDYLPGDCYRSQPHPPKFLP | |
| | SQPHPPKFLPVTPAPPADIDTG | VTPAPPADIDTGMSPWATRSGGG | |
| | MSPWATR | SGGSESKYGPPCPPCPAPEFLGG | |
| | | PSVFLFPPKPKDTLMISRTPEVT | |
| | | CVVVDVSQEDPEVQFNWYVDGVE | |
| | | VHNAKTKPREEQFNSTYRVVSVL | |
| | | TVLHQDWLNGKEYKCKVSNKGLP | |
| | | SSIEKTISKAKGQPREPQVYTLP | |
| | | PSQEEMTKNQVSLTCLVKGFYPS | |
| | | DIAVEWESNGQPENNYKTTPPVL | |
| | | DSDGSFFLYSKLTVDKSRWQEGN | |
| | | VFSCSVMHEALHNHYTQKSLSLS | |
| | | LG | |
| UL141_16 | SFPFATADIAEKMWAENYETTS | SFPFATADIAEKMWAENYETTSP | Fc fusion |
| SEQ ID | PAPVLVAEGEQVTIPCTVMTHS | APVLVAEGEQVTIPCTVMTHSWP | SEQ ID NO: 51 |
| NO: 19 | WPMVSIRARFCRSHDGSDELIL | MVSIRARFCRSHDGSDELILDAV | |
| | DAVKGHRLMNGLQYRLPYATWN | KGHRLMNGLQYRLPYATWNFSQL | |
| | FSQLHLGQIFSLTFNVSTDTAG | HLGQIFSLTFNVSTDTAGMYECV | |
| | MYECVLRNYSHGLIMQRFVILT | LRNYSHGLIMQRFVILTLSSGGG | |
| | LSSGGGSPACPDEEPDRCWTVI | SPACPDEEPDRCWTVIQEYELPG | |
| | QEYELPGDCYRSQPHPPKFLPV | DCYRSQPHPPKFLPVTPAPPADI | |
| | TPAPPADIDTGMSPWATR | DTGMSPWATRSGGGSGGSESKYG | |
| | | PPCPPCPAPEFLGGPSVFLFPPK | |
| | | PKDTLMISRTPEVTCVVVDVSQE | |
| | | DPEVQFNWYVDGVEVHNAKTKPR | |
| | | EEQFNSTYRVVSVLTVLHQDWLN | |
| | | GKEYKCKVSNKGLPSSIEKTISK | |
| | | AKGQPREPQVYTLPPSQEEMTKN | |
| | | QVSLTCLVKGFYPSDIAVEWESN | |
| | | GQPENNYKTTPPVLDSDGSFFLY | |
| | | SKLTVDKSRWQEGNVFSCSVMHE | |
| | | ALHNHYTQKSLSLSLG | |
| UL141_17 | SFPFATADIAEKMWAENYETTS | SFPFATADIAEKMWAENYETTSP | Fc fusion |
| SEQ ID | PAPVLVAEGEQVTIPCTVMTHS | APVLVAEGEQVTIPCTVMTHSWP | SEQ ID NO: 52 |
| NO: 20 | WPMVSIRARFCRSHDGSDELIL | MVSIRARFCRSHDGSDELILDAV | |
| | DAVKGHRLMNGLQYRLPYATWN | KGHRLMNGLQYRLPYATWNFSQL | |
| | FSQLHLGQIFSLTENVSTDTAG | HLGQIFSLTFNVSTDTAGMYECV | |
| | MYECVLRNYSHGLIMQRFVILT | LRNYSHGLIMQRFVILTLSSGGG | |
| | LSSGGGSPACPDEEPDRCWTVI | SPACPDEEPDRCWTVIQEPELPG | |
| | QEPELPGDCYRSQPHPPKFLPV | DCYRSQPHPPKFLPVTPAPPADI | |
| | TPAPPADIDTGMSPWATR | DTGMSPWATRSGGGSGGSESKYG | |
| | | PPCPPCPAPEFLGGPSVFLFPPK | |
| | | PKDTLMISRTPEVTCVVVDVSQE | |
| | | DPEVQFNWYVDGVEVHNAKTKPR | |
| | | EEQFNSTYRVVSVLTVLHQDWLN | |
| | | GKEYKCKVSNKGLPSSIEKTISK | |
| | | AKGQPREPQVYTLPPSQEEMTKN | |
| | | QVSLTCLVKGFYPSDIAVEWESN | |
| | | GQPENNYKTTPPVLDSDGSFFLY | |
| | | SKLTVDKSRWQEGNVFSCSVMHE | |
| | | ALHNHYTQKSLSLSLG | |
| UL141_18 | SFPFATADIAEKMWAENYETTS | SFPFATADIAEKMWAENYETTSP | Fc fusion |
| SEQ ID | PAPVLVAEGEQVTIPCTVMTHS | APVLVAEGEQVTIPCTVMTHSWP | SEQ ID NO: 53 |
| NO: 21 | WPMVSIRARFCRSHDGSDELIL | MVSIRARFCRSHDGSDELILDAV | |
| | DAVKGHRLMNGLQYRLPYATWN | KGHRLMNGLQYRLPYATWNFSQL | |
| | FSQLHLGQIFSLTFNVSTDTAG | HLGQIFSLTFNVSTDTAGMYECV | |
| | MYECVLRNYSHGLIMQRFVILT | LRNYSHGLIMQRFVILTLSSGGG | |
| | LSSGGGSPACPDEEPDRCWTVI | SPACPDEEPDRCWTVIQDDYDLP | |
| | QDDYDLPGDCYRSQPHPPKFLP | GDCYRSQPHPPKFLPVTPAPPAD | |
| | VTPAPPADIDTGMSPWATR | IDTGMSPWATRSGGGSGGSESKY | |
| | | GPPCPPCPAPEFLGGPSVFLFPP | |
| | | KPKDTLMISRTPEVTCVVVDVSQ | |
| | | EDPEVQFNWYVDGVEVHNAKTKP | |
| | | REEQFNSTYRVVSVLTVLHQDWL | |
| | | NGKEYKCKVSNKGLPSSIEKTIS | |
| | | KAKGQPREPQVYTLPPSQEEMTK | |
| | | NQVSLTCLVKGFYPSDIAVEWES | |
| | | NGQPENNYKTTPPVLDSDGSFFL | |
| | | YSKLTVDKSRWQEGNVFSCSVMH | |
| | | EALHNHYTQKSLSLSLG | |
| UL141_19 | SFPFATADIAEKMWAENYETTS | SFPFATADIAEKMWAENYETTSP | Fc fusion |
| SEQ ID | PAPVLVAEGEQVTIPCTVMTHS | APVLVAEGEQVTIPCTVMTHSWP | SEQ ID NO: 54 |
| NO: 22 | WPMVSIRARFCRSHDGSDELIL | MVSIRARFCRSHDGSDELILDAV | |
| | DAVKGHRLMNGLQYRLPYATWN | KGHRLMNGLQYRLPYATWNFSQL | |
| | FSQLHLGQIFSLTFNVSTDTAG | HLGQIFSLTFNVSTDTAGMYECV | |
| | MYECVLRNYSHGLIMQRFVILT | LRNYSHGLIMQRFVILTLSSGGG | |
| | LSSGGGSPACPDEEPDRCWTVI | SPACPDEEPDRCWTVIQDDPDLP | |
| | QDDPDLPGDCYRSQPHPPKFLP | GDCYRSQPHPPKFLPVTPAPPAD | |
| | VTPAPPADIDTGMSPWATR | IDTGMSPWATRSGGGSGGSESKY | |
| | | GPPCPPCPAPEFLGGPSVFLFPP | |
| | | KPKDTLMISRTPEVTCVVVDVSQ | |
| | | EDPEVQFNWYVDGVEVHNAKTKP | |
| | | REEQFNSTYRVVSVLTVLHQDWL | |
| | | NGKEYKCKVSNKGLPSSIEKTIS | |
| | | KAKGQPREPQVYTLPPSQEEMTK | |
| | | NQVSLTCLVKGFYPSDIAVEWES | |
| | | NGQPENNYKTTPPVLDSDGSFFL | |
| | | YSKLTVDKSRWQEGNVFSCSVMH | |
| | | EALHNHYTQKSLSLSLG | |
| UL141_20 | SFPFATADIAEKMWAENYETTS | SFPFATADIAEKMWAENYETTSP | Fc fusion |
| SEQ ID | PAPVLVAEGEQVTIPCTVMTHS | APVLVAEGEQVTIPCTVMTHSWP | SEQ ID NO: 55 |
| NO: 23 | WPMVSIRARFCRSHDGSDELIL | MVSIRARFCRSHDGSDELILDAV | |
| | DAVKGHRLMNGLQYRLPYATWN | KGHRLMNGLQYRLPYATWNFSQL | |
| | FSQLHLGQIFSLTFNVSTDTAG | HLGQIFSLTFNVSTDTAGMYECV | |
| | MYECVLRNYSHGLIMQRFVILT | LRNYSHGLIMQRFVILTQLETLS | |
| | QLETLSRPDEPCCTPALGRYSL | RPDEPCCTPALGRYSLGDQIWSP | |
| | GDQIWSPTPWRLRNHDCGMYRG | TPWRLRNHDCGMYRGFQRNYFYI | |
| | FQRNYFYIGRADAEDCWKPACP | GRADAEDCWKPACPDEEPDRCWT | |
| | DEEPDRCWTVIQDYDLPGDCYR | VIQDYDLPGDCYRSQPHPPKFLP | |
| | SQPHPPKFLPVTPAPPADIDTG | VTPAPPADIDTGMSPWATRSGGG | |
| | MSPWATR | SGGSESKYGPPCPPCPAPEFLGG | |
| | | PSVFLFPPKPKDTLMISRTPEVT | |
| | | CVVVDVSQEDPEVQFNWYVDGVE | |
| | | VHNAKTKPREEQFNSTYRVVSVL | |
| | | TVLHQDWLNGKEYKCKVSNKGLP | |
| | | SSIEKTISKAKGQPREPQVYTLP | |
| | | PSQEEMTKNQVSLTCLVKGFYPS | |
| | | DIAVEWESNGQPENNYKTTPPVL | |
| | | DSDGSFFLYSKLTVDKSRWQEGN | |
| | | VFSCSVMHEALHNHYTQKSLSLS | |
| | | LG | |
| UL141_21 | SFPFATADIAEKMWAENYETTS | SFPFATADIAEKMWAENYETTSP | Fc fusion |
| SEQ ID | PAPVLVAEGEQVTIPCTVMTHS | APVLVAEGEQVTIPCTVMTHSWP | SEQ ID NO: 56 |
| NO: 24 | WPMVSIRARFCRSHDGSDELIL | MVSIRARFCRSHDGSDELILDAV | |
| | DAVKGHRLMNGLQYRLPYATWN | KGHRLMNGLQYRLPYATWNFSQL | |
| | FSQLHLGQIFSLTFNVSTDTAG | HLGQIFSLTFNVSTDTAGMYECV | |
| | MYECVLRNYSHGLIMQRFVILT | LRNYSHGLIMQRFVILTQLETLS | |
| | QLETLSRPDEPCCTPALGRYSL | RPDEPCCTPALGRYSLGDQIWSP | |
| | GDQIWSPTPWRLRNHDCGMYRG | TPWRLRNHDCGMYRGFQRNYFYI | |
| | FQRNYFYIGRADAEDCWKPACP | GRADAEDCWKPACPDEEPDRCWT | |
| | DEEPDRCWTVDEGSDELPGDCY | VDEGSDELPGDCYESQPHPPKFL | |
| | ESQPHPPKFLPVTPAPPADIDT | PVTPAPPADIDTGMSPWATRSGG | |
| | GMSPWATR | GSGGSESKYGPPCPPCPAPEFLG | |
| | | GPSVFLFPPKPKDTLMISRTPEV | |
| | | TCVVVDVSQEDPEVQFNWYVDGV | |
| | | EVHNAKTKPREEQFNSTYRVVSV | |
| | | LTVLHQDWLNGKEYKCKVSNKGL | |
| | | PSSIEKTISKAKGQPREPQVYTL | |
| | | PPSQEEMTKNQVSLTCLVKGFYP | |
| | | SDIAVEWESNGQPENNYKTTPPV | |
| | | LDSDGSFFLYSKLTVDKSRWQEG | |
| | | NVFSCSVMHEALHNHYTQKSLSL | |
| | | SLG | |
| UL141_22 | SFPFATADIAEKMWAENYETTS | SFPFATADIAEKMWAENYETTSP | Fc fusion |
| SEQ ID | PAPVLVAEGEQVTIPCTVMTHS | APVLVAEGEQVTIPCTVMTHSWP | SEQ ID NO: 57 |
| NO: 25 | WPMVSIRARFCRSHDGSDELIL | MVSIRARFCRSHDGSDELILDAV | |
| | DAVKGHRLMNGLQYRLPYATWN | KGHRLMNGLQYRLPYATWNFSQL | |
| | FSQLHLGQIFSLTFNVSTDTAG | HLGQIFSLTFNVSTDTAGMYECV | |
| | MYECVLRNYSHGLIMQRFVILT | LRNYSHGLIMQRFVILTQLETLS | |
| | QLETLSRPDEPCCTPALGRYSL | RPDEPCCTPALGRYSLGDQIWSP | |
| | GDQIWSPTPWRLRNHDCGMYRG | TPWRLRNHDCGMYRGFQRNYFYI | |
| | FQRNYFYIGRADAEDCWKPACP | GRADAEDCWKPACPDEEPDRCWT | |
| | DEEPDRCWTVIESDELPGDCYE | VIESDELPGDCYESQPHPPKFLP | |
| | SQPHPPKFLPVTPAPPADIDTG | VTPAPPADIDTGMSPWATRSGGG | |
| | MSPWATR | SGGSESKYGPPCPPCPAPEFLGG | |
| | | PSVFLFPPKPKDTLMISRTPEVT | |
| | | CVVVDVSQEDPEVQFNWYVDGVE | |
| | | VHNAKTKPREEQFNSTYRVVSVL | |
| | | TVLHQDWLNGKEYKCKVSNKGLP | |
| | | SSIEKTISKAKGQPREPQVYTLP | |
| | | PSQEEMTKNQVSLTCLVKGFYPS | |
| | | DIAVEWESNGQPENNYKTTPPVL | |
| | | DSDGSFFLYSKLTVDKSRWQEGN | |
| | | VFSCSVMHEALHNHYTQKSLSLS | |
| | | LG | |
| UL141_23 | SFPFATADIAEKMWAENYETTS | EVQLVESGGGVVQPGRSLRLSCS | Farletuzumab |
| SEQ ID | PAPVLVAEGEQVTIPCTVMTHS | ASGFTFSGYGLSWVRQAPGKGLE | fusion |
| NO: 26 | WPMVSIRARFCRSHDGSDEAIL | WVAMISSGGSYTYYADSVKGRFA | SEQ ID NO: 58 |
| | NADLGHRLMNGLQYRLPYATWN | ISRDNAKNTLFLQMDSLRPEDTG | |
| | FSQLHLGQIFSLTFNVSTDTAG | VYFCARHGDDPAWFAYWGQGTPV | |
| | MYECVLRNFPHGLIMQRFVILT | TVSSASTKGPSVFPLAPSSKSTS | |
| | QLESRPDEPCCTPALGRYSLGD | GGTAALGCLVKDYFPEPVTVSWN | |
| | QIWSPTPWRLRNHDCGMYRGES | SGALTSGVHTFPAVLQSSGLYSL | |
| | | SSVVTVPSSSLGTQTYICNVNHK | |
| | | PSNTKVDKKVEPKSCDKTHTCPP | |
| | | CPAPELLGGPSVFLFPPKPKDTL | |
| | | MISRTPEVTCVVVDVEHEDPEVK | |
| | | FNWYVDGVEVHNAKTKPREEQYN | |
| | | STYRVVSVLTVLHQDWLNGKEYK | |
| | | CKVSNKALPAPIEKTISKAKGQP | |
| | | REPQVYTLPPSREEMTKNQVSLT | |
| | | CLVKGFYPSDIAVEWESNGQPEN | |
| | | NYKTTPPVLDSDGSFFLYSKLTV | |
| | | DKSRWQQGNVFSCSVMHEALHNH | |
| | | YTQKSLSLSLGKGGGSGGGSSFP | |
| | | FATADIAEKMWAENYETTSPAPV | |
| | | LVAEGEQVTIPCTVMTHSWPMVS | |
| | | IRARFCRSHDGSDEAILNADLGH | |
| | | RLMNGLQYRLPYATWNFSQLHLG | |
| | | QIFSLTFNVSTDTAGMYECVLRN | |
| | | FPHGLIMQRFVILTQLESRPDEP | |
| | | CCTPALGRYSLGDQIWSPTPWRL | |
| | | RNHDCGMYRGFS | |
| UL141_24 | SFPFATADIAEKMWAENYETTS | EVQLVESGGGVVQPGRSLRLSCS | Farletuzumab |
| SEQ ID | PAPVLVAEGEQVTIPCTVMTHS | ASGFTFSGYGLSWVRQAPGKGLE | fusion |
| NO: 27 | WPMVSIRARFCRSHDGSDEAIL | WVAMISSGGSYTYYADSVKGRFA | SEQ ID NO: 59 |
| | NADLGHRLMNGLQYRLPYATWN | ISRDNAKNTLFLQMDSLRPEDTG | |
| | FSQLHLGQIFSLTFNVSTDTAG | VYFCARHGDDPAWFAYWGQGTPV | |
| | MYECVLRNFPHGLIMQRFVILT | TVSSASTKGPSVFPLAPSSKSTS | |
| | QLESRPDEPCCTPALGRYSLGD | GGTAALGCLVKDYFPEPVTVSWN | |
| | QIWSPTPWRLRNHDCGMYRGFQ | SGALTSGVHTFPAVLQSSGLYSL | |
| | RNYFYIGRA | SSVVTVPSSSLGTQTYICNVNHK | |
| | | PSNTKVDKKVEPKSCDKTHTCPP | |
| | | CPAPELLGGPSVFLFPPKPKDTL | |
| | | MISRTPEVTCVVVDVEHEDPEVK | |
| | | FNWYVDGVEVHNAKTKPREEQYN | |
| | | STYRVVSVLTVLHQDWLNGKEYK | |
| | | CKVSNKALPAPIEKTISKAKGQP | |
| | | REPQVYTLPPSREEMTKNQVSLT | |
| | | CLVKGFYPSDIAVEWESNGQPEN | |
| | | NYKTTPPVLDSDGSFFLYSKLTV | |
| | | DKSRWQQGNVFSCSVMHEALHNH | |
| | | YTQKSLSLSLGKGGGSGGGSSFP | |
| | | FATADIAEKMWAENYETTSPAPV | |
| | | LVAEGEQVTIPCTVMTHSWPMVS | |
| | | IRARFCRSHDGSDEAILNADLGH | |
| | | RLMNGLQYRLPYATWNFSQLHLG | |
| | | QIFSLTFNVSTDTAGMYECVLRN | |
| | | FPHGLIMQRFVILTQLESRPDEP | |
| | | CCTPALGRYSLGDQIWSPTPWRL | |
| | | RNHDCGMYRGFQRNYFYIGRA | |
| UL141_25 | SFPFATADIAEKMWAENYETTS | EVQLVESGGGVVQPGRSLRLSCS | Farletuzumab |
| SEQ ID | PAPVLVAEGEQVTIPCTVMTHS | ASGFTFSGYGLSWVRQAPGKGLE | fusion |
| NO: 28 | WPMVSIRARFCRSHDGSDEAIL | WVAMISSGGSYTYYADSVKGRFA | SEQ ID NO: 60 |
| | NADLGHRLMNGLQYRLPYATWN | ISRDNAKNTLFLQMDSLRPEDTG | |
| | FSQLHLGQIFSLTFNVSTDTAG | VYFCARHGDDPAWFAYWGQGTPV | |
| | MYECVLRNFPHGLIMQRFVILT | TVSSASTKGPSVFPLAPSSKSTS | |
| | QLETLSRPDEPCCTPALGRYSL | GGTAALGCLVKDYFPEPVTVSWN | |
| | GDQIWSPTPWRLRNHDCGMYRG | SGALTSGVHTFPAVLQSSGLYSL | |
| | FQRNYFYIGRADAEDCWKPACP | SSVVTVPSSSLGTQTYICNVNHK | |
| | S | PSNTKVDKKVEPKSCDKTHTCPP | |
| | | CPAPELLGGPSVFLFPPKPKDTL | |
| | | MISRTPEVTCVVVDVEHEDPEVK | |
| | | FNWYVDGVEVHNAKTKPREEQYN | |
| | | STYRVVSVLTVLHQDWLNGKEYK | |
| | | CKVSNKALPAPIEKTISKAKGQP | |
| | | REPQVYTLPPSREEMTKNQVSLT | |
| | | CLVKGFYPSDIAVEWESNGQPEN | |
| | | NYKTTPPVLDSDGSFFLYSKLTV | |
| | | DKSRWQQGNVFSCSVMHEALHNH | |
| | | YTQKSLSLSLGKGGGSGGGSSFP | |
| | | FATADIAEKMWAENYETTSPAPV | |
| | | LVAEGEQVTIPCTVMTHSWPMVS | |
| | | IRARFCRSHDGSDEAILNADLGH | |
| | | RLMNGLQYRLPYATWNFSQLHLG | |
| | | QIFSLTFNVSTDTAGMYECVLRN | |
| | | FPHGLIMQRFVILTQLETLSRPD | |
| | | EPCCTPALGRYSLGDQIWSPTPW | |
| | | RLRNHDCGMYRGFQRNYFYIGRA | |
| | | DAEDCWKPACPS | |
| UL141_26: | SFPFATADIAEKMWAENYETTS | EVQLVESGGGVVQPGRSLRLSCS | Farletuzumab |
| SEQ ID | PAPVLVAEGEQVTIPCTVMTHS | ASGFTFSGYGLSWVRQAPGKGLE | fusion |
| NO: 29 | WPMVSIRARFCRSHDGSDEAIL | WVAMISSGGSYTYYADSVKGRFA | SEQ ID NO: 61 |
| | NADLGHRLMNGLQYRLPYATWN | ISRDNAKNTLFLQMDSLRPEDTG | |
| | FSQLHLGQIFSLTFNVSTDTAG | VYFCARHGDDPAWFAYWGQGTPV | |
| | MYECVLRNFPHGLIMQRFVILT | TVSSASTKGPSVFPLAPSSKSTS | |
| | QLESRPDEPCCTPALGRYSLGD | GGTAALGCLVKDYFPEPVTVSWN | |
| | QIWSPTPWRLRNHDCGMYRGES | SGALTSGVHTFPAVLQSSGLYSL | |
| | | SSVVTVPSSSLGTQTYICNVNHK | |
| | | PSNTKVDKKVEPKSCDKTHTCPP | |
| | | CPAPELLGGPSVFLFPPKPKDTL | |
| | | MISRTPEVTCVVVDVEHEDPEVK | |
| | | FNWYVDGVEVHNAKTKPREEQYN | |
| | | STYRVVSVLTVLHQDWLNGKEYK | |
| | | CKVSNKALPAPIEKTISKAKGQP | |
| | | REPQVYTLPPSREEMTKNQVSLT | |
| | | CLVKGFYPSDIAVEWESNGQPEN | |
| | | NYKTTPPVLDSDGSFFLYSKLTV | |
| | | DKSRWQQGNVFSCSVMHEALHNH | |
| | | YTQKSLSLSLGKGAAAGAAASFP | |
| | | FATADIAEKMWAENYETTSPAPV | |
| | | LVAEGEQVTIPCTVMTHSWPMVS | |
| | | IRARFCRSHDGSDEAILNADLGH | |
| | | RLMNGLQYRLPYATWNFSQLHLG | |
| | | QIFSLTFNVSTDTAGMYECVLRN | |
| | | FPHGLIMQRFVILTQLESRPDEP | |
| | | CCTPALGRYSLGDQIWSPTPWRL | |
| | | RNHDCGMYRGFS | |
| UL141_27 | SFPFATADIAEKMWAENYETTS | EVQLVESGGGVVQPGRSLRLSCS | Farletuzumab |
| SEQ ID | PAPVLVAEGEQVTIPCTVMTHS | ASGFTFSGYGLSWVRQAPGKGLE | fusion |
| NO: 30 | WPMVSIRARFCRSHDGSDEAIL | WVAMISSGGSYTYYADSVKGRFA | SEQ ID NO: 62 |
| | NADLGHRLMNGLQYRLPYATWN | ISRDNAKNTLFLQMDSLRPEDTG | |
| | FSQLHLGQIFSLTFNVSTDTAG | VYFCARHGDDPAWFAYWGQGTPV | |
| | MYECVLRNFPHGLIMQRFVILT | TVSSASTKGPSVFPLAPSSKSTS | |
| | QLESRPDEPCCTPALGRYSLGD | GGTAALGCLVKDYFPEPVTVSWN | |
| | QIWSPTPWRLRNHDCGMYRGFQ | SGALTSGVHTFPAVLQSSGLYSL | |
| | RNYFYIGRA | SSVVTVPSSSLGTQTYICNVNHK | |
| | | PSNTKVDKKVEPKSCDKTHTCPP | |
| | | CPAPELLGGPSVFLFPPKPKDTL | |
| | | MISRTPEVTCVVVDVEHEDPEVK | |
| | | FNWYVDGVEVHNAKTKPREEQYN | |
| | | STYRVVSVLTVLHQDWLNGKEYK | |
| | | CKVSNKALPAPIEKTISKAKGQP | |
| | | REPQVYTLPPSREEMTKNQVSLT | |
| | | CLVKGFYPSDIAVEWESNGQPEN | |
| | | NYKTTPPVLDSDGSFFLYSKLTV | |
| | | DKSRWQQGNVFSCSVMHEALHNH | |
| | | YTQKSLSLSLGKGAAAGAAASFP | |
| | | FATADIAEKMWAENYETTSPAPV | |
| | | LVAEGEQVTIPCTVMTHSWPMVS | |
| | | IRARFCRSHDGSDEAILNADLGH | |
| | | RLMNGLQYRLPYATWNFSQLHLG | |
| | | QIFSLTFNVSTDTAGMYECVLRN | |
| | | FPHGLIMQRFVILTQLESRPDEP | |
| | | CCTPALGRYSLGDQIWSPTPWRL | |
| | | RNHDCGMYRGFQRNYFYIGRA | |
| UL141_28 | SFPFATADIAEKMWAENYETTS | EVQLVESGGGVVQPGRSLRLSCS | Farletuzumab |
| SEQ ID | PAPVLVAEGEQVTIPCTVMTHS | ASGFTFSGYGLSWVRQAPGKGLE | fusion |
| NO: 31 | WPMVSIRARFCRSHDGSDEAIL | WVAMISSGGSYTYYADSVKGRFA | SEQ ID NO: 63 |
| | NADLGHRLMNGLQYRLPYATWN | ISRDNAKNTLFLQMDSLRPEDTG | |
| | FSQLHLGQIFSLTFNVSTDTAG | VYFCARHGDDPAWFAYWGQGTPV | |
| | MYECVLRNFPHGLIMQRFVILT | TVSSASTKGPSVFPLAPSSKSTS | |
| | QLETLSRPDEPCCTPALGRYSL | GGTAALGCLVKDYFPEPVTVSWN | |
| | GDQIWSPTPWRLRNHDCGMYRG | SGALTSGVHTFPAVLQSSGLYSL | |
| | FQRNYFYIGRADAEDCWKPACP | SSVVTVPSSSLGTQTYICNVNHK | |
| | S | PSNTKVDKKVEPKSCDKTHTCPP | |
| | | CPAPELLGGPSVFLFPPKPKDTL | |
| | | MISRTPEVTCVVVDVEHEDPEVK | |
| | | FNWYVDGVEVHNAKTKPREEQYN | |
| | | STYRVVSVLTVLHQDWLNGKEYK | |
| | | CKVSNKALPAPIEKTISKAKGQP | |
| | | REPQVYTLPPSREEMTKNQVSLT | |
| | | CLVKGFYPSDIAVEWESNGQPEN | |
| | | NYKTTPPVLDSDGSFFLYSKLTV | |
| | | DKSRWQQGNVFSCSVMHEALHNH | |
| | | YTQKSLSLSLGKGAAAGAAASFP | |
| | | FATADIAEKMWAENYETTSPAPV | |
| | | LVAEGEQVTIPCTVMTHSWPMVS | |
| | | IRARFCRSHDGSDEAILNADLGH | |
| | | RLMNGLQYRLPYATWNFSQLHLG | |
| | | QIFSLTFNVSTDTAGMYECVLRN | |
| | | FPHGLIMQRFVILTQLETLSRPD | |
| | | EPCCTPALGRYSLGDQIWSPTPW | |
| | | RLRNHDCGMYRGFQRNYFYIGRA | |
| | | DAEDCWKPACPS | |
| UL141_31 | SFPFATADIAEKMWAENYETTS | SFPFATADIAEKMWAENYETTSP | Lexatumumab |
| UL141_D11 | PAPVLVAEGEQVTIPCTVMTHS | APVLVAEGEQVTIPCTVMTHSWP | fusion |
| (2) | WPMVSIRARFCRSHDGSDELIL | MVSIRARFCRSHDGSDELILNAD | SEQ ID NO: 64 |
| Lexa | NADLGHRLMNGLQYRLPFATWN | LGHRLMNGLQYRLPFATWNFSQL | |
| SEQ ID | FSQLHLGQIYSLTFNVSTDTAG | HLGQIYSLTFNVSTDTAGMYECV | |
| NO: 32 | MYECVLRNYPHGLIMQRFVILT | LRNYPHGLIMQRFVILTQLETLS | |
| | QLETLSRPDEPCCTPALGRYSL | RPDEPCCTPALGRYSLGDQIWSP | |
| | GDQIWSPTPWRLRNHDCGMYRG | TPWRLRNHDCGMYRGFQRNYFYI | |
| | FQRNYFYIGRADAEDCWKPACP | GRADAEDCWKPACPDEEPDRCWT | |
| | DEEPDRCWTVIQEPELPGDCYR | VIQEPELPGDCYRSQPHPPKFLP | |
| | SQPHPPKFLPVTPAPPADIDTG | VTPAPPADIDTGMSPWATRSGAA | |
| | MSPWATR | AGAAAESKYGPPCPPCPAPEFLG | |
| | | GPSVFLFPPKPKDTLMISRTPEV | |
| | | TCVVVDVSQEDPEVQFNWYVDGV | |
| | | EVHNAKTKPREEQFNSTYRVVSV | |
| | | LTVLHQDWLNGKEYKCKVSNKGL | |
| | | PSSIEKTISKAKGQPREPQVYTL | |
| | | PPSQEEMTKNQVSLTCLVKGFYP | |
| | | SDIAVEWESNGQPENNYKTTPPV | |
| | | LDSDGSFFLYSKLTVDKSRWQEG | |
| | | NVFSCSVMHEALHNHYTQKSLSL | |
| | | SLGGGSGGSSSELTQDPAVSVAL | |
| | | GQTVRITCQGDSLRSYYASWYQQ | |
| | | KPGQAPVLVIYGKNNRPSGIPDR | |
| | | FSGSSSGNTASLTITGAQAEDEA | |
| | | DYYCNSRDSSGNHVVFGGGTKLT | |
| | | VLGGGGSGGGDSGGGGSGGGGSE | |
| | | VQLVQSGGGVERPGGSLRLSCAA | |
| | | SGFTFDDYGMSWVRQAPGKGLEW | |
| | | VSGINWNGGSTGYADSVKGRVTI | |
| | | SRDNAKNSLYLQMNSLRAEDTAV | |
| | | YYCAKILGAGRGWYFDLWGKGTT | |
| | | VTVSS | |
| UL141_32 | SFPFATADIAEKMWAENYETTS | SFPFATADIAEKMWAENYETTSP | Lexatumumab |
| UL141_D11 | PAPVLVAEGEQVTIPCTVMTHS | APVLVAEGEQVTIPCTVMTHSWP | fusion |
| (2A) Lexa | WPMVSIRARFCRSHDGSDELIL | MVSIRARFCRSHDGSDELILNAD | SEQ ID NO: 65 |
| SEQ ID | NADLGHRLMNGLQYRLPFATWN | LGHRLMNGLQYRLPFATWNFSQL | |
| NO: 33 | FSQLHLGQIYSLTFNVSTDTAG | HLGQIYSLTFNVSTDTAGMYECV | |
| | MYECVLRNYPHGLIMQRFVILT | LRNYPHGLIMQRFVILTQLETLS | |
| | QLETLSRPDEPCCTPALGRYSL | RPDEPCCTPALGRYSLGDQIWSP | |
| | GDQIWSPTPWRLRNHDCGMYRG | TPWRLRNHDCGMYRGFQRNYFYI | |
| | FQRNYFYIGRADAEDCWKPACP | GRADAEDCWKPACPDEEPDRCWT | |
| | DEEPDRCWTVIQEPELPGDCYR | VIQEPELPGDCYRSQPHPPKFLP | |
| | SQPHPPKFLPVTPAPPADIDTG | VTPAPPADIDTGMSPWATRSGGG | |
| | MSPWATR | GSGGGGSESKYGPPCPPCPAPEF | |
| | | LGGPSVFLFPPKPKDTLMISRTP | |
| | | EVTCVVVDVSQEDPEVQFNWYVD | |
| | | GVEVHNAKTKPREEQFNSTYRVV | |
| | | SVLTVLHQDWLNGKEYKCKVSNK | |
| | | GLPSSIEKTISKAKGQPREPQVY | |
| | | TLPPSQEEMTKNQVSLTCLVKGF | |
| | | YPSDIAVEWESNGQPENNYKTTP | |
| | | PVLDSDGSFFLYSKLTVDKSRWQ | |
| | | EGNVFSCSVMHEALHNHYTQKSL | |
| | | SLSLGGSGGSSSELTQDPAVSVA | |
| | | LGQTVRITCQGDSLRSYYASWYQ | |
| | | QKPGQAPVLVIYGKNNRPSGIPD | |
| | | RFSGSSSGNTASLTITGAQAEDE | |
| | | ADYYCNSRDSSGNHVVFGGGTKL | |
| | | TVLGGGGSGGGDSGGGGSGGGGS | |
| | | EVQLVQSGGGVERPGGSLRLSCA | |
| | | ASGFTFDDYGMSWVRQAPGKGLE | |
| | | WVSGINWNGGSTGYADSVKGRVT | |
| | | ISRDNAKNSLYLQMNSLRAEDTA | |
| | | VYYCAKILGAGRGWYFDLWGKGT | |
| | | TVTVSS | |

All publications, patents and patent applications cited in this specification are herein incorporated by reference as if each individual publication or patent application were specifically and individually indicated to be incorporated by reference.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of embodiments disclosed herein includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the present disclosure.

What is claimed:

1. A UL141 variant, wherein the UL141 variant comprises at least one mutation relative to a control UL141 polypeptide having an amino acid sequence according to SEQ ID NO:1, wherein the at least one mutation is selected from the group consisting of: L93A, D96N, V98D, K99L, Y113F, F127Y, R146H, N147T, S149P, H150D, R240E, Y241P, R242E, and combinations thereof.

2. The UL141 variant of claim 1, wherein the UL141 variant comprises the mutations R240E and R242E.

3. The UL141 variant of claim 1, wherein the UL141 variant comprises the mutations R240E, Y241P, and R242E.

4. The UL141 variant of claim 1, wherein the UL141 variant comprises at least one mutation selected from the group consisting of: L93A, D96N, V98D, K99L, Y113F, R146H, N147T, S149P, H150D, R240E, R242E, and combinations thereof.

5. The UL141 variant of claim 2, wherein the UL141 variant comprises each of the following mutations: L93A, D96N, V98D, K99L, Y113F, R146H, N147T, S149P, H150D, R240E, and R242E.

6. The UL141 variant of claim 1, wherein the UL141 variant comprises at least one mutation selected from the group consisting of: D96N, V98D, K99L, F127Y, S149P, R240E, Y241P, R242E, and combinations thereof.

7. The UL141 variant of claim 4, wherein the UL141 variant comprises each of the following mutations: D96N, V98D, K99L, F127Y, S149P, R240E, Y241P, and R242E.

8. The UL141 variant of claim 1, wherein the UL141 variant further comprises a mutation selected from the group consisting of Q162L, T165del, L166del, Q207S, I238D, Q239E, R240D, R240G, R240S, Y241D, Y241S, R242D, R242Y, R242P, 242-insD-243, 242-insE-243, R251E, and combinations thereof.

9. The UL141 variant of claim 1, wherein the UL141 variant further comprises a mutation selected from the group consisting of 30-161del, 173-279del, 183-279del, 193-279del, 203-279del, 213-279del, 223-279del, 233-279del, 218-223del, 271-279del, 163-279del, 208-279del, 217-279del, and 228-279del.

10. The UL141 variant of any one of claims 1-9, wherein the UL141 variant has at least 90% sequence identity to SEQ ID NO:1.

11. The UL141 variant of any one of claims 1-10, wherein the UL141 variant has increased activation of DR5.

12. The UL141 variant of the any one of claims 1-11, wherein the at least one mutation is on DR5 contacting surface of the UL141 variant.

13. The UL141 variant of any one of claims 1-12, wherein the UL141 variant has increased inhibition of CD155 activity or CD155-TIGIT activity.

14. The UL141 variant of the any one of claims 1-13 wherein the at least one mutation is on CD155 contacting surface of the UL141 variant.

15. A UL141 variant according to any one of SEQ ID NOS:2-33.

16. A fusion protein comprising the UL141 variant of any one of claims 1-15.

17. The fusion protein of claim 16 that comprises an antibody sequence or an antibody fragment sequence.

18. The fusion protein of claim 17, wherein the antibody fragment is an Fc fragment.

19. The fusion protein of claim 18, wherein the antibody is farletuzumab or lexatumumab.

20. The fusion protein of claim 16 comprising CD28, IX40/4-1BB, and/or CD3-zeta.

21. A fusion protein according to any one of SEQ ID NOS:34-65.

22. A bispecific macromolecule comprising the UL141 variant of any of the preceding claims.

23. The bispecific macromolecule of claim 22 that comprises a second binding moiety with specificity for a cancer antigen, for instance FOLR1.

24. A nucleic acid encoding the UL141 variant or fusion or bispecific macromolecule of any of claims 1-23.

25. A composition comprising the UL141 variant or fusion or bispecific macromolecule of any of claims 1-23 and a pharmaceutically acceptable carrier or excipient.

26. The composition of claim 25, wherein the composition is used for treating cancer.

27. A method for treating a disease, the method comprising administering a therapeutically effective amount of a UL141 variant or fusion or bispecific macromolecule of any of claims 1-23, or the composition of claim 25, to a subject in need thereof.

28. The method of claim 27, wherein the disease is cancer.

29. The method of claim 28, wherein the cancer is a solid tumor.

30. The method of claim 28, wherein the cancer is ovarian cancer.

31. The method of claim 27, further comprising administering one or more additional agents effective to treat the disease.


SEQUENCE LISTING (66 sequences)

SEQ ID NO: 1 - protein, 249 aa, Homo sapiens
  REGION 1..249: note = misc_feature - mature form UL141
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLETLSRPDEPCCTPALGRYSLGDQIWSPTPWRLRNHDCGMYRGFQRNYFYIGRADAEDCWKPACPDEEPDRCWTVIQRYRLPGDCYRSQPHPPKFLPVTPAPPADIDTGMSPWATR

SEQ ID NO: 2 - protein, 135 aa, synthetic construct
  REGION 1..135: note = Synthetic: UL141_1 30-162
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLE

SEQ ID NO: 3 - protein, 117 aa, synthetic construct
  REGION 1..117: note = Synthetic: UL141_2 162-279
QLETLSRPDEPCCTPALGRYSLGDQIWSPTPWRLRNHDCGMYRGFQRNYFYIGRADAEDCWKPACPDEEPDRCWTVIQRYRLPGDCYRSQPHPPKFLPVTPAPPADIDTGMSPWATR

SEQ ID NO: 4 - protein, 145 aa, synthetic construct
  REGION 1..145: note = Synthetic: UL141_3 30-172
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLESRPDEPCCTP

SEQ ID NO: 5 - protein, 155 aa, synthetic construct
  REGION 1..155: note = Synthetic: UL141_4 30-182
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLESRPDEPCCTPALGRYSLGDQ

SEQ ID NO: 6 - protein, 155 aa, synthetic construct; identical to SEQ ID NO: 5
  REGION 1..155: note = Synthetic: UL141_5 30-192

SEQ ID NO: 7 - protein, 165 aa, synthetic construct
  REGION 1..165: note = Synthetic: UL141_6 30-202
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLESRPDEPCCTPALGRYSLGDQIWSPTPWRLR

SEQ ID NO: 8 - protein, 175 aa, synthetic construct
  REGION 1..175: note = Synthetic: UL141_7 30-212
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLESRPDEPCCTPALGRYSLGDQIWSPTPWRLRNHDCGMYRGF

SEQ ID NO: 9 - protein, 185 aa, synthetic construct
  REGION 1..185: note = Synthetic: UL141_8 30-222
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLESRPDEPCCTPALGRYSLGDQIWSPTPWRLRNHDCGMYRGFQRNYFYIGRA

SEQ ID NO: 10 - protein, 198 aa, synthetic construct
  REGION 1..198: note = Synthetic: UL141_9 30-232
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLETLSRPDEPCCTPALGRYSLGDQIWSPTPWRLRNHDCGMYRGFQRNYFYIGRADAEDCWKPACP

SEQ ID NO: 11 - protein, 194 aa, synthetic construct
  REGION 1..194: note = Synthetic: UL141_10 30-217-224-270
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTLSSGGGSPACPDEEPDRCWTVIQEYELPGDCYRSQPHPPKFLPVTPAPPADIDTGMSPWATR

SEQ ID NO: 12 - protein, 249 aa, synthetic construct
  REGION 1..249: note = Synthetic: UL141_11
SFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLHTYPDGLIMQRFVILTQLETLSRPDEPCCTPALGRYSLGDQIWSPTPWRLRNHDCGMYRGFQRNYFYIGRADAEDCWKPACPDEEPDRCWTVIQEYELPGDCYRSQPHPPKFLPVTPAPPADIDTGMSPWATR

SEQ ID NO: 13 - protein, 249 aa, synthetic construct
  REGION 1..249: note = Synthetic: UL141_11(2)
SFPFATADIAEKMWAENYETTSPAPVLVAEGEQVTIPCTVMTHSWPMVSIRARFCRSHDGSDELILNADLGHRLMNGLQYRLPFATWNFSQLHLGQIYSLTFNVSTDTAGMYECVLRNYPHGLIMQRFVILTQLETLSRPDEPCCTPALGRYSLGDQIWSPTPWRLRNHDCGMYRGFQRNYFYIGRADAEDCWKPACPDEEPDRCWTVIQEPELPGDCYRSQPHPPKFLPVTPAPPADIDTGMSPWATR

SEQ ID NO: 14 - protein, 249 aa, synthetic construct; identical to SEQ ID NO: 13
  REGION 1..249: note = Synthetic: UL141_11(2A)

SEQ ID NO: 15 - protein, 249 aa, synthetic construct
  REGION 1..249: note = Synthetic: UL141_1
SFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLRNFPHGLIMQRFVILTQLETLSRPDEPCCTPALGRYSLGDQIWSPTPWRLRNHDCGMYRGFQRNYFYIGRADAEDCWKPACPDEEPDRCWTVIQEYELPGDCYRSQPHPPKFLPVTPAPPADIDTGMSPWATR

SEQ ID NO: 16 - protein, 249 aa, synthetic construct
  REGION 1..249: note = Synthetic: UL141_13
SFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLRNFPHGLIMQRFVILTQLETLSRPDEPCCTPALGRYSLGDQIWSPTPWRLRNHDCGMYRGFQRNYFYIGRADAEDCWKPACPDEEPDRCWTVIQEPELPGDCYRSQPHPPKFLPVTPAPPADIDTGMSPWATR

SEQ ID NO: 17 - protein, 249 aa, synthetic construct
  REGION 1..249: note = Synthetic: UL141_14
SFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLHTYPDGLIMQRFVILTQLETLSRPDEPCCTPALGRYSLGDQIWSPTPWRLRNHDCGMYRGFQRNYFYIGRADAEDCWKPACPDEEPDRCWTVIQDYDLPGDCYRSQPHPPKFLPVTPAPPADIDTGMSPWATR

SEQ ID NO: 18 - protein, 249 aa, synthetic construct
  REGION 1..249: note = Synthetic: UL141_15
SFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLHTYPDGLIMQRFVILTQLETLSRPDEPCCTPALGRYSLGDQIWSPTPWRLRNHDCGMYRGFQRNYFYIGRADAEDCWKPACPDEEPDRCWTVIQDDYLPGDCYRSQPHPPKFLPVTPAPPADIDTGMSPWATR

SEQ ID NO: 19 - protein, 194 aa, synthetic construct; identical to SEQ ID NO: 11
  REGION 1..194: note = Synthetic: UL141_16

SEQ ID NO: 20 - protein, 194 aa, synthetic construct
  REGION 1..194: note = Synthetic: UL141_17
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTLSSGGGSPACPDEEPDRCWTVIQEPELPGDCYRSQPHPPKFLPVTPAPPADIDTGMSPWATR

SEQ ID NO: 21 - protein, 195 aa, synthetic construct
  REGION 1..195: note = Synthetic: UL141_18
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTLSSGGGSPACPDEEPDRCWTVIQDDYDLPGDCYRSQPHPPKFLPVTPAPPADIDTGMSPWATR

SEQ ID NO: 22 - protein, 195 aa, synthetic construct
  REGION 1..195: note = Synthetic: UL141_19
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTLSSGGGSPACPDEEPDRCWTVIQDDPDLPGDCYRSQPHPPKFLPVTPAPPADIDTGMSPWATR

SEQ ID NO: 23 - protein, 249 aa, synthetic construct
  REGION 1..249: note = Synthetic: UL141_20
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLETLSRPDEPCCTPALGRYSLGDQIWSPTPWRLRNHDCGMYRGFQRNYFYIGRADAEDCWKPACPDEEPDRCWTVIQDYDLPGDCYRSQPHPPKFLPVTPAPPADIDTGMSPWATR

SEQ ID NO: 24 - protein, 250 aa, synthetic construct
  REGION 1..250: note = Synthetic: UL141_21
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLETLSRPDEPCCTPALGRYSLGDQIWSPTPWRLRNHDCGMYRGFQRNYFYIGRADAEDCWKPACPDEEPDRCWTVDEGSDELPGDCYESQPHPPKFLPVTPAPPADIDTGMSPWATR

SEQ ID NO: 25 - protein, 249 aa, synthetic construct
  REGION 1..249: note = Synthetic: UL141_22
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLETLSRPDEPCCTPALGRYSLGDQIWSPTPWRLRNHDCGMYRGFQRNYFYIGRADAEDCWKPACPDEEPDRCWTVIESDELPGDCYESQPHPPKFLPVTPAPPADIDTGMSPWATR

SEQ ID NO: 26 - protein, 176 aa, synthetic construct
  REGION 1..176: note = Synthetic: UL141_23
SFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLRNFPHGLIMQRFVILTQLESRPDEPCCTPALGRYSLGDQIWSPTPWRLRNHDCGMYRGFS

SEQ ID NO: 27 - protein, 185 aa, synthetic construct
  REGION 1..185: note = Synthetic: UL141_24
SFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLRNFPHGLIMQRFVILTQLESRPDEPCCTPALGRYSLGDQIWSPTPWRLRNHDCGMYRGFQRNYFYIGRA

SEQ ID NO: 28 - protein, 199 aa, synthetic construct
  REGION 1..199: note = Synthetic: UL141_25
SFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLRNFPHGLIMQRFVILTQLETLSRPDEPCCTPALGRYSLGDQIWSPTPWRLRNHDCGMYRGFQRNYFYIGRADAEDCWKPACPS

SEQ ID NO: 29 - protein, 176 aa, synthetic construct; identical to SEQ ID NO: 26
  REGION 1..176: note = Synthetic: UL141_26:

SEQ ID NO: 30 - protein, 185 aa, synthetic construct; identical to SEQ ID NO: 27
  REGION 1..185: note = Synthetic: UL141_27

SEQ ID NO: 31 - protein, 199 aa, synthetic construct; identical to SEQ ID NO: 28
  REGION 1..199: note = Synthetic: UL141_28

SEQ ID NO: 32 - protein, 249 aa, synthetic construct; identical to SEQ ID NO: 13
  REGION 1..249: note = Synthetic: UL141_31 UL141_D11(2)_Lexa

SEQ ID NO: 33 - protein, 249 aa, synthetic construct; identical to SEQ ID NO: 13
  REGION 1..249: note = Synthetic: UL141_32 UL141_D11(2A)_Lexa

SEQ ID NO: 34 - protein, 371 aa, synthetic construct
  REGION 1..371: note = Synthetic: Fc fusion
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLESGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 35 - protein, 357 aa, synthetic construct
  REGION 1..357: note = Synthetic: Fc fusion
QLETLSRPDEPCCTPALGRYSLGDQIWSPTPWRLRNHDCGMYRGFQRNYFYIGRADAEDCWKPACPDEEPDRCWTVIQRYRLPGDCYRSQPHPPKFLPVTPAPPADIDTGMSPWATRSGGGSGGSEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLGK

SEQ ID NO: 36 - protein, 381 aa, synthetic construct
  REGION 1..381: note = Synthetic: Fc fusion
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLESRPDEPCCTPSGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 37 - protein, 391 aa, synthetic construct
  REGION 1..391: note = Synthetic: Fc fusion
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLESRPDEPCCTPALGRYSLGDQSGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 38 - protein, 391 aa, synthetic construct; identical to SEQ ID NO: 37
  REGION 1..391: note = Synthetic: Fc fusion

SEQ ID NO: 39 - protein, 401 aa, synthetic construct
  REGION 1..401: note = Synthetic: Fc fusion
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLESRPDEPCCTPALGRYSLGDQIWSPTPWRLRSGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 40 - protein, 411 aa, synthetic construct
  REGION 1..411: note = Synthetic: Fc fusion
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLESRPDEPCCTPALGRYSLGDQIWSPTPWRLRNHDCGMYRGFSGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 41 - protein, 421 aa, synthetic construct
  REGION 1..421: note = Synthetic: Fc fusion
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLESRPDEPCCTPALGRYSLGDQIWSPTPWRLRNHDCGMYRGFQRNYFYIGRASGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 42 - protein, 434 aa, synthetic construct
  REGION 1..434: note = Synthetic: Fc fusion
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLETLSRPDEPCCTPALGRYSLGDQIWSPTPWRLRNHDCGMYRGFQRNYFYIGRADAEDCWKPACPSGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 43 - protein, 430 aa, synthetic construct
  REGION 1..430: note = Synthetic: Fc fusion
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTLSSGGGSPACPDEEPDRCWTVIQEYELPGDCYRSQPHPPKFLPVTPAPPADIDTGMSPWATRSGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 44 - protein, 485 aa, synthetic construct
  REGION 1..485: note = Synthetic: Fc fusion
SFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLHTYPDGLIMQRFVILTQLETLSRPDEPCCTPALGRYSLGDQIWSPTPWRLRNHDCGMYRGFQRNYFYIGRADAEDCWKPACPDEEPDRCWTVIQEYELPGDCYRSQPHPPKFLPVTPAPPADIDTGMSPWATRSGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 45 - protein, 486 aa, synthetic construct
  REGION 1..486: note = Synthetic: Fc fusion
SFPFATADIAEKMWAENYETTSPAPVLVAEGEQVTIPCTVMTHSWPMVSIRARFCRSHDGSDELILNADLGHRLMNGLQYRLPFATWNFSQLHLGQIYSLTFNVSTDTAGMYECVLRNYPHGLIMQRFVILTQLETLSRPDEPCCTPALGRYSLGDQIWSPTPWRLRNHDCGMYRGFQRNYFYIGRADAEDCWKPACPDEEPDRCWTVIQEPELPGDCYRSQPHPPKFLPVTPAPPADIDTGMSPWATRSGAAAGAAAESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 46 - protein, 488 aa, synthetic construct
  REGION 1..488: note = Synthetic: Fc fusion
SFPFATADIAEKMWAENYETTSPAPVLVAEGEQVTIPCTVMTHSWPMVSIRARFCRSHDGSDELILNADLGHRLMNGLQYRLPFATWNFSQLHLGQIYSLTFNVSTDTAGMYECVLRNYPHGLIMQRFVILTQLETLSRPDEPCCTPALGRYSLGDQIWSPTPWRLRNHDCGMYRGFQRNYFYIGRADAEDCWKPACPDEEPDRCWTVIQEPELPGDCYRSQPHPPKFLPVTPAPPADIDTGMSPWATRSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 47 - protein, 485 aa, synthetic construct
  REGION 1..485: note = Synthetic: Fc fusion
SFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLRNFPHGLIMQRFVILTQLETLSRPDEPCCTPALGRYSLGDQIWSPTPWRLRNHDCGMYRGFQRNYFYIGRADAEDCWKPACPDEEPDRCWTVIQEYELPGDCYRSQPHPPKFLPVTPAPPADIDTGMSPWATRSGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 48 - protein, 485 aa, synthetic construct
  REGION 1..485: note = Synthetic: Fc fusion
SFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLRNFPHGLIMQRFVILTQLETLSRPDEPCCTPALGRYSLGDQIWSPTPWRLRNHDCGMYRGFQRNYFYIGRADAEDCWKPACPDEEPDRCWTVIQEPELPGDCYRSQPHPPKFLPVTPAPPADIDTGMSPWATRSGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 49 - protein, 485 aa, synthetic construct
  REGION 1..485: note = Synthetic: Fc fusion
SFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLHTYPDGLIMQRFVILTQLETLSRPDEPCCTPALGRYSLGDQIWSPTPWRLRNHDCGMYRGFQRNYFYIGRADAEDCWKPACPDEEPDRCWTVIQDYDLPGDCYRSQPHPPKFLPVTPAPPADIDTGMSPWATRSGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 50 - protein, 485 aa, synthetic construct
  REGION 1..485: note = Synthetic: Fc fusion
SFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLHTYPDGLIMQRFVILTQLETLSRPDEPCCTPALGRYSLGDQIWSPTPWRLRNHDCGMYRGFQRNYFYIGRADAEDCWKPACPDEEPDRCWTVIQDDYLPGDCYRSQPHPPKFLPVTPAPPADIDTGMSPWATRSGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 51 - protein, 430 aa, synthetic construct; identical to SEQ ID NO: 43
  REGION 1..430: note = Synthetic: Fc fusion

SEQ ID NO: 52 - protein, 430 aa, synthetic construct
  REGION 1..430: note = Synthetic: Fc fusion
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTLSSGGGSPACPDEEPDRCWTVIQEPELPGDCYRSQPHPPKFLPVTPAPPADIDTGMSPWATRSGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 53 - protein, 431 aa, synthetic construct
  REGION 1..431: note = Synthetic: Fc fusion
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTLSSGGGSPACPDEEPDRCWTVIQDDYDLPGDCYRSQPHPPKFLPVTPAPPADIDTGMSPWATRSGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 54 - protein, 431 aa, synthetic construct
  REGION 1..431: note = Synthetic: Fc fusion
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTLSSGGGSPACPDEEPDRCWTVIQDDPDLPGDCYRSQPHPPKFLPVTPAPPADIDTGMSPWATRSGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 55 - protein, 485 aa, synthetic construct
  REGION 1..485: note = Synthetic: Fc fusion
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLETLSRPDEPCCTPALGRYSLGDQIWSPTPWRLRNHDCGMYRGFQRNYFYIGRADAEDCWKPACPDEEPDRCWTVIQDYDLPGDCYRSQPHPPKFLPVTPAPPADIDTGMSPWATRSGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 56 - protein, 486 aa, synthetic construct
  REGION 1..486: note = Synthetic: Fc fusion
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLETLSRPDEPCCTPALGRYSLGDQIWSPTPWRLRNHDCGMYRGFQRNYFYIGRADAEDCWKPACPDEEPDRCWTVDEGSDELPGDCYESQPHPPKFLPVTPAPPADIDTGMSPWATRSGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 57 - protein, 485 aa, synthetic construct
  REGION 1..485: note = Synthetic: Fc fusion
SFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLETLSRPDEPCCTPALGRYSLGDQIWSPTPWRLRNHDCGMYRGFQRNYFYIGRADAEDCWKPACPDEEPDRCWTVIESDELPGDCYESQPHPPKFLPVTPAPPADIDTGMSPWATRSGGGSGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 58 - protein, 633 aa, synthetic construct
  REGION 1..633: note = Synthetic: Farletuzumab fusion
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLGKGGGSGGGSSFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLRNFPHGLIMQRFVILTQLESRPDEPCCTPALGRYSLGDQIWSPTPWRLRNHDCGMYRGFS

SEQ ID NO: 59 - protein, 642 aa, synthetic construct
  REGION 1..642: note = Synthetic: Farletuzumab fusion
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLGKGGGSGGGSSFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLRNFPHGLIMQRFVILTQLESRPDEPCCTPALGRYSLGDQIWSPTPWRLRNHDCGMYRGFQRNYFYIGRA

SEQ ID NO: 60 - protein, 656 aa, synthetic construct
  REGION 1..656: note = Synthetic: Farletuzumab fusion
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLGKGGGSGGGSSFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLRNFPHGLIMQRFVILTQLETLSRPDEPCCTPALGRYSLGDQIWSPTPWRLRNHDCGMYRGFQRNYFYIGRADAEDCWKPACPS

SEQ ID NO: 61 - protein, 633 aa, synthetic construct
  REGION 1..633: note = Synthetic: Farletuzumab fusion
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLGKGAAAGAAASFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLRNFPHGLIMQRFVILTQLESRPDEPCCTPALGRYSLGDQIWSPTPWRLRNHDCGMYRGFS

SEQ ID NO: 62 - protein, 642 aa, synthetic construct
  REGION 1..642: note = Synthetic: Farletuzumab fusion
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLGKGAAAGAAASFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLRNFPHGLIMQRFVILTQLESRPDEPCCTPALGRYSLGDQIWSPTPWRLRNHDCGMYRGFQRNYFYIGRA

SEQ ID NO: 63 - protein, 656 aa, synthetic construct
  REGION 1..656: note = Synthetic: Farletuzumab fusion
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLGKGAAAGAAASFPFATADIAEKMWAENYETTSPAPVLVAEGEQVTIPCTVMTHSWPMVSIRARFCRSHDGSDEAILNADLGHRLMNGLQYRLPYATWNFSQLHLGQIFSLTFNVSTDTAGMYECVLRNFPHGLIMQRFVILTQLETLSRPDEPCCTPALGRYSLGDQIWSPTPWRLRNHDCGMYRGFQRNYFYIGRADAEDCWKPACPS

SEQ ID NO: 64 - protein, 741 aa, synthetic construct
  REGION 1..741: note = Synthetic: Lexatumumab fusion
SFPFATADIAEKMWAENYETTSPAPVLVAEGEQVTIPCTVMTHSWPMVSIRARFCRSHDGSDELILNADLGHRLMNGLQYRLPFATWNFSQLHLGQIYSLTFNVSTDTAGMYECVLRNYPHGLIMQRFVILTQLETLSRPDEPCCTPALGRYSLGDQIWSPTPWRLRNHDCGMYRGFQRNYFYIGRADAEDCWKPACPDEEPDRCWTVIQEPELPGDCYRSQPHPPKFLPVTPAPPADIDTGMSPWATRSGAAAGAAAESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGSGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGGGGSGGGDSGGGGSGGGGSEVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSS

SEQ ID NO: 65 - protein, 742 aa, synthetic construct
  REGION 1..742: note = Synthetic: Lexatumumab fusion
SFPFATADIAEKMWAENYETTSPAPVLVAEGEQVTIPCTVMTHSWPMVSIRARFCRSHDGSDELILNADLGHRLMNGLQYRLPFATWNFSQLHLGQIYSLTFNVSTDTAGMYECVLRNYPHGLIMQRFVILTQLETLSRPDEPCCTPALGRYSLGDQIWSPTPWRLRNHDCGMYRGFQRNYFYIGRADAEDCWKPACPDEEPDRCWTVIQEPELPGDCYRSQPHPPKFLPVTPAPPADIDTGMSPWATRSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGSGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGGGGSGGGDSGGGGSGGGGSEVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSS

SEQ ID NO: 66 - protein, 338 aa, Homo sapiens
  REGION 1..338: note = misc_feature - UL141 sequence
MCRRESLRTLPWLFWVLLSCPRLLEYSSSSFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLETLSRPDEPCCTPALGRYSLGDQIWSPTPWRLRNHDCGMYRGFQRNYFYIGRADAEDCWKPACPDEEPDRCWTVIQRYRLPGDCYRSQPHPPKFLPVTPAPPADIDTGMSPWATRGIAAFLGFWSIFTVCFLCYLCYLQCCGRWCPTPGRGRRGGEGYRCLPTYDSYPGVKKMKR